(19) United States
(12) Patent Application Publication
(10) Pub. No.: US 2013/0095542 A1
(43) Pub. Date: Apr. 18, 2013

(54) ENGINEERED MICROORGANISMS AND INTEGRATED PROCESS FOR PRODUCING N-PROPANOL, PROPYLENE AND POLYPROPYLENE

(71) Applicants: Gonçalo Amarante Guimarães Pereira, Campinas (BR); Johana Rincones Perez, Campinas (BR); Marcelo Falsarella Carazzolle, Valinhos (BR); Ane Fernanda Beraldi Zeidler, Campinas (BR); Lucas Pedersen Parizzi, Limeira (BR); Luige Armando Llerena Calderón, Campinas (BR); Maria Carolina de Barros Grassi, Campinas (BR); Inês Lunardi, Campinas (BR); Luciana Gonzaga de Oliveira, Campinas (BR); José Augusto Rosário Rodrigues, Campinas (BR); Paulo José Samenho Moran, Campinas (BR); Antonio Luiz Ribeiro de Castro Morschbacker, Campinas (BR); Luiza Roza, Porto Alegre (BR); Márcio Henrìque dos Santos Andrade, Maceio (BR)

(72) Inventors: Gonçalo Amarante Guimarães Pereira, Campinas (BR); Johana Rincones Perez, Campinas (BR); Marcelo Falsarella Carazzolle, Valinhos (BR); Ane Fernanda Beraldi Zeidler, Campinas (BR); Lucas Pedersen Parizzi, Limeira (BR); Luige Armando Llerena Calderón, Campinas (BR); Maria Carolina de Barros Grassi, Campinas (BR); Inês Lunardi, Campinas (BR); Luciana Gonzaga de Oliveira, Campinas (BR); José Augusto Rosário Rodrigues, Campinas (BR); Paulo José Samenho Moran, Campinas (BR); Antonio Luiz Ribeiro de Castro Morschbacker, Campinas (BR); Luiza Roza, Porto Alegre (BR); Márcio Henrìque dos Santos Andrade, Maceio (BR)

(21) Appl. No.: 13/394,721

(22) PCT Filed: Sep. 9, 2010
(86) PCT No.: PCT/BR2010/000289

§ 371 Date: Aug. 29, 2012

Related U.S. Application Data
(60) Provisional application No. 61/240,811, filed on Sep. 9, 2009.

(51) Int. Cl. C12P 7/04

(52) U.S. Cl. CPC C12P 7/04
USPC 435/157; 435/252.3; 435/167

(57) ABSTRACT

The invention provides fermentative methods for producing n-propanol. The methods of the invention involve providing a suitable carbon source, a microorganism expressing the dicarboxylic acid pathway, reducing equivalents, and at least one gene coding for an enzyme that catalyzes the conversion of propionate/propionyl-CoA into n-propanol. The methods further involve contacting the carbon source and reducing equivalents with the microorganism under conditions favorable for the production of n-propanol. Also provided are methods for producing propylene and polypropylene from the n-propanol and microorganisms suitable for use in the methods of the invention.

### SEQUENCE LISTING

The instant application contains a Sequence Listing which has been submitted in ASCII format via EFS-Web and is hereby incorporated by reference in its entirety. Said ASCII copy, created on Aug. 28, 2012, is named F522100428.txt

### FIELD OF THE INVENTION

The present invention relates to a process of bioconverting a biobased substrate (such as sugarcane juice, hydrolyzed starch, hydrolyzed cellulose or glycerol) into n-propanol using genetically modified microorganisms combined with a process for supplying reducing equivalents in the form of NAD(P)H during fermentation. The biobased n-propanol thus obtained could be dehydrated to propylene and polymerized to polypropylene to yield a bioplastic.

### BACKGROUND OF THE INVENTION

n-Propanol (1-propanol, primary propyl alcohol, propan-1-ol) is a non-hazardous solvent that is freely miscible with water and other common solvents, with numerous applications in industry, such as printing inks, coatings, cleaners, adhesives, herbicides, insecticides, pharmaceuticals, de-icing fluids and as a chemical intermediate for the production of esters, propylamines, halides and thermoplastic resins. The use of n-propanol in fuel blends has also been suggested (U.S. Pat. No. 6,129,773), as this alcohol has the same capacity of ethanol to be used to increase as an antiknock additive and increase the octane number of gasoline according to Barannik V. P. et al. 2005, Chemistry and Technology of Fuels and Oils 41(6): 452-455.

n-Propanol is one of the main constituents of “fusel oils” or “potato oils”, which are the higher-order alcohols by-products of ethanol fermentation by the yeast Saccharomyces cerevisiae (Hazelwood et al. 2008. The Ehrlich Pathway for Fusel Alcohol Production: a Century of Research on Saccharomyces cerevisiae Metabolism. Applied and Environmental Microbiology 74(8): 2259-2266). In the past, n-Propanol was obtained by fractional distillation of fusel oil, but nowadays it is manufactured from fossil feedstocks in a two-stage process known as Oxo Process, comprising ethylene hydroformylation at 80-120° C. and 2.0 MPa in the presence of cobalt or rhodium carbonyl followed by hydrogenation of the resulting propionaldehyde on a copper-chromium, nickel-chromium or porous cobalt catalyst (U.S. Pat. No. 4,263,449 and U.S. Pat. No. 5,866,725).

Worldwide interest in organic compounds produced from renewable feedstocks has increased considerably in recent years, especially for compounds that can be used as fuels or as bulk chemicals for the petrochemical industry. The latter are particularly interesting, since these compounds could be fixed in highly durable materials that can be recycled, thus effectively mitigating atmospheric CO₂ (Rincones et al. 2009. The golden bridge for nature: the new biology applied to bioplastics. Polymer Reviews 49: 85-106). Thus, the use of the chemical products obtained from renewable feedstocks is becoming increasingly accepted and widespread as a viable alternative aiming at decreasing our society's dependence on fossil carbon sources. Products obtained from green sources can be certified as to their renewable carbon content according to the methodology described by the technical norm ASTM D 6866-06: “Standard Test Methods for Determining the Biobased Content of Natural Range Materials Using Radiocarbon and Isotope Ratio Mass Spectrometry Analysis”.

The production of short-chain organic solvents (mainly reduced alcohols) through microorganism fermentation has been extensively studied. The most dramatic example is the production of ethanol as a commodity chemical, which is a major industrial process reaching nearly 90 million m³/year and occurring by the fermentation of renewable carbon sources (mainly cornstarch and sugarcane juice) by the yeast Saccharomyces cerevisiae. This process is extremely efficient and has been refined to the point where ethanol distilled from the fermentation broth is obtained at 90-95% of the theoretical yield. The ethanol thus produced is used as an industrial solvent, as the main additive for gasoline in fuel blends and, in Brazil, is used as the sole fuel for small vehicles. Another use of a biobased ethanol is the manufacture of bio-ethylene to be used as a monomer in the polyethylene manufacture, through a dehydration reaction as described by Morschbacker A. L. 2009, Bio-Ethanol Based Ethylene, Journal of Macromolecular Science, Part C: Polymer Reviews, 49:79-84.

Other well-known examples of solvent production by fermentation are the Acetone-Butanol-Ethanol (ABE) and the Isopropanol-Butanol-Ethanol (IBE) fermentations performed by some bacterial species of the genus Clostridium, yielding more than 35% by weight of the solvent mixture (U.S. Pat. No. 5,192,673). In addition, fermentation of 2,3-butanediol from carbohydrates by enteric bacteria of the genera Klebsiella and Enterobacter yields up to 47% by weight (Ji et al., 2009, Bioresource Technology 100:3410-3414). A recent success is the fermentative production of 1,3 propanediol from glucose in a single microorganism with high yield (35% w/w) and titer (129 g/L) (U.S. Pat. No. 7,169,588 B2; U.S. Pat. No. 7,067,300 B2; U.S. Pat. No. 5,686,276). The establishment of an industrial process for the production of this low cost biobased 1,3 propanediol from cornstarch and its subsequent use in the production of the polyester fiber polypropylene terephthalate constitutes one of the most significant advances to date in the production of biopolymers.

n-Propanol and isopropanol are interesting biobased intermediates for the production of propylene by dehydration and its subsequent polymerization into polypropylene. Up to date, the best yield for isopropanol has been obtained through a genetically engineered strain of E. coli containing genes coding for the enzymes of the acetone production pathway of Clostridium acetobutylicum plus the secondary alcohol dehydrogenase of the isopropanol production pathway of Clostridium beijerinckii, yielding 14% by weight of isopropanol from glucose (Int. Publ. No. WO 2008/131286 A1). This yield corresponds to approximately 50% of the theoretical maximum, since the proposed pathway for the production of isopropanol comprises the following conversions: a) cleavage of glucose into two molecules of pyruvate through glycolysis; b) oxidative decarboxylation of the molecules of pyruvate into acetyl-CoA; c) condensation of the two molecules of acetyl-CoA into acetoacetyl-CoA and CoA; d) conversion of acetoacetyl-CoA and acetate into acetoacetate and acetyl-CoA; e) decarboxylation of acetoacetate into acetone; and f) reduction of acetone into isopropanol. As can be seen from the conversions above, involving three decarboxylation steps of intermediate metabolites, the maximum theoretical yield of isopropanol through this pathway is 1 mol of isopropanol from each mol of glucose (0.33 g/g).

In nature, microorganisms produce n-propanol in low amounts and as by-product of the main fermentation products. In the yeast Saccharomyces cerevisiae, n-propanol is produced as the degradation product of the amino acid 2-ketobutyrate through the Ehrlich pathway (Hazelwood et al., 2008, Appl. Env. Microbiol. 74:2259-2266). This pathway has been optimized in genetically engineered strains of the model microorganism Escherichia coli for the production of n-butanol and n-propanol from glucose, but with extremely low yields (4% by weight) (Shen & Liao, 2008, Met. Eng. 10:312-320). The production of iso-propanol or n-propanol via the degradation of the amino acid 2-ketobutyrate, from glucose through this pathway using genetically engineered microorganisms is also disclosed in a recent document, but similarly indicating very low yields (Intl. Pub. No. WO 2009/103026 A1). In bacterial species of the genus Propionibacterium, n-propanol has been observed as the by-product of propionic acid fermentation from glycerol, which is a more reduced substrate when compared to glucose or sucrose, but with low yields (4% by weight); no n-propanol is obtained when glucose, sucrose or lactate are used as substrates in the fermentation using P. acidipropionici American Type Culture Collection (ATCC) No. 25562 (Barbirato et al., 1997, Appl. Microbiol. Biotechnol. 47: 441-446). Thus, the prior art fails to show fermentation processes for the production of n-propanol with high yields by fermentation of carbohydrates.

Propionic acid fermentation by several bacterial species, such as Selenomonas ruminantium, Propionigenium spp. and Propionibacterium spp. has been extensively studied. Propionic acid bacteria of the genus Propionibacterium have been the most studied due to their use in the production of cheese. These bacteria produce propionic acid as the main fermentation product from glucose and other substrates such as lactose, glycerol, and sucrose with high yields of propionic acid (65% w/w from glucose and 67% w/w from glycerol) (Suwannakham & Yang., 2005, Biotech. Bioeng 91:325-337; Barbirato et al., 1997, Appl. Microbiol. Biotechnol. 47: 441-446). The pathway for the production of propionic acid in Propionibacterium spp. is known as the dicarboxylic acid cycle, which begins by the trascarboxylation of pyruvate from methyl-malonyl-CoA to yield oxaloacetate followed by the subsequent transformations into malate, fumarate, succinate, succinyl-CoA and methyl-malonyl-CoA, which will be transcarboxylated to pyruvate to yield propionyl-CoA and oxaloacetate, thus closing the cycle (Boyaval and Cone, 1995, Lait 75:453-461). Therefore, no decarboxylation reactions are involved in this pathway, which would have a maximum theoretical yield of 2 mol of propionic acid for each mol of glucose (0.82 g/g). Nevertheless, the co-products acetic acid and succinic acid are usually formed in varying proportions depending on the substrate and growth conditions.

Several studies and patent applications are directed to method for increasing the yield of propionic acid, especially with regards to increase its yield in relation to co-products, such as acetic acid, and to improve the growth conditions and separation strategies (“Engineering Propionibacteriuml acidipropionici for Enhanced Propionic Acid Tolerance and Fermentation”, Zhang and Yang, 2009, Biotechnology and bioengineering, in press” and “Construction and Characterization of ack Knock-Out Mutants of Propionibacterium acidipropionici for Enhanced Propionic Acid Fermentation”, Suwannakham et al, 2006, Biotechnology and Bioengineering, Vol. 94, No. 2, June 5). However, no studies exist aiming at improving the formation of n-propanol using the propionic acid pathway as a metabolic intermediate.

No natural microorganisms are able to produce iso- or n-propanol with high yields from glucose and other sugars; in consequence, the correct combination of enzymes that would allow such bioconversion does not exist in nature. However, Holt et al. (1984, Appl. Env. Microbiol. 48:1166-1170) have shown that the external supply of propionic acid to a growing culture of Clostridium acetobutylicum at acidic pH (5.0) yields n-propanol (50% w/w), suggesting that the alcohol/aldehyde dehydrogenase (ADH) enzymes of this bacterium are able to transform not only the acyl-CoA it produces (butyrate and acetate) into the corresponding alcohols, but also propionate into n-propanol. However the experiments of this publication were conducted at a very low concentration and high levels of undesired by-products such as acetate, butyrate, ethanol, butanol and acetone were obtained, thus indicating that there is still a problem to be solved in order to obtain propanol with high yields.

In addition, the metabolic pathways that lead to the production of industrially important compounds involve oxidation-reduction (redox) reactions. During fermentation, glucose is oxidized in a series of enzymatic reactions into smaller molecules with the concomitant release of energy. Since these reactions do not occur simultaneously, the electrons released are transferred from one reaction to another through universal electron carriers, such as Nicotinamide Adenine Dinucleotide (NAD) and Nicotinamide Adenine Dinucleotide Phosphate (NADP), which act as cofactors for oxidoreductase enzymes. In microbial catabolism, glucose is oxidized by enzymes using the oxidized form NAD(P)+ as cofactor and generating reducing equivalents in the form of the reduced form NAD(P)H. In order for fermentation to continue, the NAD(P)+ must be regenerated by the reduction of metabolic intermediates consuming NAD(P)H. Thus, it is very important for the microbial cell to maintain a balanced NAD(P)+/NAD(P)H ratio.

In general, reducing equivalents in the form of NAD(P)H are obtained in oxidative decarboxylation reactions, while NAD(P)+ is regenerated by the reduction of intermediates, such as the reduction of acetic acid into ethanol. As a consequence of the redox balance required for the catabolism of glucose into n-propanol, which has a lower oxidation state, this compound would be accompanied by the co-production of 2- and, possibly, 4-carbon compounds. This fact suggests that low yields should be observed for the production of n-propanol, even when genetically engineered microorganisms are to be used due to the requirement of more reducing equivalents in the form of NAD(P)H than can be formed from the oxidation of glucose. Thus, this situation for n-propanol contrasts with the fermentative production of isopropanol from glucose disclosed in Int. Publ. No. WO 2008/131286 A1, in which the product results by a series of conversions involving three oxidative decarboxylation reactions from glucose, which generate enough reducing equivalents for the reduction of acetone into isopropanol, but at the expense of mass released as CO₂.

Previous studies have reported the use of electrical stimulation inside bioreactors in order to drive the redox balance to obtain different end-products. The application of an electrical current in Clostridium acetobutylicum, Clostridium thermocellum and Saccharomyces cerevisiae has been reported, resulting in a significant increase in ethanol production (Pequin et. al. 1994, Biotechnology letters 16(3): 269-274; Shin et al 2002, Appl. Microbial. Biotechnol. 58: 476-481). Also, there are works reporting the change in the end-products of fermentation by Propionibacterium spp. using electrical stimulation and mediators. Emde and Schink (D.E. Pat. No. 4,024,937-C1) enhanced propionate formation during glucose fermentation of Propionibacteriuml freudenreichi using a three-electrode system and cobalt sepulchrate as mediator. Results showed that this process increases propionate molar yield over acetate from 73 to 97%, respectively. In a similar work, Schuppert et al. (Appl. Microbiol. Biotechnol, 1992, 37:549-553) used thye three-electrode system and cobalt sepulchrate to shift the end-product ratio of P. acidipropionici. In this case, propionate was produced exclusively, thus increasing final yields and facilitating the downstream process. Finally, in a recent work, the end-product product profile of glucose fermentation by P. freudenreichi was modified by electrical stimulation without adding exogenous artificial mediators (Wang et. al. 2008, Biotechnol. Bioeng 101: 579-586). In this work, the authors reported that the molecule 1,4-dihydroxy-2-naphthoic acid produced and secreted by P. freudenreichi acts as the mediator and no improvement of the reaction was observed when other mediators were added. Overall, these results show that the metabolism and end-product profile of glucose fermentation by Propionibacterium spp. can be manipulated through the use of bioelectrical reactors. However, little n-propanol was detected in the assays, even when reducing equivalents in the form of NAD(P)H were externally supplied, thus suggesting that aldehyde/alcohol dehydrogenases (ADHs) from propionibacteria are not efficient in the reduction of propionate/propionyl-CoA into n-propanol.

The biobased n-propanol thus produced could be further used for the production of a bioplastic through its dehydration to propylene and its polymerization to polypropylene in a cost-effective manner.

Propylene is a chemical compound that is widely used to synthesize a wide range of petrochemical products. For instance, this olefin is the raw material used for the production of polypropylene, their copolymers and other chemicals such as acrylonitrile, acrylic acid, epichloridrine and acetone. Propylene demand is growing faster than ethylene demand, mainly due to the growth of market demand for polypropylene. Propylene is polymerized to produce thermoplastics resins for innumerous applications such as rigid or flexible packaging materials, blow molding and injection molding.

Global interest for renewable material has been growing intensively in the last years especially in plastics production. Some available biopolymers are poly-(lactic acid) and poly-hydroxybutyrate which can be obtained from sugar sources. Another recent alternative is “green” polyethylene which is produced from sugarcane ethanol. These products generate no fossil carbon when incinerated.

Propylene is obtained mainly as a by-product of catalytical or thermal oil cracking, or as a co-product of ethylene production from natural gas. (Propylene, Jamie G. Lacson, CEH Marketing Research Report-2004, Chemical Economics Handbook-SRI International). The use of alternative routes for the production of propylene has been continuously evaluated using a wide range of renewable raw materials (“Green Propylene”, Nexant, January 2009). These routes include propylene production by dimerization of ethylene to yield butylene followed by metathesis with additional ethylene to produce propylene. Another route is biobutanol production by sugar fermentation followed by dehydration and methatesis with ethylene. Some thermal routes are also being evaluated such as gasification of biomass to produce a syngas followed by synthesis of methanol, which will then produce green propylene via methanol-to-olefin technology.

Propylene production by iso-propanol dehydration has been well-described in document EP00498573B1, wherein all examples show propylene selectivity higher than 90% with high conversions. Dehydration of n-propanol has also been studied in the following articles: “Mechanism and Kinetics of the Acid-Catalyzed Dehydration of 1- and iso-propanol in Hot Compressed Liquid Water” (Antal, M et al., Ind. Eng. Chem. Res. 1998, 37, 3820-3829) and “Fischer-Tropsch Aqueous Phase Refining by Catalytic Alcohol Dehydration” (Nel, R. et al., Ind. Eng. Chem. Res. 2007, 46, 3558-3565). The reported yield is higher than 90%.

### BRIEF SUMMARY OF THE INVENTION

In spite of the innumerous developments achieved to date, there are still no teachings in the prior art that provide any description relative to the production of n-propanol with high yields through propionic acid metabolic pathway using genetically modified microorganisms combined with a process for supplying reducing equivalents in the form of NAD(P)H during fermentation of renewable carbon sources. The biobased n-propanol thus obtained could be dehydrated to propylene and polymerized to yield biobased polypropylenes. This thus produced bio-polypropylene, contrary to the majority of known biopolymers, have a low production cost and evidence clearly adequate properties for an immense variety of applications.

The present invention provides an improved process for the bioconversion of a carbon source to n-propanol, and eventually additionally to iso-propanol and/or ethanol, with high yield by engineered microorganisms, having genes coding for the enzymes of the dicarboxylic acid pathway of propionate formation and at least one gene coding for an enzyme that catalyzes the conversion propionate/propionyl-CoA into n-propanol in the presence of externally supplied reducing equivalents in the form of NAD(P)H, either through the use of electrodes and a mediator molecule, or through the use of an overpressure of H₂, or through the use of a pathway, native or engineered, expressing a NAD⁺-dependent formate dehydrogenase and the addition of formate to the culture medium.

The present invention provides methods for the biological production of n-propanol with high yields by microorganisms from an inexpensive carbon substrate such as glucose, sucrose, other sugars, glycerol, waste materials or a mixed of carbon sources, using the whole cell as catalyst and establishing an integrated process that may be upscaled to industry in a cost-effective manner. To this end, the present invention further provides engineered microorganisms capable of producing propionate/propionyl-CoA with high yields through the dicarboxylic acid cycle and that express the polypeptides corresponding to alcohol/aldehyde dehydrogenase enzymes capable of reducing propionate/propionyl-CoA into n-propanol.

The present invention provides a high yielding process for the fermentative production of n-propanol. In one embodiment of the invention, the processes or methods involve a balanced energy reaction in the conversion of glucose or other carbohydrates into n-propanol.

The present invention also comprises the product of the above process.

In certain embodiments, microorganisms that contain a native dicarboxylic acid cycle can be engineered to catalyze the further conversion into n-propanol by the addition of at least one heterologous gene coding for an aldehyde/alcohol dehydrogenase enzymes.

In certain embodiments, a suitable host with a native pathway for the conversion of propionyl-CoA/propionate into n-propanol is engineered for expression of the dicarboxylic acid cycle, where the expression of at least one enzyme is heterologous or has its expression pattern modified.

In certain embodiments, a suitable host, for which genetic manipulation techniques are well-established, is engineered for expression of the dicarboxylic acid cycle and the enzymes required for the reduction of propionate/propionyl-CoA into n-propanol, where the expression of at least one enzyme is heterologous or has its expression pattern altered.

In certain embodiments, microorganisms that contain a native or a modified dicarboxylic acid cycle and that contains a native or a modified pathway for the conversion of propionyl-CoA/propionate into n-propanol can be further engineered to express the enzymes that catalyze the conversion of acetyl-CoA into isopropanol. This isopropanol would be used together with n-propanol for propylene synthesis by dehydration.

In certain embodiments, microorganisms that contain a native or a modified dicarboxylic acid cycle, a native or a modified pathway for the conversion of propionyl-CoA/propionate into n-propanol and a native or modified pathway for the conversion of acetyl-CoA into isopropanol may be engineered to present an altered expression (over or underexpression) of a defective enzyme involved in the acetic acid synthesis from acetyl-CoA, which would increase isopropanol synthesis. This isopropanol would be used together with n-propanol for propylene synthesis by dehydration.

The preferred method of externally supplying electrons is through the use of electrodes and a mediator molecule, which can be naturally produced by the microorganism or externally supplied in the culture medium.

In certain embodiments a fermentation media containing sugarcane juice as carbon source is preferentially used and a nitrogen source consisting of either yeast extract or N₂ is preferentially used. However, other combinations may be used and those skilled in the art recognize that these combinations are also considered within the scope of this invention.

In certain embodiments the culture media is supplied with pantothenic acid with the object of increasing yield and productivity. This pantothenic acid may be added in pure form or as a crude extract.

In certain embodiments, the n-propanol thus produced will be further dehydrated into propylene and polymerized to polypropylene to yield a bioplastic.

### BRIEF DESCRIPTION OF THE FIGURES

Having thus described the invention in general terms, reference will now be made to the accompanying drawings, which are not necessarily drawn to scale, and wherein:

FIG. 1. The production of propionic acid from glucose by several species of bacteria, such as Propionigenium spp., Propionispira arboris, Propionibacterium spp. and Selenomonas ruminantium, can be accomplished by the following series of steps. This series is representative of a number of pathways known to those skilled in the art. Glucose is converted in a series of steps by enzymes of glycolytic pathway to pyruvate. The pyruvate may be converted to Acetyl-Coa and then to acetate or to propionic acid through the dicarboxylic acid cycle. It has been reported that some species of the genus Propionibacterium may produce n-propanol when a reduced substrate such as glycerol is used; however, the pathway for the production of n-propanol has not been described. The possible pathways and co-factors for the production of n-propanol are highlighted in gray.

FIG. 2. The production of alcohols by species of Clostridium may be described by the following steps. Glucose is converted in a series of steps by enzymes of glycolytic pathway to pyruvate. From pyruvate may be formed lactate or acetyl-CoA which is the precursor of acetate and ethanol. In addition, acetyl-CoA can be converted to acetoacetyl-CoA and then to acetone, which is finally reduced to isopropanol. Another possibility is the conversion of acetoacetyl-CoA in butyryl-Coa through a series of steps known by those skilled in the art. The butyryl-CoA may be converted to either butanol or butyrate.

FIG. 3. Schematic representation of a stirred-tank bioelectrical reactor with a three-electrode system.

FIG. 4. Schematic representation of the integrated processes wherein an engineered microorganism is used to produce n-propanol in the presence of reducing equivalents externally supplied through the use of a bioelectrical reactor. The resulting n-propanol is distilled and dehydrated in a catalytic reactor in order to produce polymer grade propylene, which is then subjected to a polymerization step to produce polypropylene.

FIG. 5. Schematic representation of expression vector pBK1T1 containing a synthetic construct designed to express an aldehyde alcohol dehydrogenase from Clostridium carboxidivorans in Propionibacterium acidipropionici. This bifunctional enzyme catalyzes the conversion of propionyl-CoA into n-propanol.

FIG. 6. Schematic representation of expression vector pBK1T2 containing a synthetic construct designed to express an aldehyde alcohol dehydrogenase from Clostridium acetobutylicum in Propionibacterium acidipropionici. This bifunctional enzyme catalyzes the conversion of propionyl-CoA into n-propanol.

FIG. 7. Thiostrepton resistance positive selection marker cassette for Propionibacterium acidipropionici, synthetic construct (SEQ ID NO.: 151). NcoI site (underlined), controlling regions (bold) and initiation and stop codons of the resistance gene ORF (in parenthesis) are highlighted.

FIG. 8. Expression cassette for heterologous bifunctional aldehyde/alcohol dehydrogenase of Clostridium carboxidivorans in Propionibacterium acidipropionici, synthetic construct (SEQ ID NO.: 152). XbaI and HindIII sites (underlined), controlling regions (bold) and initiation and stop codons of the gene ORF (in parenthesis) are highlighted.

FIG. 9. Expression cassette for heterologous bifunctional aldehyde/alcohol dehydrogenase of Clostridium acetobutylicum in Propionibacterium acidipropionici, synthetic construct (SEQ ID NO.: 153). XbaI and HindIII sites (underlined), controlling regions (bold) and initiation and stop codons of the gene ORF (in parenthesis) are highlighted.

FIG. 10. Expression plasmid pBK1T1, synthetic construct (SEQ ID NO.: 154). A schematic view of the plasmid vector is presented in FIG. 5.

FIG. 11. Expression plasmid pBK1T2 (SEQ ID NO.: 155), synthetic construct. A schematic view of the plasmid vector is presented in FIG. 6.

FIG. 12. HPLC spectra obtained after 36 hrs of (a) control fermentation and (b) fermentation supplemented with 1.0 mM cobalt sepulchrate as a mediator molecule. Chromatogram (a): Sucrose (11.437 min); succinic acid (17.782 min); acetic acid (22.610 min); propionic acid (26.515 min); Chromatogram (b): Sucrose (11.420 min); succinic acid (17.714 min); acetic acid (22.586 min); propionic acid (26.493 min); n-propanol (39.199). The undefined peaks are corresponding to compounds from yeast extract.

FIG. 13. GC-MS chromatogram corresponding to fermentation using 1.0 mM cobalt sepulchrate. The intensity of the peaks are not corresponding to the real concentration of the products in the fermentation medium.

FIG. 14. Time course for cell growth of a control fermentation and a fermentation supplemented with 1.0 mM cobalt sepulchrate as a mediator molecule

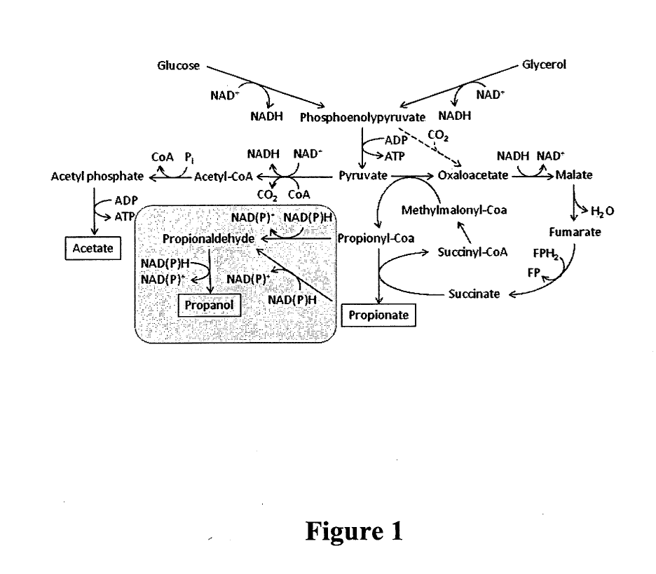

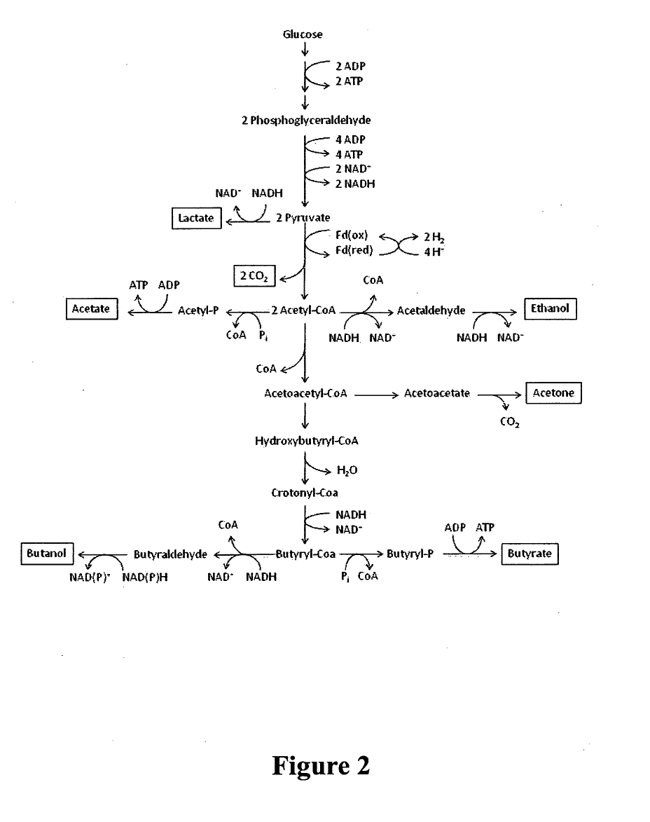

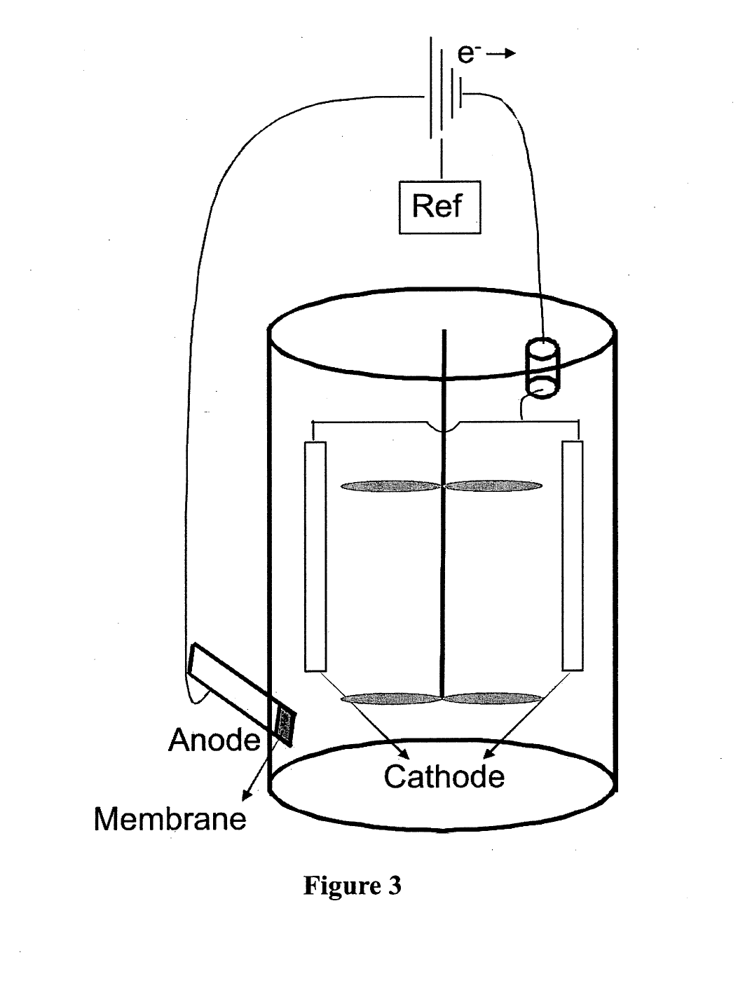

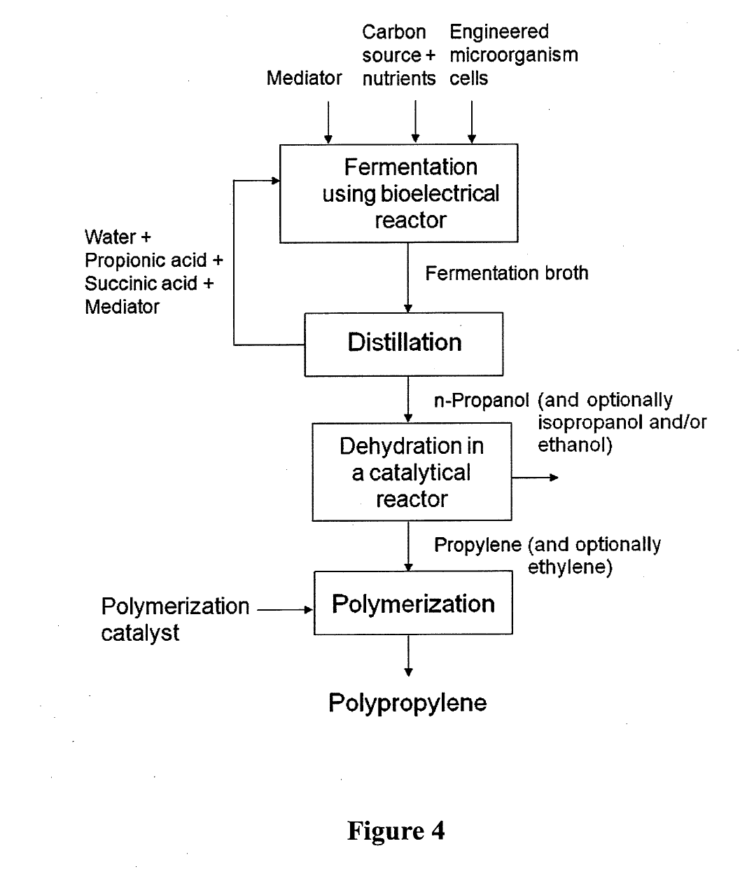

### DETAILED DESCRIPTION OF THE INVENTION

The present invention provides a novel integrated approach that takes advantage of the high propionic acid fermentation yields from renewable feedstocks through the dicarboxylic acid cycle, the aldehyde/alcohol dehydrogenase genes of alcohol-producing microbial species, such as clostridia, yeasts and enteric bacteria, and the external supply of reducing equivalents in the form of NAD(P)H in order to produce n-propanol from fermentation with high yield. Therefore, the present invention provides a novel and inventive integrated process using microorganisms combined with the use of externally supplied reducing equivalents for the production of n-propanol with high yield, and as an option, a complementary production of iso-propanol and/or ethanol with the aim to maximize the carbon yield in molecules of interest.

A process is disclosed herein for the bioconversion of a carbon source to n-propanol with high yield in engineered microorganisms expressing genes coding for the enzymes of the dicarboxylic acid pathway of propionate formation and at least one gene coding for an enzyme that catalyzes the conversion propionate/propionyl-CoA into n-propanol in the presence of externally supplied reducing equivalents in the form of NAD(P)H, either through the use of electrodes and a mediator molecule, or through the use of an overpressure of H₂, or through the use of a pathway, native or engineered, expressing a NAD⁺-dependent formate dehydrogenase and the addition of formate to the culture medium.

The term “microorganism” as used herein includes prokaryotic and eukaryotic species from the domains Archaea, Bacteria and Eukarya, the latter limited to filamentous fungi, yeasts, algae, protozoa or higher Protista. “Cell”, “microbial cell” or “microbe” are used interchangeably with microorganism. The term “organism” as used herein refers to any self-replicating entity.

The term “carbon source” generally refers to a substrate or compound suitable for sustaining microorganism growth. Carbon sources may be in various forms, including, but not limited to polymers, carbohydrates, alcohols, acids, aldehydes, ketones, amino acids, peptides, etc. For example, these may include monosaccharides (such as glucose, fructose, and xylose), oligosaccharides (i.e. sucrose, lactose), polysaccharides (i.e. starch, cellulose, hemicellulose), lignocellulosic materials, fatty acids, succinate, lactate, acetate, glycerol, etc. or a mixture thereof. The carbon source may be a product of photosynthesis, such as glucose or cellulose. Monosaccharides used as carbon sources may be the product of hydrolysis of polysaccharides, such as acid or enzymatic hydrolysates of cellulose, starch and pectin. The term “energy source” may be used here interchangeably with carbon source since in chemoorganotrophic metabolism the carbon source is used both as an electron donor during catabolism and as a carbon source during cell growth.

The term “nucleic acid” refers to an organic polymer composed by more than two monomers of nucleotides of nucleosides, including, but not limited to, single-stranded or double-stranded, sense or anti-sense, deoxyribonucleic acid (DNA) of any length, and, where appropriate, single-stranded or double-stranded, sense or anti-sense, ribonucleic acid (RNA) of any length. The term “nucleotide” refers to any or several compounds that consist of a ribose or deoxyribose sugar joined to a purine or pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. A nucleic acid containing from three to 200 nucleotides may also called “oligonucleotide”.

The term “protein” or “polypeptide” is used here to indicate an organic polymer composed of two or more amino acid monomers and/or analogs thereof. As used herein, the term “amino acid” refers to any natural and/or synthetic amino acids. Accordingly, the term polypeptide includes amino acid polymers of any length, including full length proteins and peptides, as well as analogs and fragments thereof.

The term “enzyme” refers to any substance that catalyzes of promotes any chemical or biochemical reaction. Enzymes are totally or partially composed by polypeptides, but can include molecules composed of a different molecule, including nucleic acids.

The term “domain”, “protein domain” or “enzyme domain” refers to a distinct structural unit of a protein or polypeptide, where a specific reaction takes place or where a specific function can be attributed. A protein or enzyme may possess one or more domains that may have separate functions and may fold as independent compact units.

The term “E-value” or “expected value” refers to a parameter that describes the number of hits one can expect to see by chance when searching a Conserved Domain Database from National Center for Biotechnology Information (http://www.ncbi.nlm.nih.gov/cdd).

The term “pathway” or “metabolic pathway” is used here to refer to a biological process including one or more enzymatically controlled chemical reactions by which a substrate is converted into a product. Accordingly, a pathway for the convertion of a carbon source into n-propanol is a biological process including one or more enzymatically controlled reactions by which the carbon source is converted to n-propanol. A “heterologous pathway” refers to a pathway in which at least one or more chemical reactions of the pathway is catalyzed by at least one heterologous enzyme. On the other hand, a “native pathway” refers to a pathway wherein all chemical reactions are catalyzed by a native enzyme.

The term “reducing equivalents in the form of NAD(P)H”, refers to the coenzymes nicotinamine adenine dinucleotide (NAD) or nicotinamine adenine dinucleotide phosphate (NADP) in their reduced forms. In the reduced forms, these coenzymes are able to donate their electrons, or reducing equivalents, for reduction reactions catalyzed by enzymes that use these coenzymes as co-factors, such as the enzymes of the class of oxidoreductases.

The term “microorganism extract” or “yeast extract” or “Propionibacterium spp. extract” are used here to refer a water-soluble portion of autolyzed microorganism cell culture, like yeast or Propionibacterium spp.

The microorganism extract is typically prepared by growing the microorganism in a carbohydrate-rich medium. After that the microorganism is harvested, washed, resuspended in water and submit to an autolysis process (self-digestion of the cell wall using the enzymes). The microorganism extract is the total soluble portion of this autolytic action.

The terms “heterologous” or “exogenous” are used here to refer to enzymes and nucleic acids that are expressed in other organism different than that from which they were originated, independently on the level of expression, which can be lower, equal, or higher than the level of expression of the molecule found in the native microorganism.

The terms “endogenous” or “native” are used here to refer to enzymes and nucleic acids that are expressed in the organism in which they are found in nature, independently of their level of expression.

The terms “host” or “host cells” are used here interchangeably to refer to microorganisms, native or wild type, eukaryotic or prokaryotic, that can be engineered for the conversion of a carbon source to n-propanol. The terms host and host cell refers not only to the particular subject cell but also to the progeny or potential progeny of such cell, carrying the genetic modifications. Since certain modifications may occur in this progeny due to mutation or environmental difference, it is possible that such progeny may not be identical to the parent cell, but are still included within the scope of the term as used here.

The term “yield” as used herein refers to the amount of product obtained from the amount of substrate in g/g.

The microorganisms disclosed herein can be wild-type microorganisms or engineered using genetic engineering techniques to provide microorganisms that utilize heterologously or endogenously expressed enzymes to produce n-propanol and, optionally, iso-propanol and/or ethanol at high carbon yield. The terms “modified” or “modification” as used here refer to the state of a metabolic pathway being altered in which at least one step or process in the pathway is either increased (upregulated) or decreased (downregulated), such as an activity of an enzyme or expression of a nucleic acid. In a specific embodiment, the modification is the result of an alteration in a nucleic acid sequence which encodes as enzyme in the pathway, an alteration in expression of a nucleic acid sequence which encodes an enzyme in the pathway, or an alteration in translation or proteolysis of an enzyme in the pathway (i.e. alcohol dehydrogenase), or a combination thereof. A skilled artisan recognizes that there are commonly used methods in the art to obtain alterations, such as by deletion or superexpression.

The term “mediator” includes any molecules with the characteristics of being lipid or water soluble, pH-independent, stable and holding a redox potential for driving the electron transfer process.

The term “electrode” includes any electrically conductive material, preferably graphite or a noble metal. One or more reference electrodes can be included in the system.

The production of propionic acid from glucose by several species of bacteria, such as Propionibacterium acidipropionici, Propionibacterium acnes, Propionibacterium freudenreichii and Selenomonas ruminantium, can be accomplished by the following series of steps. This series is representative of a number of pathways known to those skilled in the art. Glucose is converted in a series of steps by enzymes of glycolytic pathway to pyruvate. The pyruvate may be converted to Acetyl-CoA and then to acetate or to propionic acid through the dicarboxylic acid cycle, which may include the following conversion steps:

Conversion a) Pyruvate and Methylmalonyl-CoA to Oxaloacetate and Propionyl-CoA through the action of the enzyme methylmalonyl-CoA carboxytransferase (E.C. 2.1.3.1). A methylmalonyl-CoA carboxytransferase can have an amino acid sequence corresponding to SEQ ID NO: 2, 4, 6, 8, 10, 12, 14, 16, or 18, which can be encoded by the corresponding nucleic acid sequences set forth in SEQ ID NOs: 1, 3, 5, 7, 9, 11, 13, 15 or 17, respectively;

Conversion b) Oxaloacetate and NADH to Malate and NAD⁺ through the action of the enzyme malate dehydrogenase (E.C. 1.1.1.37). A malate dehydrogenase can have an amino acid sequence corresponding to SEQ ID NO: 20, 22, 24, or 26, which can be encoded by the corresponding nucleic acid sequences set forth in SEQ ID NOs: 19, 21, 23 or 24 respectively;

Conversion c) Malate to Fumarate and H₂O through the action of the enzyme fumarate hydratase (E.C. 4.2.1.2). A fumarate hydratase can have an amino acid sequence corresponding to SEQ ID NO: 28, 30, 32, 34, 36, which can be encoded by the corresponding nucleic acid sequences set forth in SEQ ID NOs: 27, 29, 31, 33, 35, respectively;

Conversion d) Fumarate and FPH₂ to Succinate and FP through the action of the enzyme succinate dehydrogenase (E.C. 1.3.99.1). A succinate dehydrogenase can have an amino acid sequence corresponding to SEQ ID NO: 38, 40, 42, 44, 46, 48, 50, 52, 54, 56, 58 or 60, which and be encoded by the corresponding nucleic acid sequences set forth in SEQ ID NOs: 37, 39, 41, 43, 45, 47, 49, 51, 53, 55, 57, or 59, respectively;

Conversion e) Succinate and Propionyl-CoA to Succinyl-CoA and Propionate through the action of the enzyme propionyl-CoA: succinate CoA transferase (E.C. 2.8.3). A propionyl-CoA: succinate CoA transferase can have an amino acid sequence corresponding to SEQ ID NO: 62, 64, 66, or 68, which can be encoded by the corresponding nucleic acid sequences set forth in SEQ ID NOs: 61, 63, 65 or 67, respectively;

Conversion f) Succinyl-CoA to (S)Methylmalonyl-CoA through the action of the enzyme methylmalonyl-CoA mutase (E.C. 5.4.99.2). A methylmalonyl-CoA mutase can have an amino acid sequence corresponding to SEQ ID NO: 70, 72, 74, 76, 78, 80, 82 or 84, which can be encoded by the corresponding nucleic acid sequences set forth in SEQ ID NOs: 69, 71, 73, 75, 77, 79, 81 or 83, respectively;

Conversion g) (S)Methylmalonyl-CoA to (R)Methylmalonyl-CoA through the action of the enzyme methylmalonyl-CoA epimerase (E.C. 5.1.99.1). A methylmalonyl-CoA epimerase can have an amino acid sequence corresponding to SEQ ID NO: 86, 88, 90, 92, which can be encoded by the corresponding nucleic acid sequences set forth in SEQ ID NOs: 85, 87, 89 or 91, respectively; and

Conversion h) (R)Methylmalonyl-CoA and Pyruvate to Propionyl-CoA and Oxaloacetate through the action of the enzyme methylmalonyl-CoA carboxytransferase (E.C. 2.1.3.1), thus closing the cycle.

Natural or recombinant microorganisms containing the genes coding for the enzymes catalyzing the conversions a, b, c, d, e, f, g and h may be isolated or constructed using techniques such as heterologous DNA insertion, differential expression or deletion of genes well known by those skilled in the art. Alternatively, any genes encoding the enzymes catalyzing the conversions a, b, c, d, e, f, g and h that are known in the art can be used in the methods disclosed herein.

In some organisms, the production of alcohols from their acyl-CoA intermediates occurs in a two-step process through the sequential action of an aldehyde dehydrogenase and an alcohol dehydrogenase, with both steps being dependent on reducing equivalents in the form of NAD(P)H. Examples of aldehyde dehydrogenases that act on the acyl-CoA intermediates include, but are not limited to the ones found in Mus musculus (GenBank Accession No. AC162458.4) (SEQ ID NO.: 94, encoded by SEQ ID NO.: 93); Clostridium botulinum A str. ATCC No. 3502 (American Type Culture Collection or “ATCC”, P.O. Box 1549, Manassas, Va. USA, (GenBank Accession No. AM412317.1)_(SEQ ID NO.: 96, encoded by SEQ ID NO.: 95); Saccharomyces cerevisiae (GenBank Accession No. EU255273.1) (SEQ ID NO.: 98, encoded by SEQ ID NO.: 97). Yet in other microorganisms, the production of alcohols occurs only through the acyl-CoA intermediate of the organic acid in two sequential steps catalyzed by similar aldehyde and alcohol dehydrogenase enzymes, dependent on reducing equivalents in the form of NAD(P)H. Examples of aldehyde dehydrogenase that act on acyl-CoA intermediates include, but are not limited to, Rhodococcus opacus (GenBank Accession No. AP011115.1) (SEQ ID NO.: 100, encoded by SEQ ID NO.: 99), Entamoeba dispar (GenBank Accession No. DS548207.1) (SEQ ID NO.: 102, encoded by SEQ ID NO.: 101) and Lactobacillus reuteri (GenBank Accession No. ACHG01000187.1) (SEQ ID NO.: 116, encoded by SEQ ID NO.: 115). Examples of alcohol dehydrogenases that catalyze the conversion of an aldehyde to its corresponding primary alcohol include, but are not limited to, Aspergillus niger (GenBank Accession No. AM269994.1) (SEQ ID NO.: 104, encoded by SEQ ID NO.: 103), Streptococcus pneumoniae Taiwan19F-14 (GenBank Accession No. CP000921.1) (SEQ ID NO.: 106, encoded by SEQ ID NO.: 105) and Salmonella enterica (GenBank Accession No. CP001127.1) (SEQ ID NO.: 108, encoded by SEQ ID NO.: 107). Yet in other microorganisms, both reactions can occur sequentially by the action of a single enzyme possessing both aldehyde/alcohol dehydrogenase domains, independently of the enzyme having only these two domains or more. Examples of such multifunctional enzymes include, but are not limited to, Lactobacillus sakei (GenBank Accession No. CR936503.1) (SEQ ID NO.: 118, encoded by SEQ ID NO.: 117), Giardia intestinalis (GenBank Accession No. U93353.1) (SEQ ID NO.: 120, encoded by SEQ ID NO.: 119), Shewanella amazonensis (GenBank Accession No. CP000507.1) (SEQ ID NO.: 122, encoded by SEQ ID NO.: 121), Thermosynechococcus elongatus (GenBank Accession No. BA000039.2) (SEQ ID NO.: 124, encoded by SEQ ID NO.: 123), Clostridium acetobutylicum (GenBank Accession No. AE001438.3) (SEQ ID NO.: 126, encoded by SEQ ID NO.: 125) and Clostridium carboxidivorans ATCC No. BAA-624T (GenBank Accession No. ACVI01000101.1) (SEQ ID NO.: 128, encoded by SEQ ID NO.: 127).

Examples of enzymes that can be used in the present inventions include, but not limited to, those enzymes listed in the Tables 1-3.

TABLE 1
Aldehyde Dehydrogenases that Can Use Acyl-CoA Intermediates as a
Substrate

| Organism | GenBank Accession No. | GI number |
|---|---|---|
| Rhodococcus opacus | AP011115.1 | 226243131 |
| Entamoeba dispar | DS548207.1 | 165903565 |
| Lactobacillus reuteri | ACHG01000187.1 | 227184849 |
| Mus musculus | AC162458.4 | 7106242 |
| Clostridium botulinum A | AM412317.1 | 148288571 |
| str. ATCC No. 3502 | | |
| Saccharomyces cerevisiae | EU255273.1 | 160415767 |

TABLE 2
Aldehyde Dehydrogenases that Catalyze the Conversion of an
Aldehyde to its Corresponding Primary Alcohol

| Organism | GenBank Accession No. | GI number |
|---|---|---|
| Aspergillus niger | AM269994.1 | 145231224 |
| Streptococcus pneumoniae | CP000921.1 | 225728188 |
| Taiwan19F-14 | | |
| Salmonella enterica | CP001127.1 | 194710780 |

TABLE 3
Aldehyde/Alcohol Dehydrogenases Multifunctional Enzymes

| Organism | GenBank Accession No. | GI number |
|---|---|---|
| Lactobacillus sakei | CR936503.1 | 78609634 |
| Giardia intestinalis | U93353.1 | 2052472 |
| Shewanella amazonensis | CP000507.1 | 119767329 |
| Thermosynechococcus | BA000039.2 | 22293948 |
| elongatus | | |
| Clostridium | AE001438.3 | 14994351 |
| acetobutylicum | | |
| Clostridium | ACVI01000101.1 | 255508861 |
| carboxidivorans ATCC | | |
| No. BAA-624T | | |

Natural or recombinant organisms containing the gene that encodes the enzyme alcohol/aldehyde dehydrogenase capable of reducing an acyl-CoA or an organic acid and then the aldehyde or a ketone to the corresponding primary alcohol may be isolated or constructed using techniques such as heterologous DNA insertion, differential expression or deletion of genes well known in the art.

Acyl-CoA+NAD(P)H+H⁺⇄Aldehyde+NAD(P)⁺ or Conversion ia)

Organic acid+NAD(P)H+H⁺⇄Aldehyde+NAD(P)⁺+H₂O and Conversion ib)

Aldehyde or ketone+NAD(P)H+H⁺⇄alcohol+NAD(P)⁺ Conversion j)

In order to maximize the production of n-propanol, it is of great importance that the carbon flux of our engineered microorganism flows preferentially from pyruvate to propionic acid through the dicarboxylic acid cycle. However, the present invention realizes that due to cellular requirements for ATP and NAD(P)H some of the carbon might flow to the production of acetate from pyruvate through an irreversible oxidative decarboxylation reaction. The acetate or acetyl-CoA intermediate thus formed are of no economic interest. However, this acetate or its acetyl-CoA intermediate may be further metabolized into ethanol by the action of the enzymes aldehyde/alcohol dehydrogenases described above, or alternatively, these intermediates could be further metabolized into isopropanol by the condensation of two molecules of acetyl-CoA into acetoacetyl-CoA and CoA, followed by another oxidative decarboxylation reaction into acetone and final reduction into isopropanol, through the action of the enzymes from the isopropanol production pathway of Clostridium beijerinckii, as disclosed in International Application No. WO 2008/131286 A1.

Conversion k) condensation of the two molecules of acetyl-CoA into acetoacetyl-CoA and CoA through the action of the enzyme thiolase (E.C. 2.3.1.9). A thiolase can have an amino acid sequence corresponding to SEQ ID NO: 142 or 143;

Conversion l) acetoacetyl-CoA into acetoacetate and CoA through the action of the enzyme acetoacetyl-CoA hydrolase (E.C. 3.1.2.11). An acetoacetyl-CoA hydrolase can have an amino acid sequence corresponding to SEQ ID NO: 140 or 141;

Conversion m) decarboxylation of acetoacetate into acetone through the action of the enzyme acetoacetate decarboxylase (E.C. 4.1.1.4). An acetoacetate decarboxylase can have an amino acid sequence corresponding to SEQ ID NO: 132, 133, 134, 135, 136 or 137;

Conversion n) reduction of acetone into isopropanol through the action of the enzyme primary-secondary alcohol dehydrogenase (E.C. 1.1.1.1) found in microorganisms such as Clostridium beijerinckii (SEQ ID NO.: 114, encoded by SEQ ID NO.: 113), Burkholderia spp (for example, B. xenovorans SEQ ID NO.: 110, encoded by SEQ ID NO.: 109). and Thermoanaerobacter brockii (SEQ ID NO.: 112, encoded by SEQ ID NO.: 111).

In certain embodiments, the engineered microorganism will express the enzymes corresponding to the conversions a, b, c, d, e, f, g, h, ia, ib and j, in which at least one of the conversions is carried out by an heterologous gene, and the final end alcohol products of the fermentation are either n-propanol or ethanol or a mixture of both.

In certain embodiments, the engineered microorganisms will express the enzymes corresponding to the conversions a, b, c, d, e, f, g, h, ia, ib, j, k, l, m, and n, in which at least one of the conversions is carried out by an heterologous gene, and the final end alcohol products of the fermentation are either n-propanol, ethanol or isopropanol or a mixture thereof.

In certain embodiments, the gene encoding for an enzyme acetate kinase (E.C. 2.7.2.1) of the host organism, catalyzing the conversion of acetyl-CoA into acetate, will have its expression altered so as to diminish its activity and thus increase availability of acetyl-CoA for isopropanol production. An acetate kinase can have an amino acid sequence corresponding to SEQ ID NO.: 139 and can be encoded by the nucleic acid sequence set forth in SEQ ID NO.: 138. For example, the acetate kinase encoding gene of P. acidipropionici (GenBank Accession No. AY936474.1) may be altered, deleted or underexpressed using techniques known by those skilled in the art.

The invention encompasses the use of isolated or substantially purified polynucleotide and enzyme or protein compositions. An “isolated” or “purified” polynucleotide or enzyme, or biologically active portion thereof, is substantially or essentially free from components that normally accompany or interact with the polynucleotide or protein as found in its naturally occurring environment. Thus, an isolated or purified polynucleotide or enzyme is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. An enzyme or protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and enzymes encoded thereby are also encompassed by the present invention. By “fragment” is intended a portion of the polynucleotide or a portion of the amino acid sequence and hence enzyme or protein encoded thereby. Fragments of polynucleotides comprising coding sequences may encode enzyme or protein fragments that retain biological activity of the native enzyme. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

A fragment of a polynucleotide that encodes a biologically active portion of an enzyme of the invention will encode at least 15, 25, 30, 50, 100, 150, 200, 300, 400, 500, 750. or 1000 contiguous amino acids, or up to the total number of amino acids present in a full-length enzyme of the invention. Fragments of a polynucleotide encoding an enzyme of the present invention that are useful as hybridization probes or PCR primers generally need not encode a biologically active portion of the enzyme.

Thus, a fragment of polynucleotide of the present invention may encode a biologically active portion of an enzyme, or it may be a fragment that can be used as a hybridization probe or PCR primer using methods disclosed below. A biologically active portion of an enzyme protein can be prepared by isolating a portion of one of the polynucleotides of the invention, expressing the encoded portion of the enyzme or protein (e.g., by recombinant expression in vivo), and assessing the enzyme activity of the encoded portion of the enzyme. Polynucleotides that are fragments of a nucleotide sequence comprise at least 16, 20, 50, 75, 100, 150, 200, 250, 300, 350, 400, 450, 500, 550, 600, 650, 700, 800, 900, 1000, 1100, 1200, 1300, 1400, 1500, 2000, 2500, or 3000 contiguous nucleotides, or up to the number of nucleotides present in a full-length polynucleotide disclosed herein.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode an enzyme of the invention. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired biological activity of the native protein. The biological activity of variant proteins of the invention can be assayed by methods known in the art. Such variants may result from, for example, genetic polymorphism or from human manipulation. Biologically active variants of a native enzyme of the invention will have at least about 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired biological activity of the native protein. The biological activity of variant proteins of the invention can be assayed by methods known in the art. Such variants may result from, for example, genetic polymorphism or from human manipulation. Biologically active variants of a native enzyme aldehyde dehydrogenase and alcohol dehydrogenase of the invention will have an E-value threshold below 1e-2 when compared with conserved domain protein database (CDD) from National Center for Biotechnology Information (http://www.ncbi.nlm.nih.gov/cdd).

The enzymes or proteins of the invention may be altered in various ways including amino acid substitutions, deletions, truncations, and insertions. Methods for such manipulations are generally known in the art. For example, amino acid sequence variants and fragments of the enzymes can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired enzyme activity. Obviously, the mutations that will be made in the DNA encoding the variant must not place the sequence out of reading frame and optimally will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, enzyme activity can be evaluated by routine assays known in the art.

Variant polynucleotides and enzymes also encompass sequences and enzymes derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

It is recognized that the methods of the present invention encompass the use of polynucleotide molecules and proteins comprising a nucleotide or an amino acid sequence that is sufficiently identical to a nucleotide or amino acid sequence disclosed herein. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 90%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTn and BLASTx programs of Altschul et al. (1990) J. Mol. Biol. 215:403. BLAST nucleotide searches can be performed with the BLASTn program, score=100, wordlength=12, to obtain nucleotide sequences homologous to the polynucleotide molecules of the invention. BLAST protein searches can be performed with the BLASTx program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLAS Tx and BLASTn) can be used. See http://www.ncbi.nlm nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, Md., USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website: http://www.ebi.ac.uk/Tools/clustalw/index.html). In certain embodiments, any genes encoding for enzymes with one or more of the aldehyde dehydrogenase and alcohol dehydrogenase activities may be used. These enzymes may be wild-type enzymes from a different organism, or may be artificial, recombinant or engineered enzymes.

In certain embodiments, the metabolic reactions described within this invention may be catalyzed by one or more enzymes regardless of the number of steps catalyzed by each enzyme which may be single or multi-functional and still be included within the scope of this invention.

In certain embodiments, any genes encoding for enzymes with the same activity as any of the enzymes described within this invention may be used. These enzymes may be wild-type enzymes from a different organism, or may be artificial, recombinant or engineered enzymes.

Due to the inherent degeneracy of the genetic code, other nucleic acid sequences which encode substantially the same or a functionally equivalent amino acid sequence can also be used to express such enzymes. As will be understood by those of skill in the art, it can be advantageous to modify a coding sequence to enhance its expression in a particular host. The codons that are utilized most often in a species are called “optimal codons”, and those not utilized very often are classified as “rare or low-usage codons”. Codons can be substituted to reflect the preferred codon usage of the host, a process sometimes called “codon optimization” or “controlling for species codon bias”. Expression of genes is a complex mechanism that may be modified by molecular biology techniques. For example, expression of heterologous genes may be controlled by an inducible promoter or a constitutive promoter. The heterologous genes may either be integrated into a chromosome of the host or present as extra-chromosomal genetic elements (such as plasmids, BAC, YAC, etc.) that can be inherited by daughter cells. Such extra-chromosomal genetic elements may contain selection markers.

Methods for expressing polypeptide from an exogenous nucleic acid molecule include constructing a nucleic acid such that a regulatory element (promoter, enhancers and the like) promotes the expression of a nucleic acid sequence that encodes the desired polypeptide at a desired condition.

In another embodiment, heterologous control elements can be used to activate or repress expression of endogenous or heterologous genes. Moreover, when expression is to be repressed or eliminated, the gene for the relevant enzyme, protein or RNA can be eliminated, for example, by knock-out mutation obtained through homologous recombination or other known deletion techniques. The use of the technique of interference RNA (iRNA) for gene post-trascriptional silencing could also be used.

Methods that modify the expression of genes in microorganisms are contemplated for use in the construction of the microbial cells of the present invention.

Any method capable of introducing an exogenous nucleic acid molecule into microorganisms can be used. For example, electroporation, conjugation, heat shock, Agrobacterium tumefaciens mediated transformation, protoplasts fusion, etc.

The exogenous nucleic acid molecule contained within a microorganism described herein may be maintained within that cell in any form, i.e., these molecules can be integrated into the any chromosome or maintained in an extra-chromosomal state that can be passed on to daughter cells. Additionally, these microorganisms can be stably or transiently transformed. Moreover, exogenous nucleic acid molecule may be present as single or multiple copies into the host microorganism.

The reducing equivalents needed for the conversion of the propionate/propionyl-CoA intermediate into n-propanol may be supplied to the microorganism in vivo through the use of a recombinant NAD(P)H recycling system and the external supply of a formate salt.

According to the present invention, it is possible to drive redox balance artificially in three main ways. As example, one way is the introduction of a recombinant NAD(P)H and/or recycling system based on a the introduction of a gene coding for an enzyme that catalyzes the conversion of formate salt into CO₂ with the concomitant regeneration of the reduced form NAD(P)H and the external supply of formate to the growth medium. See, U.S. Patent Application Publication No. 2003/0175903 A1, herein incorporated by reference.

The reducing equivalents needed for the conversion of the propionate/propionyl-CoA intermediate into n-propanol may also be supplied by the addition of an overpressure of H₂ to the bioreactor (at low or high pressures, but preferentially at 1-2 atmospheres) as described in U.S. Pat. No. 4,732,855, herein incorporated by reference. This overpressure can be used in microorganism that express a hydrogenase enzyme, native or heterologous.

Another alternative is to supply the reducing equivalents needed for the conversion of the propionate/propionyl-CoA intermediate into n-propanol through the use of cathodes and a mediator molecule. This reaction occurs simultaneous to the fermentation process in a bioelectric reactor, where the mediator is a external molecule that has a function of transferring the electrons from a cathode to the electron carriers of the living cell (NAD(P)) as described by Thrash & Coates 2008, Environ. Sci. Technol. 42:3921-3931, herein incorporated by reference.

The working cathode can be poised at several potentials against the reference electrode, such as 10 mV, 100 mV, 200 mV, 400 mV, 600 mV and 800 mV or any potential value necessary to transfer electrons from the electrode to the growing cells. The cathodes can be constructed in different materials, shapes, sizes and superficial areas, such as single wires, nets or solid shape configurations. However, other shapes or configurations may be considered within the scope of the present invention.

The mediator molecule can be any molecule externally supplied or internally secreted and can be present at several concentrations, such as 0.2 mM, 0.4 mM, 0.6 mM, 0.8 mM, 1.0 mM, or any concentration necessary to transfer the electrons from the electrode to the cell with high performance and with the object of maximizing the concentration of interesting end-products and minimizing the electrical current generated during this process. Examples of suitable mediators for this process are benzyl viologen, methyl viologen, anthraquinone 2,6-disulfonic acid, neutral red and cobalt sepulchrate. Other suitable mediator molecules for the process of the present invention are compounds present in yeast extract and endogenous mediator present in Propionibacterium spp. extract. Another embodiment of the invention is the use of endogenous mediator by recirculation of the cells to the bioreactor.

In the present invention, the preferred form for externally supplying reducing equivalents to the culture medium is through the use of electrodes and a mediator molecule.

The electrical current used to supply the electrodes can be originated by renewable or non-renewable energy sources. However, the preferred source is a renewable source, such as hydroelectrical plants or, more preferentially according to the biorefinery concepts, such as through the burning of sugarcane bagasse.

The bioelectrical reactor uses a two or three electrode system for precise measurement and control of the potential at the working electrode (cathode) and the auxiliary counter electrode (anode). If necessary by the reactor configuration an electron shuttle may be used. Any kind of reference electrode system known at the state of the art as adequate for aqueous media, as the hydrogen electrode or the silver chloride electrode, can be used by the present invention as a reference electrode when necessary.

The cathodic voltage should be maintained below 3.0 V, preferentially below to 1.5 V, to prevent the electrolysis of water what would undesirably increase the pH of the media and release gaseous hydrogen.

In addition, high concentrations of chloride ions must be avoided in the anodic compartment to prevent its oxidation that would undesirably form chlorine that would react with water to form hypochlorous acid, which would be very prejudicial to the growth and integrity of the microorganisms.

The anode and cathode were separated by a separator element selected among the ones known by the state of the art. The purpose of this separator is to permit only the passage of ions and electrical current and avoid, or at least reduce, the transfer of chemicals, as sugars, and metabolites across it. As examples of the separators adequate for the present invention are ceramics porous septums, fibery diaphragms and, preferably, solid permeable electrolytes as the cation-selective membranes known as permselective membrane, commercially designed as Nafion or similar.

The cathode compartment is the place where the culture medium is fed and the fermentation is conducted. Its composition, made mainly by water and soluble nutrients, substrates and metabolites, permits its use as a catholyte in addition to its ability to promote the cells growth and the fermentation development.

The anode compartment must be filled with an aqueous solution, stable to the anode potential and able to conduct electricity. It can be usually constituted by an aqueous buffer as a 100 mM sodium phosphate solution.

The electrodes could be assembled in many different configurations as single wires, bars, rods, nets, porous agglomerates, woven structures or solid or perforated foils or plates, with a smooth or a rough surface. In the case of the cathodes they are preferably used as the baffles to prevent the vortex in stirred bioelectrical reactors. In the case of the anodes they are preferably assembled in the wall of the bioelectrical reactors, separated by a permselective membrane.

Electrodes must be made of a material stable to the corrosion in the bioelectrical reactor operational conditions and that is a good electricity conductor. The anode must be preferably made of carbon, graphite, or metals or alloys as nickel, platinum, stainless steel or titanium. The cathode must be made of any material adequate for use as cathodes, such as graphite, glassy carbon, stainless steel, carbon steel or metals or alloys as nickel, iron, lead, titanium, commercially designed as monel, sanicro, 2RK65 or similar. Preferably the cathode material will be constituted by a metal or alloy of high hydrogen overpotencial as titanium, monel, sanicro, or 2RK65.

Fermentation media in the present invention contain suitable carbon sources to yield a high productivity of propionic acid by native or engineered microorganisms hosting the dicarboxylic acid pathway and the n-propanol producing pathway by native or engineered microorganims. This carbon sources can include monosaccharides such as glucose, fructose and xylose; oligosaccharides such as sucrose and lactose; polysaccharides such as starch, pectin, cellulose and hemicellulose, and lignocellulosic materials; fatty acids; succinate; lactate; acetate; glycerol and mixtures thereof. Also, it can include other carbon sources from renewable feedstocks of complex composition such as sugarcane juice, sugarcane molasses or acid or enzymatic hydrolysates of lignocellulosic materials. Waste materials such as whey or industrial glycerol waste waters can also be used.

In certain embodiments of the present invention glycerol, sucrose and the complex multi-component sugarcane juice or sugarcane molasses are preferentially used.

In addition to the appropriate carbon sources, the culture media may be provided by other macronutrients such as nitrogen, and micronutrients such as phosphorous, potassium, sodium, calcium, vitamins and essentials metallic cofactors, known to those skilled in the art, according to the requirements of the producing microorganism.

In certain embodiments, the carbon source can be preferentially supplied with at least one nitrogen source.

In certain embodiments, the preferred nitrogen source is yeast extract.

In certain embodiments, the preferred nitrogen source is N₂.

In certain embodiments vitamin B5 (pantothenic acid) is supplied to the culture medium with the object of increasing productivity. This panthotenic acid may be provided in pure form or as a crude extract by-product of fermentation by another organism.

The microorganisms, native or engineered, must be grown in conditions for high yield production of the compounds of interest. Suitable culture conditions will be considered. The microorganisms, native or engineered for propionic acid and subsequent n-propanol production, grow at temperatures ranging from 25° C. to 60° C., where temperatures 30° C. to 32° C. are preferred. Suitable pH ranges for the fermentation high production, are between pH 5 to pH 7.5, where pH 6.5 to 6.8 are preferred. Reaction may be performed under anaerobic, microaerobic, or aerobic conditions.

In certain embodiments, fermentation under anaerobic condition is preferred.

The fermentative process in the present invention can employ various fermentation operations modes. Batch mode fermentation is a close system where culture media and producer microorganism, set at the beginning of fermentation, don't have any more inputs except for the reagents for pH control, foam control and others required for process sustenance. The process described in the present invention can also be employed in Fed-batch or continuous mode.

The fermentative process can be performed in free cell culture and in immobilized cell culture. For immobilized cell cultures is contemplated the use of different material supports such as alginates, fibrous bed, argyle materials such as chrysotile, montmorillonite KSF and montmorillonite K-10. However, other methods of immobilization are considered here within the scope of the present invention.

In certain embodiments, the preferred condition is the use of immobilized cells.

The present invention may be practiced in several bioreactor configurations, such as stirred tank, bubble column, airlift reactor and other known to those skilled in the art.

The products, n-propanol and, eventually, iso-propanol and/or ethanol, can be extracted from the fermentation broth using processes well-known in the state-of-the-art, such as for the separation of ethanol from broth. These processes include distillation, reactive distillation, azeotropic distillation and extractive distillation. There is no need to remove the total amount of water in the media.

In addition, the alcohols n-propanol and iso-propanol and/or ethanol, obtained according to the present invention can be dehydrated together in the same reactor using operating conditions to yield high amounts of propylene and an amount of ethylene. In certain embodiment of the invention, reactor feed stream can be a mixture of n-propanol and iso-propanol and/or ethanol or a mixture of these alcohols with water. Ethylene can be purified to used as a copolymer with propylene.

The dehydration reaction occurs in the presence of catalyst such as alumina, silica-alumina, zeolites and other metallic oxides using temperatures ranging from 180° C. to 600° C., preferentially from 300° C. to 500° C. The reaction is conduced in an adiabatic or isothermal reactor, which can also be a fixed or a fluidized bed reactor.

The dehydration reaction of n-propanol and, eventually, iso-propanol and/or ethanol, can be optimized using residence time ranging from 0.1 to 60 seconds, preferentially from 1 to 30 seconds. Non converted alcohol can be recycled to the dehydration reactor.

The contaminants that are generated in the process are removed through a purification section that is traditionally used in this type of reaction. Propylene can be washed with pure water or caustic solution to remove acids compounds like carbon dioxide and/or can be fed into beds to absorb polar compounds like water and also to remove carbon monoxide. Alternatively, a distillation column can be used to separate higher hydrocarbons such as propane, butane, butylene and higher compounds. The separation of propylene and ethylene is made by the methods know in the state-of-the-art as cryogenic distillation. Polymer grade propylene is provided by the process of the present invention and has 100% of renewable carbon content.

Polypropylene and their copolymers of the present invention are produced by polymerization processes well-known in the state of art, which can be conduced via bulk polymerization process with temperatures ranging from 105° C. to 300° C., or via polymerization in suspension with temperatures ranging from 50° C. to 100° C. Alternatively polypropylene can be produced in a gas phase reactor in the presence of a polymerization catalyst such as Ziegler-Natta or metalocene catalysts with temperatures ranging from 60° C.-80° C.

The product obtained by the processes described in the present invention has 100% of biobased content contributing to reduce greenhouse gas emission, since at the end of its life there would no fossil carbon emissions if it is incinerated.

### Example 1

### Fermentation of Sugarcane Juice by Propionibacterium Acidipropionici

A native strain of Propionibacterium acidipropionici (ATCC No. 4875) was used to study propionic acid and n-propanol production using sugarcane juice as a carbon source. The bacterium was cultured in a medium containing 30% sugar cane juice diluted in water and supplemented with 1 g/L of yeast extract. At this dilution, the starting concentrations of sugars in diluted sugarcane juice medium were measured at 53 g/L of sucrose, 10.9 g/L of glucose and 7.4 g/L of fructose. The medium was sterilized at 121° C. and 1 kgf/cm² for 20 min prior to use.

Free-cell batch fermentation was conducted in a 2.5 L bioreactor (BioFlo 3000-New Brunswick) containing 2.0 L of the sterile medium inoculated with 20 g/l (wet weight) of the adapted cells of P. acidipropionici. The bioreactor temperature was maintained at 30° C. and the agitation speed at 100 rpm. Constant pH of 6.5 was automatically controlled by adding a 4M NaOH solution. Anaerobic conditions were maintained through the use of a N₂ atmosphere.

Batch fermentation was stopped after 114 h and the products were quantified through High Performance Liquid Chromatography coupled to a Refraction Index detector and using standards for the desired metabolites (Varian Chromatographer using a Aminex HPX-87H Organic Acid Column from Transgenomic, operating at room temperature and using 0.002 M H₂SO₄ as the eluent at a flux of 0.6 mL/min). Table 4 shows the final concentration of the products. As can be observed, no n-propanol is detected at the growth conditions used.

TABLE 4
Final product concentrations after 114 h of fermentation by
Propionibacterium acidipropionici (ATCC No. 4875) of sugarcane juice
media (see composition in text) under controlled conditions of
temperature, pH and agitation.

| Component | Concentration (g/L) |
|---|---|
| Propionic acid | 28.0 |
| Acetic acid | 9.6 |
| Succinic acid | 8.1 |
| n-Propanol | ND |

ND: Not detected

### Example 2

### Engineering Propionibacterium Acidipropionici for In Vivo N-Propanol Production Through the Heterologous Expression of a Propionyl-CoA Reducing Pathway

### Constructs:

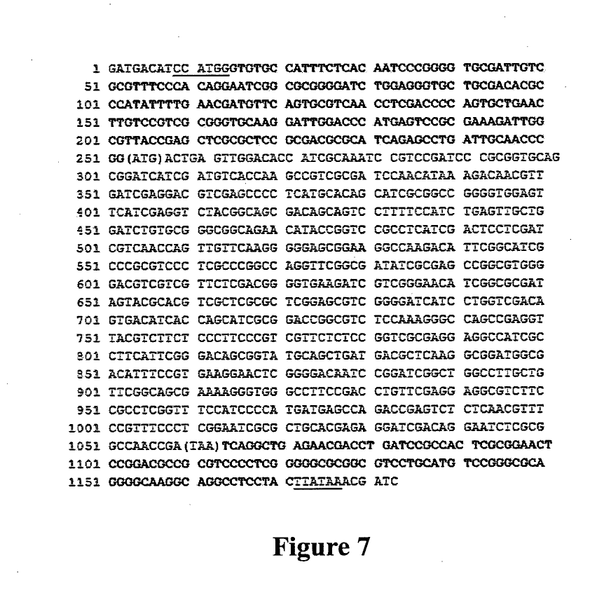

pBK1T. A shuttle plasmid, pBK1T, is constructed in two steps. First step consists of fusing a portion of the native pRGO1 plasmid of P. acidipropionici with a portion of a commercial pUC18 plasmid, as described by Kiatpapan et al. 2000 (Appl. Env. Microbiol. 66:4688-4695). As a result of this fusion, the plasmid has both origins of replication in E. coli and P. acidipropionici and the marker gene conferring resistance to ampicilin for E. coli; however, this resistance gene is not expressed in P. acidipropionici due to the differences in G+C content and codon usage. As an appropriate selection marker for P. acidipropionici, a synthetic construct was designed comprising a gene conferring resistance to the antibiotic thiostrepton, isolated from Streptomyces laurentii (GenBank Accession Number L39157.1) (SEQ ID NO.: 144), controlled by the promoter and terminator regions of the pa-mmc gene gene coding for the Methyl-malonyl CoA transcarboxilase (E.C. 2.1.3.1) of P. acidipropionici (SEQ ID NOs: 129, 130, 131). This synthetic construct is built by amplifying the thiostrepton resistance gene from plasmid pIJ680 (Hopwood et al., 1985, “Genetic manipulation of Streptomyces—A Laboratory Manual”, John Innes Foundation, Norwich) using adapter-primers PMMC_TSR-F (5′-CCGGGTTGCAATCAGGCTCTGATGCGCATGACTGAGTTGGACACCAT CG-3′) (SEQ ID NO.: 145) and TAPH_TSR-R (5′-TCAGGCTGAGAACGACCTGATCCGCCATTATCGGTTGGCCGCGAGAT-3′) (SEQ ID NO.: 146), in which the Forward primer contains a hybridization tail for fusing with the promoter region (underlined) and the Reverse primer contains a hybridization tail for fusing with the terminator region (underlined). The promoter and terminator regions of the pa-mmc gene of P. acidipropionici are PCR amplified from genomic DNA using the primers NcoI PMMC-F (5′-GATGACATCCATGGGTGTGCCATTTCTCACAATCC-3′) (SEQ ID NO.: 147), PMMC-R (5′-CCGGGTTGCAATCAGGCT CTGATGCGC-3′) (SEQ ID NO.: 148), TMMC-F (5′-TCAGGCTGAGAACGACCTGAT-3′) (SEQ ID NO.: 149) and PsiI_TMMC-R (5′-GATCGTTTATAAGTAGGAGGCCTGCCTTGC-3′) (SEQ ID NO.: 150). Both amplicons are joined together by single-joint PCR according to Yu et al., 2004 (Fungal Genetics and Biology 41:973-981). The sequence of the resulting synthetic construct (SEQ ID NO.: 151) is provided in FIG. 7. This is digested with NcoI and PsiI and inserted at the Psil (blunt) and NcoI sites of the fusion vector in order to create our shuttle vector pBK1T.

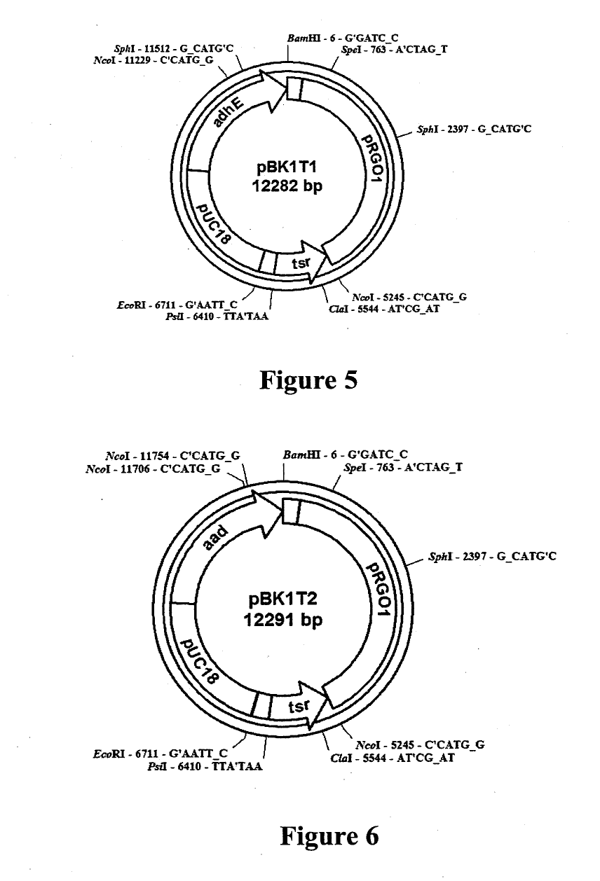

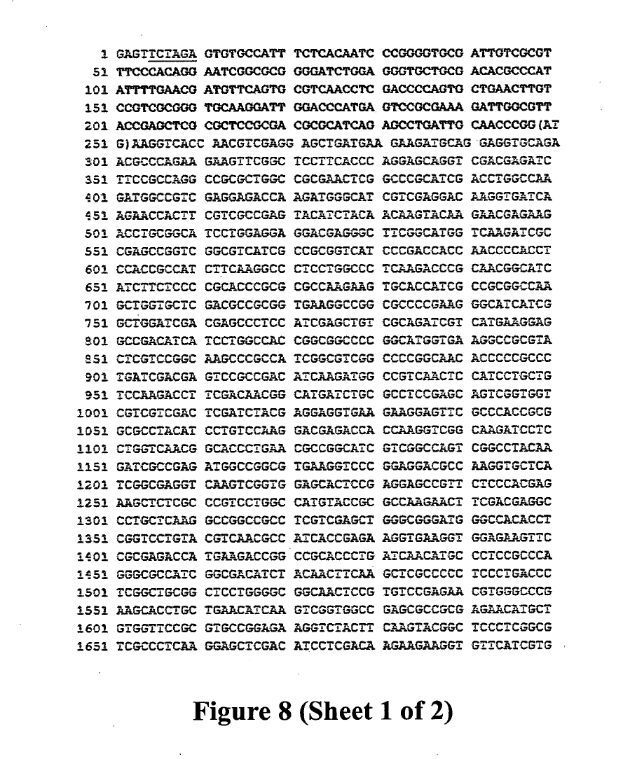

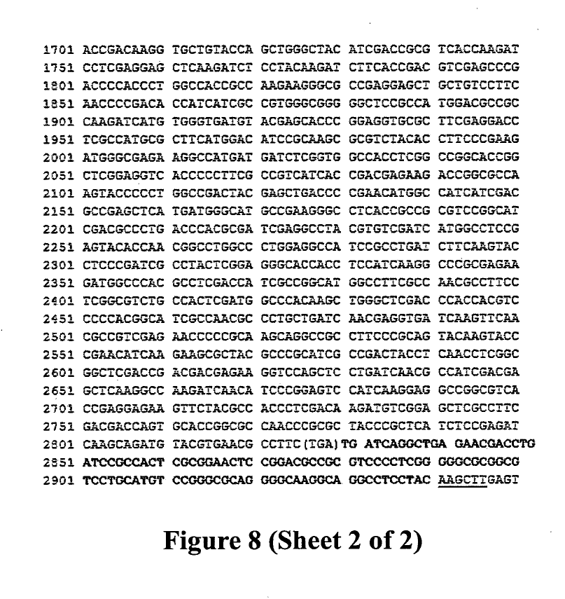

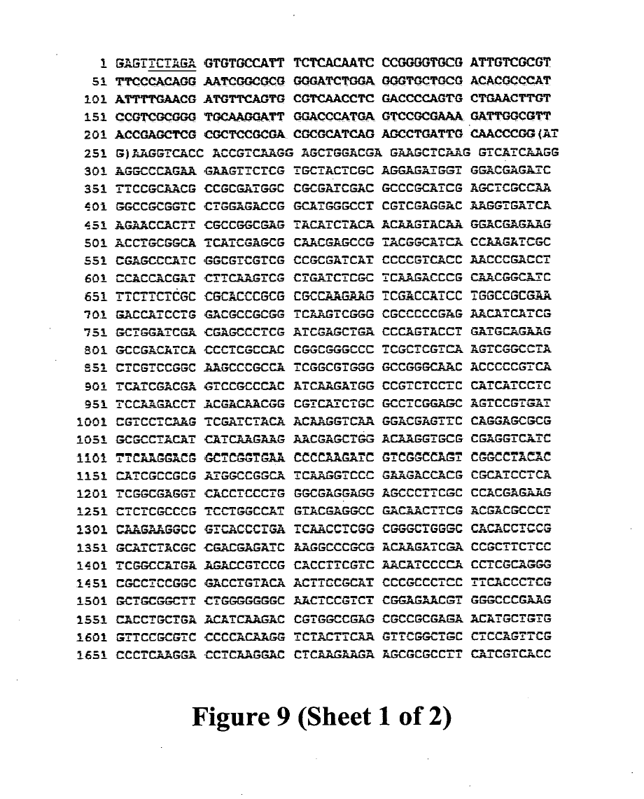

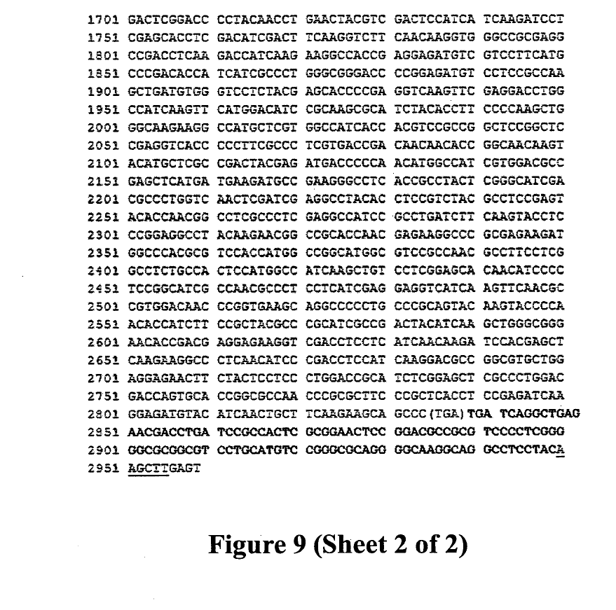

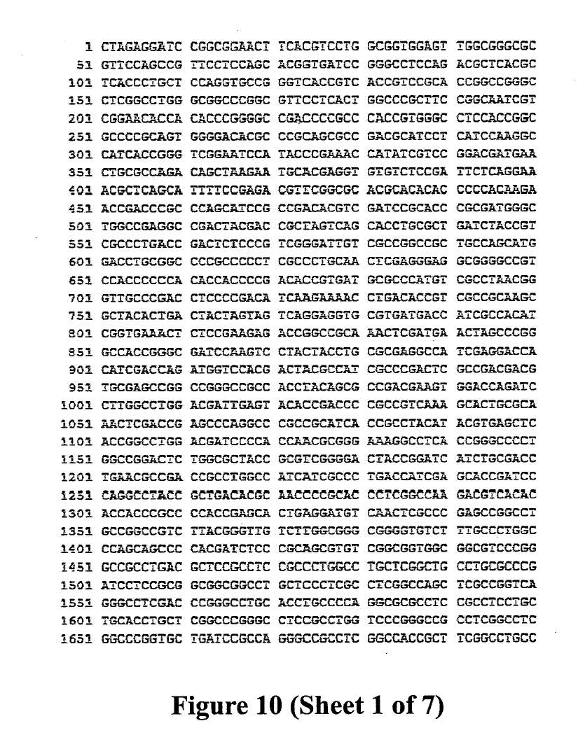

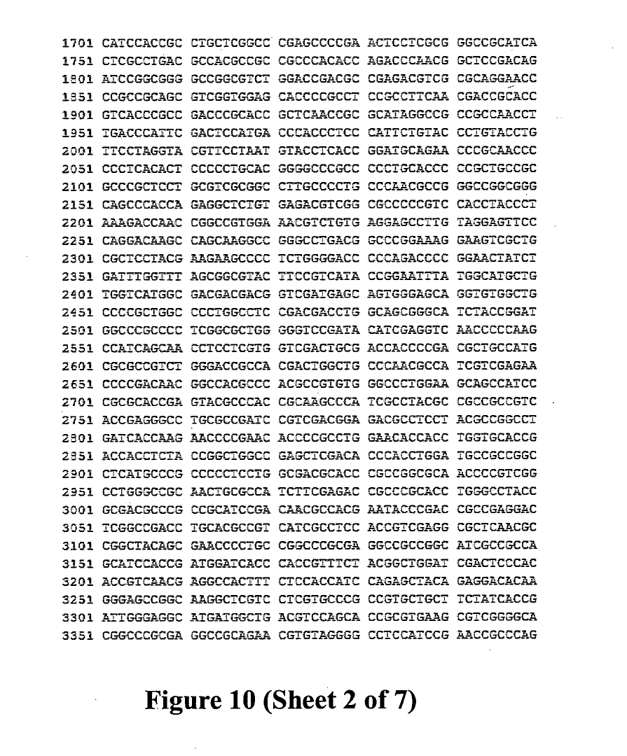

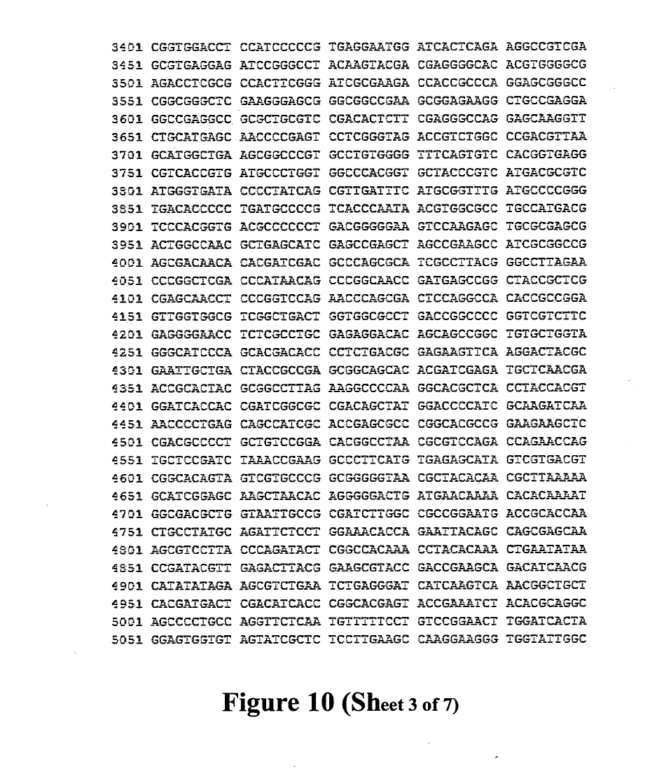

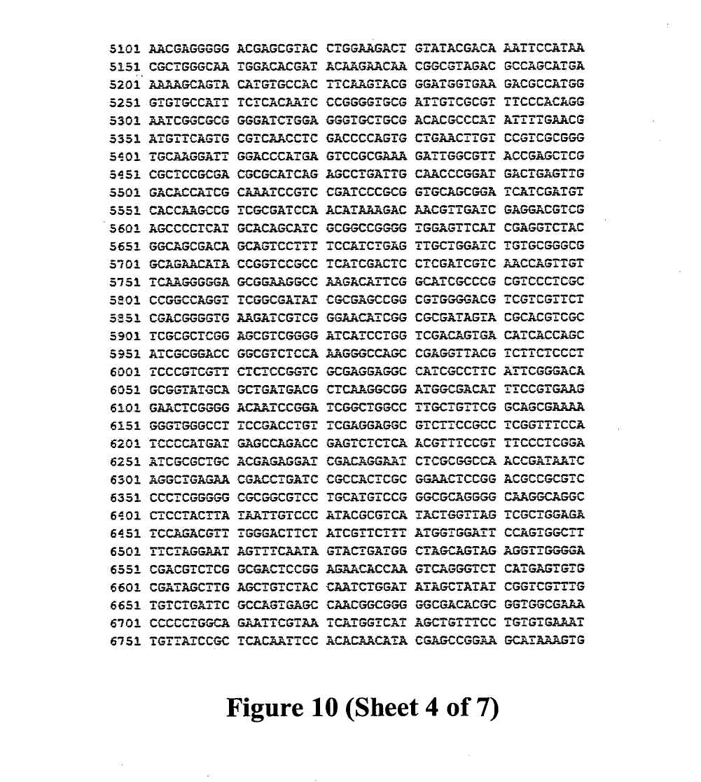

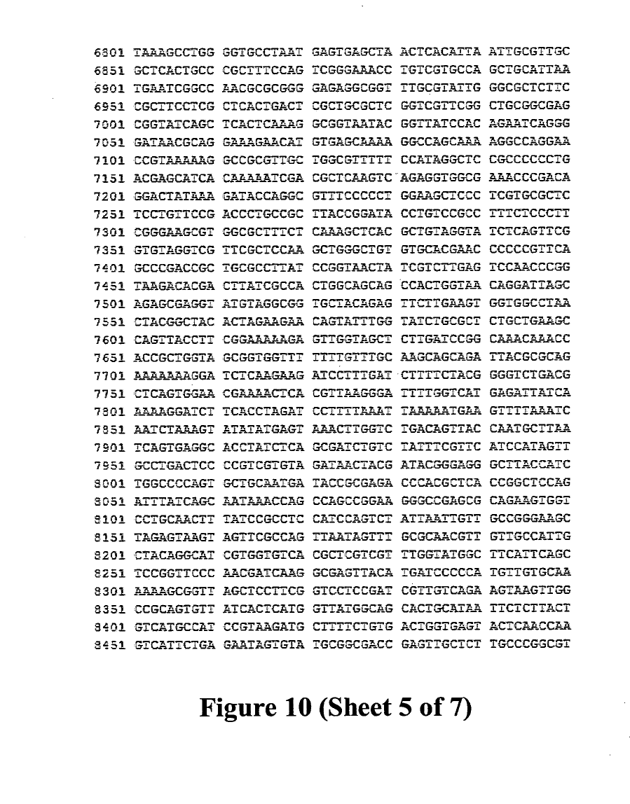

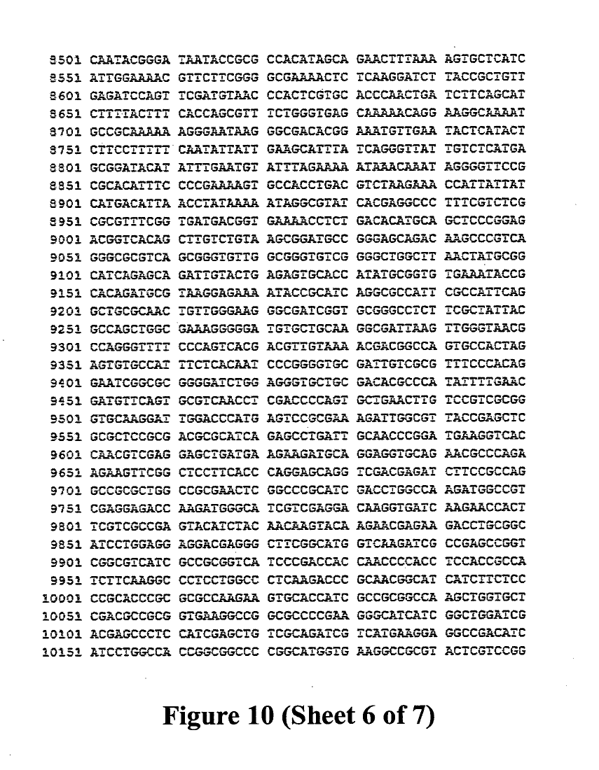

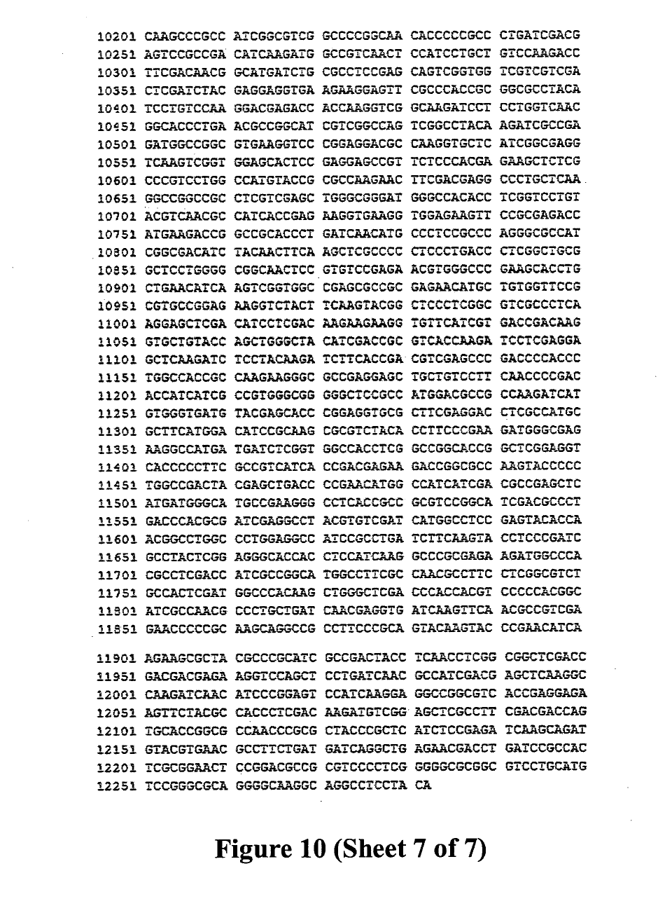

pBK1T1. Expression plasmid pBK1T1 is constructed by inserting into pBK1T a gene coding for the bifunctional aldehyde/alcohol dehydrogenase of Clostridium carboxidivorans (ATCC No. BAA-624T) (Uniprot Accession No. C6PZV5), controlled by the promoter and terminator regions of the gene coding for the Methyl-malonyl CoA transcarboxilase (E.C. 2.1.3.1) of P. acidipropionici. Due to differences in the G+C content and codon usage between P. acidipropionici and C. carboxidivorans, said gene was designed by reverse translation of the primary amino acid sequence. For this, a codon table is generated from host ribosomal protein genes, which are highly expressed. The codons are selected to resemble this table and the overall host G+C content, avoiding recognition sites of host restriction enzymes. Inverted repeats were also avoided to disrupt mRNA secondary structures. Finally, adaptors for digestion with the restriction enzymes XbaI and HindIII are added to the 5′ and 3′ ends of this sequence, respectively. The sequence of this synthetic construct (SEQ ID NO.: 152) is provided in FIG. 8. The designed 2950 bp construct, containing the gene, its controlling regions and cloning adaptors is synthesized by Epoch Life Science (http://epochlifescience.com/Service/Gene Synthesis.aspx). The construct is then digested and cloned into the XbaI and HindIII sites of pBK1T to generate the expression shuttle plasmid pBK1T1. A schematic view of this plasmid is provided in FIG. 5 and its sequence in (SEQ ID NO.: 154) FIG. 10.

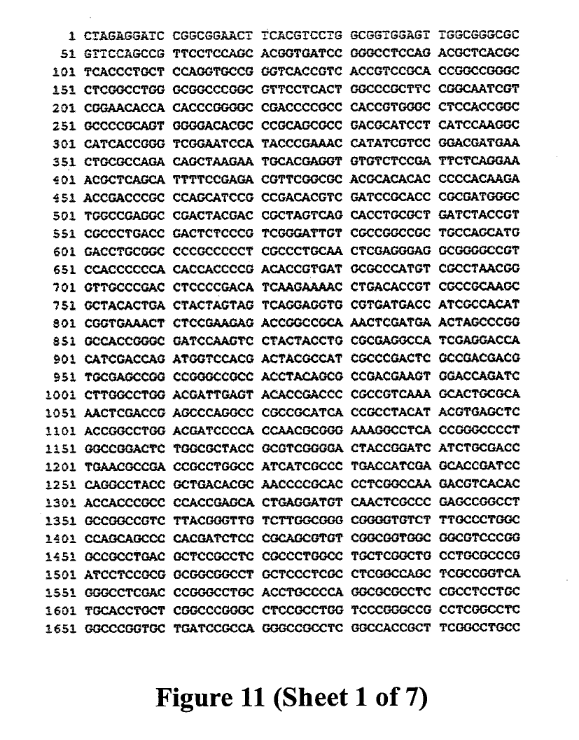

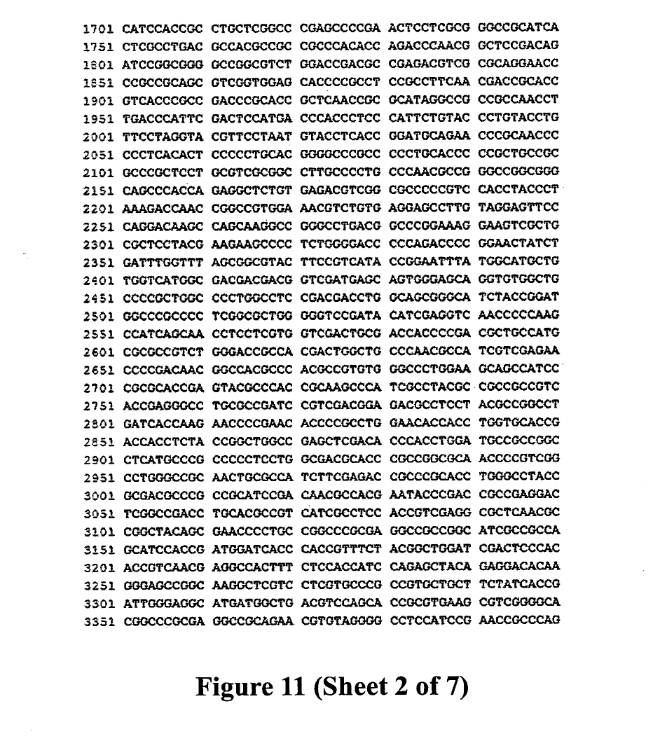

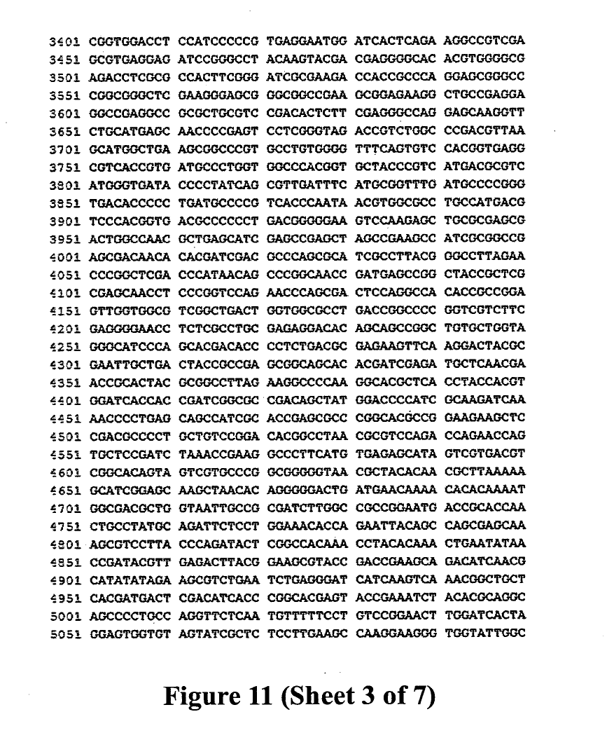

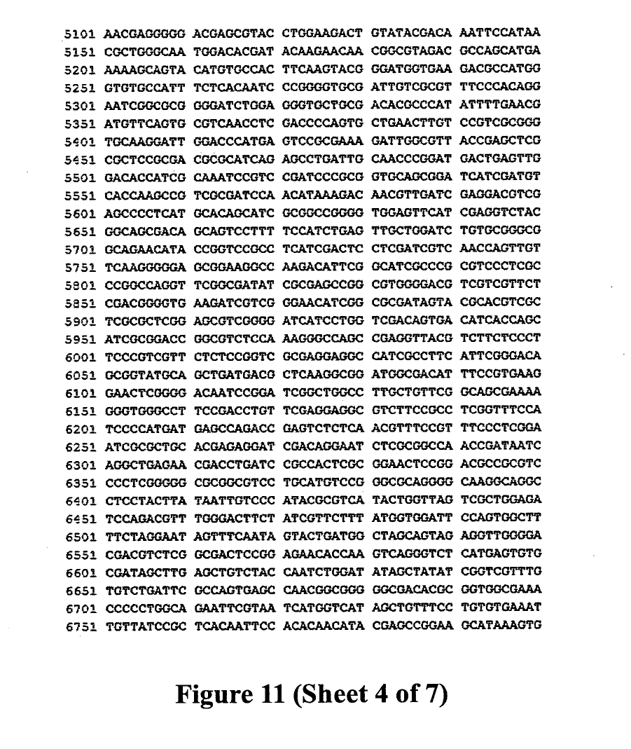

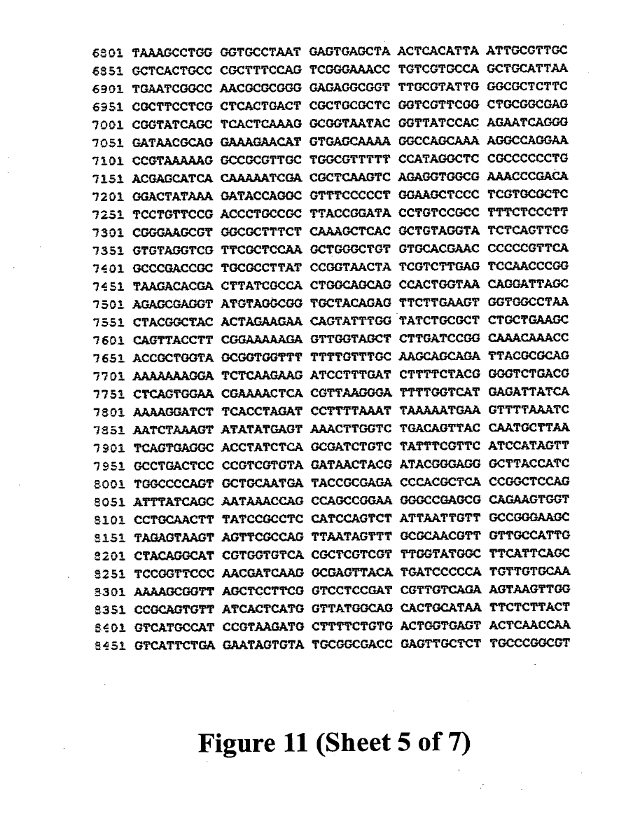

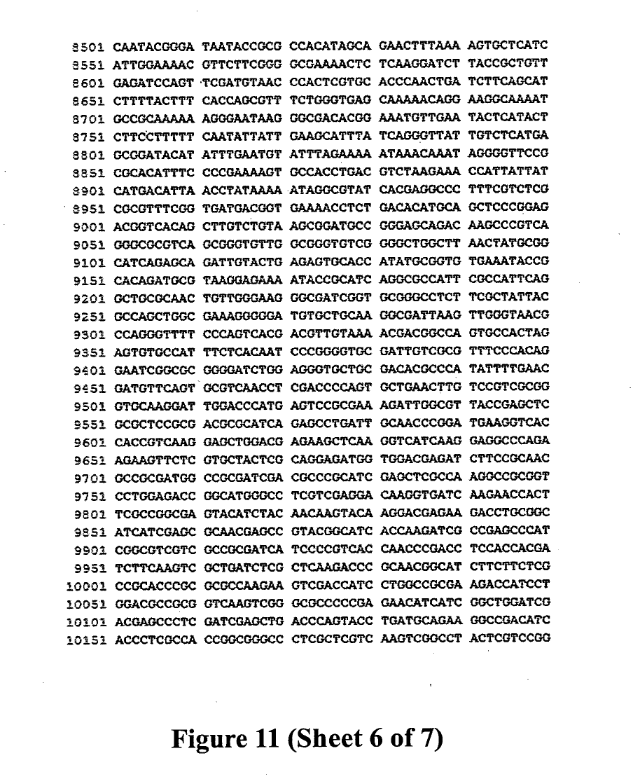

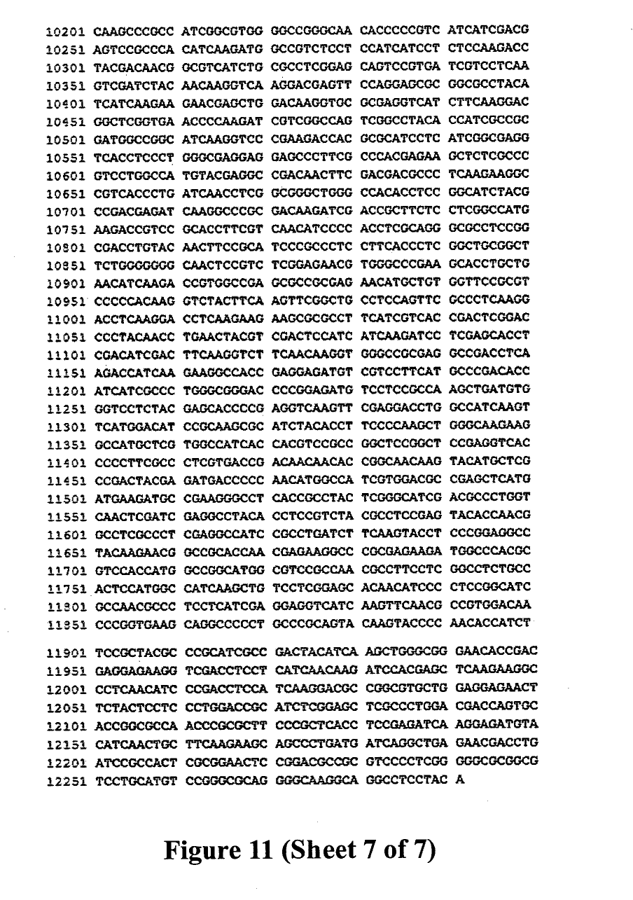

pBK1T2. Expression plasmid pBK1T2 is constructed by inserting into pBK1T a gene coding for the bifunctional aldehyde/alcohol dehydrogenase of Clostridium acetobutylicum (ATCC No. 824) (Uniprot Accession No. P33744), controlled by the promoter and terminator regions of the gene coding for the Methyl-malonyl CoA transcarboxilase (E.C. 2.1.3.1) of P. acidipropionici. Due to differences in the G+C content and codon usage between P. acidipropionici and C. acetobutylicum, said gene was designed by reverse translation of the primary amino acid sequence. For this, a codon table is generated from host ribosomal protein genes, which are highly expressed. The codons are selected to resemble this table and the overall host G+C content, avoiding recognition sites of host restriction enzymes. Inverted repeats were also avoided to disrupt mRNA secondary structures. Finally, adaptors for digestion with the restriction enzymes XbaI and HindIII are added to the 5′ and 3′ ends of this sequence, respectively. The sequence of this synthetic construct is provided in FIG. 6. The designed 2959 bp construct, containing the gene, its controlling regions and cloning adaptors is synthesized by Epoch Life Science (http://epochlifescience.com/Service/Gene Synthesis.aspx). The construct is then digested and cloned into the XbaI and HindIII sites of pBK1T to generate the expression shuttle plasmid pBK1T2. A schematic view of this plasmid is provided in FIG. 6 and its sequence in FIG. 11 (SEQ ID NO.: 154).

### Transformation:

pBK1T1 and pBK1T2 plasmids are first multiplied in E. coli GM2929 (dam-, dcm-) and are then recovered with high yield using standard procedures. Afterwards, these plasmids are transformed into electrocompetent cells of Propionibacterium freudenreichii (ATCC No. 6207) according to Kiatpapan and Murooka, 2001 (Appl. Microbiol. Biotechnol. 56:144-149) in order to obtain the appropriate methylation pattern to avoid digestion in the final host P. acidipropionici. Finally, the plasmids are recovered from P. freudenreichii and used to transform electrocompetent cells of P. acidipropionici (ATCC No. 4875). Transformants containing the expression plasmid pBK1T1 or pBK1T2 are selected in media containing 50 μg/mL thiostrepton and allowed to grow for 4-7 days.

### Growth:

Recovered colonies of P. acidipropionici containing the expression plasmid pBK1T1 or pBKT2 are used to inoculate Erlenmayer flasks containing 125 mL of culture media (0.5% yeast extract, 0.5% peptone, 0.1% KH₂PO₄, 0.2% (NH₄)₂HPO₄, 0.1% of saline solutions 1 and 2—solution 1:1% MgSO₄.7H₂O and 0.25% MnSO₄.H₂O; solution 2: 1% CaCl₂.2H₂O and 1% de CoCl₂.6H₂O; pH 6.8) with 50 μg/mL thiostrepton and 5% glycerol as a reduced carbon source. The culture is grown in anaerobiosis until reaching OD₆₀₀˜2.5 and is used to seed a bioreactor culture using the same media, as explained in comparative Example 1. The production of n-propanol from this reduced carbon source is measured by High-Performance Liquid Chromatography, coupled to a Refraction Index detector (Varian Chromatographer using a Aminex HPX-87H Organic Acid Column from Transgenomic, operating at room temperature and using 0.005 M H₂SO₄ as the eluent at a flux of 1 mL/min) and is compared to the production of this metabolite by a native P. acidipropionici strain (ATCC No. 4875). Native strains of P. acidipropionici are known to produce n-propanol from glycerol with a yield of approximately 4% (Barbirato et al., 1997, Appl. Microbiol. Biotechnol. 47: 441-446). Therefore, an increase in the production of this metabolite from glycerol can be attributed to the effect of the expression of the heterologous aldehyde/alcohol dehydrogenase gene.

### Example 3

### Fermentation of Sucrose by Propionibacterium Acidipropionici Using a Bioelectrical Reactor and a Mediator Molecule

A native strain of Propionibacterium acidipropionici (ATCC No. 4875) was used to study n-propanol production using sucrose as a carbon source. The bioelectrical reactor and different concentrations of mediator (cobalt sepulchrate) were utilized to drive the redox balance in order to obtain n-propanol.

P. acidipropionici was grown in a synthetic medium containing (per liter): 1 g KH2PO4, 2 g (NH4)2HPO4, 5 mg FeSO4.7H2O, 10 mg MgSO4.7H2O, 2.5 mg MnSO4.H2O, 10 mg CaCl2.6H2O, 10 mg CoCl2.6H2O, 10 g yeast extract (Oxoid), and the 9 g sucrose as a carbon source. The medium was autoclaved at 121° C. and 15 psig for 20 min. The cobalt sepulchrate (mediator) was added separately to the autoclaved media in order to avoid thermal molecular instability.

Batch fermentation in a bioelectrical reactor was performed in a 2.0 L fermentor APPLIKON containing 700 ml of culture medium. The temperature was set at 30° C. and the pH was maintained at 6.5 by automatic addition of 4 M NaOH, with 50 rpm agitation. Anaerobiosis was maintained by nitrogen sparing through the culture medium before fermentation began and after each sampling. The redox potential system consists of a working electrode (WE) (a graphite bar, area 4.9 cm² or 10.5 cm² and thickness of 3.0 mm) and a counter anode (a graphite bar, area 30 cm² and thickness of 3.0 mm in the counter electrode compartment filled with 40 ml 3 M KCl). The working electrode (WE) was poised at 150 mV more negative than the redox potential of the mediator (around −350 mV) using a DC voltage source (2.3-3.1 Volts). The current between working electrode and counter electrode was recorded using a computer interface. In order to define the correct voltage to be applied into the system, a cyclic voltametry experiment was performed using a potentiostat (PGSTAT 302N model from AUTOLAB) connected to the system. The bioreactor was inoculated with 70 ml of cells in exponential phase (OD˜3 to 5), which were grown in polypropylene test tubes at 30° C. Samples were collected every 2 hours. After measuring the optical density (OD₆₀₀), the remaining volume of the sample was centrifuged at 10,000 g for 6 min. The supernatant was stored at −20° C. until HPLC and SPME-GC/MS analysis.

Cell biomass was calculated by measuring the absorbance at 600 nm in a ULTROSPEC 2000 spectrophotometer UV/visible (Pharmacia Biotech) after appropriate dilution in water. For HPLC-R1 analysis, the samples were filtered through a 0.2 μm filter (Millipore). Propionic, succinic and acetic acids, n-propanol and sugars were separated and quantified by high-performance liquid chromatography (Waters 600 Chromatograph), using an ion exclusion column Aminex HPX-87H (Bio-Rad). Operating conditions were: 0.04 mol L⁻¹ H₂SO₄ degassed eluent, flow rate 0.4 mL min⁻¹, column temperature 35° C. and refractometer temperature 35° C.

The volatile products were confirmed by using the HS-SPME and gas chromatography mass spectrometry (GC-MS). The technique (SPME—Solid-phase microextraction) makes use of a fused silica optical fiber coated with a thin polymer layer to extract the analytes from a liquid (solution), from the headspace (HS) above a liquid or solid, or from a gaseous phase. All assays were carried out using 6 mL of fermented broth in pH 2-3 acidified in hydrochloric acid solution 3 mol L⁻¹. The experimental conditions of the assays were those indicated by the experimental design. Experimental conditions in SPME: Bath temperature (T: 30-35° C.), pre-equilibrium time (PET: 5 min), extraction time (Ext: 3 min) GC/MS analyses were obtained on an Agilent GC 6890/Hewlett-Packard 5973 gas chromatograph equipped with Stabilwax-DA capillary column (30 m×0.25 mm×0.25 μm) with helium (1 mL min⁻¹) as carrier gas. The oven temperature was programmed as follows: 40° C. for 3 min, then increased 5° C./min up to 130° C. e then increased 40° C./min to 210° C. The injection port was equipped with a 0.75 mm i d liner and the injector was maintained at 210° C. in the splitless mode. Under these conditions, no sample carry-over was observed on blank runs conducted between extractions. The volatile products were identified by comparing their experimental spectra with those of WILEY Mass Spectra Library and injection of standards.

Table 5 summarizes the final concentration of n-propanol obtained after several fermentations of varying mediator concentration and working cathode area, after 36 hrs of fermentation. In the control fermentation the voltage applied and mediator concentration were zero. As can be observed, n-propanol was detected in fermentations with mediator and their final concentration increase as a function of the mediator concentration, in the concentration range used, and working cathode area.

Using the native strain, n-propanol was formed with yields ranging from 1.0-9.6% depending on the conditions, with the best results corresponding to condition 0.8 mM cobalt sepulchrate (WE area 4.9 cm²). These results suggest that the native gene adh of P. acidipropionici is not efficient in the conversion of propionate to propanol. The next step consist of conducting fermentation with genetically modified strain expressing the gene from C. carboxidivorans as described in Example 2.

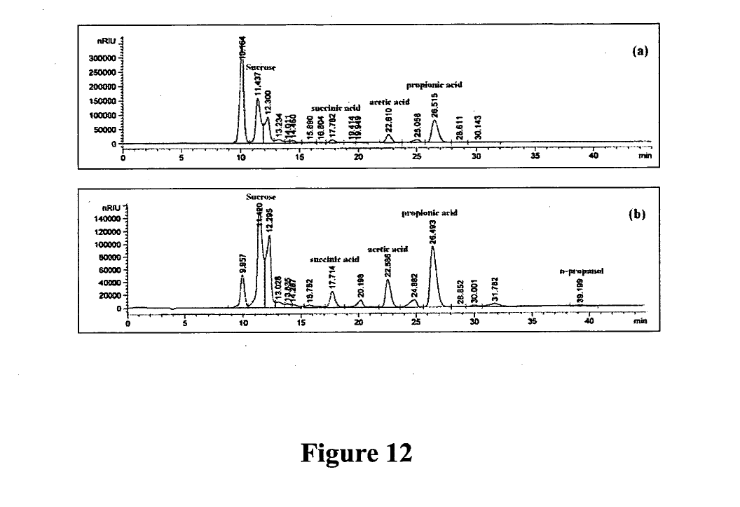

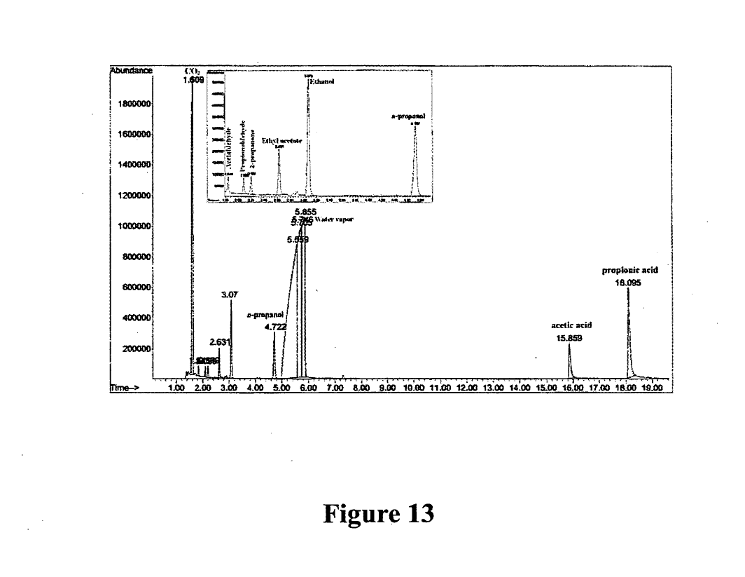

FIG. 12(a) and (b) shows HPLC and FIG. 13 shows GC-MS spectra after 36 hrs of control and 1.0 mM cobalt sepulchrate supplemented fermentations. The n-propanol peak appears only in the fermentation using bioelectrical reactor and the mediator molecule. FIG. 12 shows a GC-MS chromatogram obtained in the fermentation broth using 1.0 mM cobalt sepulchrate. The products propionic and acetic acids and n-propanol were confirmed by GC-MS in all fermentation experiments.

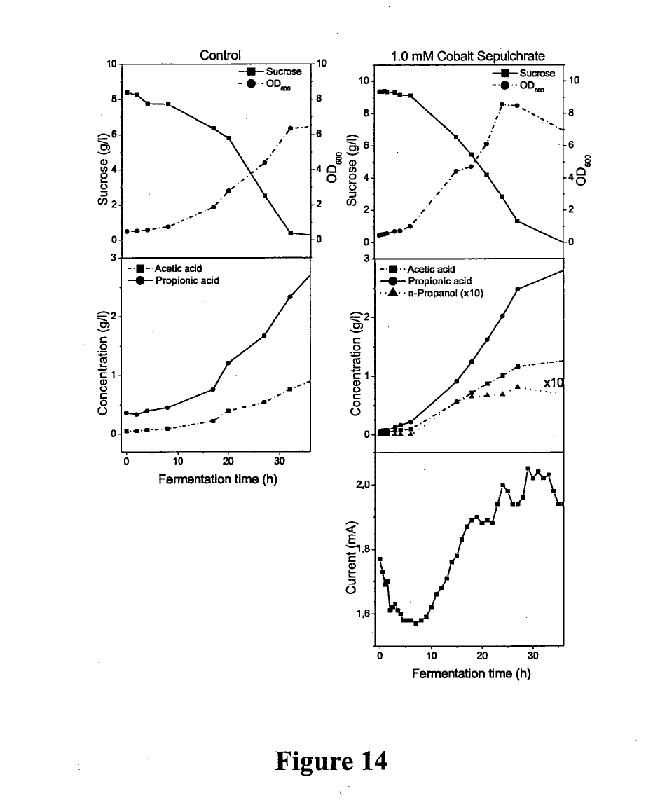

A time course for cell growth of the control and the 1.0 mM cobalt sepulchrate fermentation is shown in FIG. 14. In both fermentations it is possible to observe a similar behavior considering OD and formation of the common end-products, however in the fermentation using the mediator molecule n-propanol is produced at the beginning of the fermentation and its concentration increases following the cell growth.

TABLE 5
Final concentration of n-propanol obtained in five different
fermentations (duration of 36 hrs) by Propionibacterium acidipropionici
(ATCC No. 4875): control (no voltage applied and the mediator
concentration was zero), 0.5 (WE area 4.9 cm²), 0.8 (WE area 4.9 cm²),
1.0 (WE area 4.9 cm²), 0.8 (WE area 10.5 cm²), and
1.0 (WE area 10.5 cm²) mM mediator concentration.

| | n-Propanol |
|---|---|
| Fermentation | concentration (mg/L) |
| Control | ND |
| 0.5 mM Cobalt Sepulchrate | 25 |
| (WE area 4.9 cm²) | |
| 0.8 mM Cobalt Sepulchrate | 65 |
| (WE area 4.9 cm²) | |
| 1.0 mM Cobalt Sepulchrate | 81 |
| (WE area 4.9 cm²) | |
| 0.8 mM Cobalt Sepulchrate | 97 |
| (WE area 10.5 cm²) | |
| 1.0 mM Cobalt Sepulchrate | 180 |
| (WE area 10.5 cm²) | |

ND: Not detected

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for producing n-propanol comprising:
(a) providing a suitable carbon source for fermentation by a microorganism expressing the dicarboxylic acid pathway, reducing equivalents, and at least one gene coding for an enzyme that catalyzes the conversion of propionate/propionyl-CoA into n-propanol;
(b) contacting the carbon source and reducing equivalents with the microorganism under conditions favorable for the production of n-propanol by the microorganism; whereby a fermentation broth is produced; and
(c) recovering n-propanol from the fermentation broth.

2. The method of claim 1, wherein the microorganism has been genetically engineered to express one or more enzymes, whereby the microorganism is capable of converting propionate/propionyl-CoA to n-propanol.

3. The method of claim 2, wherein the microorganism is selected from the group consisting of: Propionigenium spp., Propionispira arboris, Propionibacterium spp., and Selenomonas.

4. The method of claim 2, wherein the enzyme is selected from the group consisting of:
aldehyde dehydrogenases that are capable of using propionic acid as a substrate;
aldehyde dehydrogenases that are capable of using an acyl-CoA intermediate as a substrate;
alcohol dehydrogenases that catalyze the conversion of an aldehyde to its corresponding primary alcohol; and
multifunctional enzymes that possess both aldehyde/alcohol dehydrogenase domains.

5. The method of claim 4, wherein the enzyme has alcohol dehydrogenase protein domain with e-value threshold below 1e-2.

6. The method of claim 4, wherein the enzyme has aldehyde dehydrogenase protein domain with e-value threshold below 1e-2.

7. The method of claim 4, wherein the aldehyde dehydrogenases are capable of using propionic acid as a substrate are selected from the group consisting of: Mus musculus (GenBank Accession No. AC162458.4) (SEQ ID NO.: 94); Clostridium botulinum A str. American Type Culture Collection (ATCC) No. 3502 (GenBank Accession No. AM412317.1) (SEQ ID NO.: 96); and Saccharomyces cerevisiae (GenBank Accession No. EU255273.1) (SEQ ID NO.: 98).

8. The method of claim 4, wherein the aldehyde dehydrogenases that are capable of using acyl-CoA intermediate as a substrate are selected from the group consisting of: Rhodococcus opacus (GenBank Accession No. AP011115.1) (SEQ ID NO.: 100); Entamoeba dispar (GenBank Accession No. DS548207.1) (SEQ ID NO.: 102); and Lactobacillus reuteri (GenBank Accession No. ACHG01000187.1) (SEQ ID NO.: 116).

9. The method of claim 4, wherein the alcohol dehydrogenases that catalyze the conversion of an aldehyde to its corresponding primary alcohol are selected from the group consisting of: Aspergillus niger (GenBank Accession No. AM270229.1) (SEQ ID NO.: 104); Streptococcus pneumoniae Taiwan19F-14 (GenBank Accession No. CP000921.1) (SEQ ID NO.: 106); and Salmonella enterica (GenBank Accession No. CP001127.1) (SEQ ID NO.: 108).

10. The method of claim 4, wherein the multifunctional enzymes that possess both aldehyde/alcohol dehydrogenase domains are selected from the group consisting of: Lactobacillus sakei (GenBank Accession No. CR936503.1) (SEQ ID NO.: 118); Giardia intestinalis (GenBank Accession No. U93353.1) (SEQ ID NO.: 120); Shewanella amazonensis (GenBank Accession No. CP000507.1) (SEQ ID NO.: 122); Thermosynechococcus elongatus (GenBank Accession No. BA000039.2) (SEQ ID NO.: 124); Clostridium acetobutylicum (GenBank Accession No. AE001438.3) (SEQ ID NO.: 126); and Clostridium carboxidivorans ATCC No. BAA-624T (GenBank Accession No. ACVMI000101.1) (SEQ ID NO.: 128).

11. The method of claim 1, wherein the fermentation broth further comprises ethanol and/or isopropanol.

12. The method of claim 11, wherein ethanol and/or isopropanol are recovered from fermentation broth.

13. The method of claim 1, wherein the microorganism has the expression of its gene encoding for an enzyme acetate kinase (E.C. 2.7.2.1) altered so as to diminish its activity.

14. The method of claim 1, wherein the reducing equivalents comprise NAD(P)H.

15. The method of claim 14, wherein the NAD(P)+ is reduced to NAD(P)H comprising the use of electrodes and a mediator molecule, an overpressure of H₂, or a microorganism expressing a NAD⁺-dependent formate dehydrogenase in the presence of formate.

16. The method of claim 14, further comprising contacting the fermentation broth with electrodes and a mediator molecule.

17. The method of claim 16, wherein mediator molecules are benzyl viologen, methyl viologen, anthraquinone 2,6-disulfonic acid, neutral red, cobalt sepulchrate, 1,4 dihydroxy-2-naphthoic acid (DHNA) and flavins.

18. The method of claim 16, wherein mediator molecules are compounds present in yeast extract and Propionibacterium spp. extract.

19. The method of claim 1, wherein the carbon source is sugarcane juice, sugarcane molasses, hydrolyzed starch, hydrolyzed ligno-cellulosic materials, glucose, sucrose, fructose, lactate, lactose, xylose or glycerol in any form or a mixture thereof.

20. A microorganism for using in the method as defined in claim 1.

21. A method for producing propylene comprising: dehydrating the n-propanol produced by the method as defined in claim 1 to produce propylene.

22. A method for producing propylene comprising: dehydrating in the same reactor n-propanol and isopropanol and/or ethanol produced by the method as defined in claim 1 to produce propylene.

23. A method for producing polypropylene comprising: polymerizing the propylene produced by the method as defined in claim 21 to produce polypropylene.


SEQUENCE LISTING (155 sequences)

SEQ ID NO: 1 - DNA, 366 bp, Propionibacterium acidi-propionici
atgaagctcaaggtgactgtcaacggcgtcgcttatgacgtcgacgttgacgttgacaagaccaacaattccccgatcccgccgatcatcttcggcggcgggtccggcggcccggctcgcgctgcgggcgggtccggcggtggcaaggccggtgcgggcgagattcccgccccgctggccggtaccgtcgccaagatcctcgtgaaggagggggaccaggtgaaggccggcgatgtcgtgctgactctcgaggccatgaagatggagaccgagatcaccgcgacctccgacgggaccgtcaagagcatcctggtcgccgtcggcgacgccgtccagggtggccagggcctggtggccgtcggctga

SEQ ID NO: 2 - protein, 121 aa, Propionibacterium acidi-propionici
MKLKVTVNGVAYDVDVDVDKTNNSPIPPIIFGGGSGGPARAAGGSGGGKAGAGEIPAPLAGTVAKILVKEGDQVKAGDVVLTLEAMKMETEITATSDGTVKSILVAVGDAVQGGQGLVAVG

SEQ ID NO: 3 - DNA, 1494 bp, Propionibacterium acidi-propionici
atgagtccgcgaaagattggcgttaccgagctcgcgctccgcgacgcgcatcagagcctgattgcaacccggatgtccatggaggacatggtcgatgcctgtgccgacatcgatgcggctggctactggtccgctgagtgctggggtggagctaccttcgattcctgtatccggttcctcaacgaggatccctgggagaggctgcggaccttccgcaagctgatgcccaacacccgcctgcagatgctgctgcgcggccagaacctcctcggctaccgccactacggtgacgacgtcgtcgacaagttcgtcgagaagtcggccgagaacggcatggacgtcttccgtgtcttcgacgccctcaacgatccccgcaacctcgagcgcgcgatggccgccgtcaagaagaccggcaagcacgcccagggcacgatctgctacaccacctccccgatccacaccccggagagcttcgtcaagcaggccgaccgcctcatcgacatgggtgccgactccatcgccttcaaggacatggccgccctgctgaagccgcagcccgcctatgacatcatcaagggcatcaaggagaaccaccccgacgtgcagatcaacctgcactgccactccaccaccggcgtcaccctggtgaccctcatgaaggccatcgaggccggcgtcgacgtcgtcgacaccgccatctcctcgatgtcgctggggccgggccacaacccgaccgagtccctggtcgagatgctcgagggcaccggctacgagaccggcctggacatggatcgcctcatcaagatccgcgaccacttcaagaaggtccgcccgaagtacaagaagttcgagtcgaagaccctggtcaacaccaacatcttccagtcccagatccccggcggcatgctctccaacatggagtcccagctggccgcccagggcgcctcggaccgcaccgacgaggtcatgaaggaggtgccgcgcgtccgcaaggatgccggctacccgcccctggtcaccccgtcctcccagatcgtcggcacccaggccgtgttcaacgtcctgatgggcaacggctcctacaagaacctgaccgccgagttcgccgacctgatgctcggctactacggcaagccggtcggcgagctcaaccccgagctcatcaagatggccgagaagcagaccggcaagaagccgatcgacgtgcgcccggccgatctcatcgataacgagtgggacgacctggtcaagcagtccgccgagctcgagggcttcgacggatccgacgaggacgtgctcaccaacgccctgttcccgggagtcgccccgaagttcttcaaggagcgcccgcagggccccaagagcgtcgcgatgaccgaggctcagatgaaggccgaggccgagggcaacggatcgacgtccgtctccggtccggtcaattacagcgtcacggtcggtggccgcagccacgacgtgaccgtcgagcctgcgtga

SEQ ID NO: 4 - protein, 497 aa, Propionibacterium acidi-propionici
MSPRKIGVTELALRDAHQSLIATRMSMEDMVDACADIDAAGYWSAECWGGATFDSCIRFLNEDPWERLRTFRKLMPNTRLQMLLRGQNLLGYRHYGDDVVDKFVEKSAENGMDVFRVFDALNDPRNLERAMAAVKKTGKHAQGTICYTTSPIHTPESFVKQADRLIDMGADSIAFKDMAALLKPQPAYDIIKGIKENHPDVQINLHCHSTTGVTLVTLMKAIEAGVDVVDTAISSMSLGPGHNPTESLVEMLEGTGYETGLDMDRLIKIRDHFKKVRPKYKKFESKTLVNTNIFQSQIPGGMLSNMESQLAAQGASDRTDEVMKEVPRVRKDAGYPPLVTPSSQIVGTQAVFNVLMGNGSYKNLTAEFADLMLGYYGKPVGELNPELIKMAEKQTGKKPIDVRPADLIDNEWDDLVKQSAELEGFDGSDEDVLTNALFPGVAPKFFKERPQGPKSVAMTEAQMKAEAEGNGSTSVSGPVNYSVTVGGRSHDVTVEPA

SEQ ID NO: 5 - DNA, 1575 bp, Propionibacterium acidi-propionici
atggctaacaagaagcgcgtcaagctcgccgagacgatggaaggtcgtctcgagcagctgaccgaacagcgtcacaagattgagctcggaggtggcgagaagcgcctccagaagcagcgcgacaagggcaagcagaccgcccgcgagcggatcgacaacctcctcgacgagtactccttcgacgaggtcggcgccttccgtgagcaccgcaccagcctcttcgggatggacaccgccgaggtgcccgccgacggcgtggtgaccggccgcggaaccgtcaacggccgcccggtccacgtggcctcccaggacttctccgtcatgggcgggtcggccggcgagacccagtcgaccaaggtcgtcgagaccatggagcaggcgctgctgaccggcacaccgttcctcttcttctacgattcgggcggcgctcggatccaggagggcatcgactcgctgtccggctacggcaagatgttctacgcgaacgtcaagctgtcgggtgtcgtgccgcagatcgccatcatcgccggcccctgcgccggtggtgcgtcctactccccggccctgaccgacttcatcatcatgacgaagaaggcccagatgttcatcacgggccccggcgtcatcaagtcggtgaccggcgaggacgtgaccggtgacgagctgggtggtgccgaggcgcacatgtcgacctccggcaacatccacttcgtggccgaggacgacgacgccgcggtgctgatcgcgcagaagctgctgagcttcctgccgcagaacaacacccaggacgcgacgatcgagttcccgaacaacgacatctccccgatccccgaactgcgcgacatcgtgccgatcgacgggaagaagggatatgacgtccgcgacgtcatctccaagatcgtcgactggggcgactacctggaggtcaaggcgggctgggcgaccaacatcgtcaccgccttcgccaggatcaacggtcgttcggtgggcatcgtggcgaaccagcccaaggtgatgtcgggctgcctcgacatcaacgcctcggacaaggccgcggagttcatcaacttctgcgactcgttcaacatcccgctggtgcagctggtggacgtccccggattccttccgggcgtccagcaggagtacggcggcatcatccgccatggcgcgaagatgctgtacgcctactccgaggccaccgtcccgaagatcaccgtggtgctgcgcaaggcctacggcggctcctacctggccatgtgcaaccgtgacctgggtgccgacgccgtctacgcctggcccaccgccgagatcgccgtgatgggcgccgagggcgcagccaatgtgatcttccgtcgtgagatcaaggcctccgatgatcccgccgccacccgcgcggagaagatcgaggagtaccagacggcgttcaacacgccgtatgtggcggctgcccgcggacaggtcgatgacgtcatcgaccccgccgacacccgtcgcaggatcactgccgctctggagacctacgccacgaagcgtcagtcccgtccggccaagaagcacggcaatatgccgtgctga

SEQ ID NO: 6 - protein, 524 aa, Propionibacterium acidi-propionici
MANKKRVKLAETMEGRLEQLTEQRHKIELGGGEKRLQKQRDKGKQTARERIDNLLDEYSFDEVGAFREHRTSLFGMDTAEVPADGVVTGRGTVNGRPVHVASQDFSVMGGSAGETQSTKVVETMEQALLTGTPFLFFYDSGGARIQEGIDSLSGYGKMFYANVKLSGVVPQIAIIAGPCAGGASYSPALTDFIIMTKKAQMFITGPGVIKSVTGEDVTGDELGGAEAHMSTSGNIHFVAEDDDAAVLIAQKLLSFLPQNNTQDATIEFPNNDISPIPELRDIVPIDGKKGYDVRDVISKIVDWGDYLEVKAGWATNIVTAFARINGRSVGIVANQPKVMSGCLDINASDKAAEFINFCDSFNIPLVQLVDVPGFLPGVQQEYGGIIRHGAKMLYAYSEATVPKITVVLRKAYGGSYLAMCNRDLGADAVYAWPTAEIAVMGAEGAANVIFRREIKASDDPAATRAEKIEEYQTAFNTPYVAAARGQVDDVIDPADTRRRITAALETYATKRQSRPAKKHGNMPC

SEQ ID NO: 7 - DNA, 372 bp, Propionibacterium freudenreichii shermanii
atgaaactgaaggtaacagtcaacggcactgcgtatgacgttgacgttgacgtcgacaagtcacacgaaaacccgatgggcaccatcctgttcggcggcggcaccggcggcgcgccggcaccgcgcgcagcaggtggcgcaggcgccggtaaggccggagagggcgagattcccgctccgctggccggcaccgtctccaagatcctcgtgaaggagggtgacacggtcaaggctggtcagaccgtgctcgttctcgaggccatgaagatggagaccgagatcaacgctcccaccgacggcaaggtcgagaaggtccttgtcaaggagcgtgacgccgtgcagggcggtcagggtctcatcaagatcggctga

SEQ ID NO: 8 - protein, 123 aa, Propionibacterium freudenreichii shermanii
MKLKVTVNGTAYDVDVDVDKSHENPMGTILFGGGTGGAPAPRAAGGAGAGKAGEGEIPAPLAGTVSKILVKEGDTVKAGQTVLVLEAMKMETEINAPTDGKVEKVLVKERDAVQGGQGLIKIG

SEQ ID NO: 9 - DNA, 1518 bp, Propionibacterium freudenreichii shermanii
atgagtccgcgagaaattgaggtttccgagccgcgcgaggttggtatcaccgagctcgtgctgcgcgatgcccatcagagcctgatggccacacgaatggcaatggaagacatggtcggcgcctgtgcagacattgatgctgccgggtactggtcagtggagtgttggggtggtgccacgtatgactcgtgtatccgcttcctcaacgaggatccttgggagcgtctgcgcacgttccgcaagctgatgcccaacagccgtctccagatgctgctgcgtggccagaacctgctgggttaccgccactacaacgacgaggtcgtcgatcgcttcgtcgacaagtccgctgagaacggcatggacgtgttccgtgtcttcgacgccatgaatgatccccgcaacatggcgcacgccatggctgccgtcaagaaggccggcaagcacgcgcagggcaccatttgctacacgatcagcccggtccacaccgttgagggctatgtcaagcttgctggtcagctgcttgacatgggtgctgattccatcgccctgaaggacatggccgccctgctcaagccgcagccggcctacgacatcatcaaggccatcaaggacacttacggccagaagacgcagatcaacctgcactgccactccaccacgggtgtcaccgaggtctccctcatgaaggccatcgaggccggcgtcgacgtcgtcgacaccgccatctcgtccatgtcgctcggcccgggccacaaccccaccgagtcggtcgccgagatgctcgagggcaccgggtacaccaccaaccttgactacgatcgcctgcacaagatccgcgatcacttcaaggccatccgcccgaagtacaagaagttcgagtcgaagacgcttgtcgacacctcgatcttcaagtcgcagatccccggcggcatgctctccaacatggagtcgcagctgcgcgcccagggcgccgaggacaagatggacgaggtcatggccgaggttccgcgcgtccgcaaggccgccggcttcccgcccctggtcaccccgtccagccagatcgtcggcacgcaggccgtgttcaacgtgatgatgggcgagtacaagaggatgaccggcgagttcgccgacatcatgctcggctactacggcgccagcccggccgatcgcgatccgaaggtggtcaagttggccgaggagcagtccggcaagaagccgatcacccagcgcccggccgatctgctgccccccgagtgggagaagcagtccaaggaggccgcgacgctcaagggcttcaacggcaccgacgaggacgtgctcacctatgcactgttcccgcaggtcgctccggtcttcttcgagcatcgcgccgagggcccgcacagcgtggctctcaccgatgcccagctgaaggccgaggccgagggcgacgagaagtcgctcgccgtggccggtcccgtcacctacaacgtgaacgtgggcggaaccgtccgcgaagtcaccgttcagcaggcgtga

SEQ ID NO: 10 - protein, 505 aa, Propionibacterium freudenreichii shermanii
MSPREIEVSEPREVGITELVLRDAHQSLMATRMAMEDMVGACADIDAAGYWSVECWGGATYDSCIRFLNEDPWERLRTFRKLMPNSRLQMLLRGQNLLGYRHYNDEVVDRFVDKSAENGMDVFRVFDAMNDPRNMAHAMAAVKKAGKHAQGTICYTISPVHTVEGYVKLAGQLLDMGADSIALKDMAALLKPQPAYDIIKAIKDTYGQKTQINLHCHSTTGVTEVSLMKAIEAGVDVVDTAISSMSLGPGHNPTESVAEMLEGTGYTTNLDYDRLHKIRDHFKAIRPKYKKFESKTLVDTSIFKSQIPGGMLSNMESQLRAQGAEDKMDEVMAEVPRVRKAAGFPPLVTPSSQIVGTQAVFNVMMGEYKRMTGEFADIMLGYYGASPADRDPKVVKLAEEQSGKKPITQRPADLLPPEWEKQSKEAATLKGFNGTDEDVLTYALFPQVAPVFFEHRAEGPHSVALTDAQLKAEAEGDEKSLAVAGPVTYNVNVGGTVREVTVQQA

SEQ ID NO: 11 - DNA, 1818 bp, Propionibacterium freudenreichii shermanii
atggctgaaaacaacaatttgaagctcgccagcaccatggaaggtcgcgtggagcagctcgcagagcagcgccaggtgatcgaagccggtggcggcgaacgtctcgtcgagaagcaacattcccagggtaagcagaccgctcgtgagcgcctgaacaacctgctcgatccccattcgttcgacgaggtcggcgctttccgcaagcaccgcaccacgttgttcggcatggacaaggccgtcgtcccggcagatggcgtggtcaccggccgtggcaccatccttggtcgtcccgtgcacgccgcgtcccaggacttcacggtcatgggtggttcggcttggcgagacgcagtccacgaaggtcgtcgagacgatggaacagcgctgctcaccggcacgcccttcctgttcttctacgattcgggcggccggatccaggagggcatcgactcgctgagcggttacggcaagatgttcttcgccaacgtgaagctgtcgggcgtcgtgccgcagatcgccatcattgccggcccctgtgcgtgcgcctcgtattcgccggcactgactgacttcatcatcatgaccaagaaggcccatatgttcatcacgggcccccaggtcatcaagtcggtcaccggcgaggatgtcaccgctgacgaactcggtggcgctgagcccatatggccatctcgggcaatatacttcgtggccgaggacgacgacgccgcggagctcattgccaagaagctgctgagcttccttccgcagaacaacactgaggaagcatccttcgtcaacccgaacaatgacgtcagccccaataccgagctgcgcgacatcgttccgattgacggcaagaagggctatgacgtgcgcgatgtcattgccaagatcgtcgactggggtgactacctcgaggtcaaggccggctatgccaccaacctcgtgaccgccttcgcccgggtcaatggtcgttcggtgggcatcgtggccaatcagccttcggtgatgtcgggttgcctcgacatcaacgcctctgacaaggccgccgaattcgtgaatttctgcgattcgttcaacatcccgctggtgcagctggtcgacgtgccgggcttcctgcccgtgcagcaggagtacggcggcatcattcgccatgggcgcaagatgctgtacgcctactccgaggccaccgtgccgaagatcacgtgtctcgcaacgcctacggcggctcctacctggccatgtgcaaccgtgaccttggtgccgacgccgtgtacgcctgtgcccagcgccgagattgcggtgatgggcgccgagggtgcggcaaatgtgatcttccgcaaggagatcaaggctgccgacgatcccgacgccatgcgcgccgagaagatcgaggagtaccagaacggttcaacacgccgtacgtggcgcgcccgcggtcaggtcgacgacgtgattgacccggctgatacccgtcgaaagattgcttccgccctggagatgtacgccaccaagcgtcagacccgcccggcgaagaagccatggaaacttcccctgctgagcgaggaggaaattatggctgatgaggaagagaaggacctgatgatcgccacgctcaacaagcgcgtcgcgtcattggagtctgagttgggttcactccagagcgatacccagggtgtcaccgaggacgtactgacggccatttcggccgttgcggcctatctcggcaacgatggatcggctgaggtcgtccatttcgccccgagcccgaactgggtccgcgagggtcgtcgggctctgcagaaccattccattcgttga

SEQ ID NO: 12 - protein, 605 aa, Propionibacterium freudenreichii shermanii
MAENNNLKLASTMEGRVEQLAEQRQVIEAGGGERLVEKQHSQGKQTARERLNNLLDPHSFDEVGAFRKHRTTLFGMDKAVVPADGVVTGRGTILGRPVHAASQDFTVMGGSAWRDAVHEGRRDDGTALLTGTPFLFFYDSGGRIQEGIDSLSGYGKMFFANVKLSGVVPQIAIIAGPCACASYSPALTDFIIMTKKAHMFITGPQVIKSVTGEDVTADELGGAEPIWPSRAIYFVAEDDDAAELIAKKLLSFLPQNNTEEASFVNPNNDVSPNTELRDIVPIDGKKGYDVRDVIAKIVDWGDYLEVKAGYATNLVTAFARVNGRSVGIVANQPSVMSGCLDINASDKAAEFVNFCDSFNIPLVQLVDVPGFLPVQQEYGGIIRHGRKMLYAYSEATVPKITCLATPTAAPTWPCATVTLVPTPCTPVPSAEIAVMGAEGAANVIFRKEIKAADDPDAMRAEKIEEYQNGSTRRTWRARGQVDDVIDPADTRRKIASALEMYATKRQTRPAKKPWKLPLLSEEEIMADEEEKDLMIATLNKRVASLESELGSLQSDTQGVTEDVLTAISAVAAYLGNDGSAEVVHFAPSPNWVREGRRALQNHSIR

SEQ ID NO: 13 - DNA, 363 bp, Propionibacterium acnes
atgaagctcaaggtgaccgtcaatgacgtcgcatacgacgttgacgttgacgttgataagaccgccaatgcgccgatggcgccgatcctctttggtggcggtgccggcggcccgatgaaggcatccggtggcggcgccggtaaggccggtgagggcgaggttcccgcaccgctagctgggactgttgccaagatcctggtggccgaaggagatgccgtcaaggccggtcaggtgctcctgaccctcgaggccatgaagatggagaccgagatcaatgccccggcggacggaaccgtcaaggggatcctggtggctgtcggtgacgccgtccagggtggtcagggcctggtggctctgggctga

SEQ ID NO: 14 - protein, 120 aa, Propionibacterium acnes
MKLKVTVNDVAYDVDVDVDKTANAPMAPILFGGGAGGPMKASGGGAGKAGEGEVPAPLAGTVAKILVAEGDAVKAGQVLLTLEAMKMETEINAPADGTVKGILVAVGDAVQGGQGLVALG

SEQ ID NO: 15 - DNA, 1494 bp, Propionibacterium acnes
atgagtccacgaaagattggcgttaccgagctcgtgctccgcgacgcgcatcagagcctgcttgccactcgcatggccatggaggacatggttgatgcctgtgccgacattgatgcggcaggcttctggtccgttgaatgctggggcggagctaccttcgattcttgcatccgattcctcaacgaagacccatgggagcgtctgcgtactttccgcaagctgctgccgaactcccggttgcagatgctgctgcgtggccaaaaccttctgggctaccgccactacaacgacgaggtcgtcgacaagtttgtcgagaagtcggccgagaacggcatggacgtgttccgggtgttcgacgctctgaacgatcctcgcaaccttgagcacgcgatggcagccgtcaagaagaccggcaagcacgcccagggcaccatctgctacaccacttccccgattcacaccccagagagcttcgtcaagcaggccgatcgtctcatcgacatgggtgccgactcgatcgccttcaaggacatggctgctttgctcaagccgcagcctgcctacgacatcatcaagggcattaaggagaaccatccggacgtgcagatcaacctgcactgccactccaccacgggcgtcaccctggtcaccctgcagaaggccatcgaggctggtgtcgacgtcgtcggcaccgctatctcgtcgatgtcgctcggcccggggcacaacccaaccgagtctttggtcgagatgctcgagggcaccgagtacatcaccggcctcgacatggatcgcctgctcaagatccgcgaccacttcaagaaggtgcgtccgaagtacaagaagttcgagtcgaagacgctggtcaacaccaacatcttccagtcccagatcccgggcggaatgctctccaacatggagtcccagctcgaggcccagggtgctggagaccgcatggatgaggtcatgaaggaggtgccgcgcgttcgtaaggatgccggctacccgccgctggtcaccccgtcctcccagatcgtgggaacccaggcggtgttcaacgtcctgatgggcaatggttcgtacaagaacctcactgccgagtttgccgacctcatgcttggctactacggcaagcccattggcgagctcaatcccgagatcgttgagatggccaagaagcagaccggcaaggagccgatcgactgccgtcccgccgacctgctcgagcctgagtgggaccagctggtcgagcaggccaagagtcttgagggcttcgacggctccgacgaggacgttcttaccaacgccctgttcccgggagttgccccgaagttcctcaaggaacgcgcacagggcccgaagagcgtcgcgatgaccgaggcacagctgaaggccgagaaggaaggcaccggcgctgccggcatcgccggaccggtcaactacaacgtgacggtcggtggcaacagccaccaggtgaccgtcgagcctgcgtga

SEQ ID NO: 16 - protein, 497 aa, Propionibacterium acnes
MSPRKIGVTELVLRDAHQSLLATRMAMEDMVDACADIDAAGFWSVECWGGATFDSCIRFLNEDPWERLRTFRKLLPNSRLQMLLRGQNLLGYRHYNDEVVDKFVEKSAENGMDVFRVFDALNDPRNLEHAMAAVKKTGKHAQGTICYTTSPIHTPESFVKQADRLIDMGADSIAFKDMAALLKPQPAYDIIKGIKENHPDVQINLHCHSTTGVTLVTLQKAIEAGVDVVGTAISSMSLGPGHNPTESLVEMLEGTEYITGLDMDRLLKIRDHFKKVRPKYKKFESKTLVNTNIFQSQIPGGMLSNMESQLEAQGAGDRMDEVMKEVPRVRKDAGYPPLVTPSSQIVGTQAVFNVLMGNGSYKNLTAEFADLMLGYYGKPIGELNPEIVEMAKKQTGKEPIDCRPADLLEPEWDQLVEQAKSLEGFDGSDEDVLTNALFPGVAPKFLKERAQGPKSVAMTEAQLKAEKEGTGAAGIAGPVNYNVTVGGNSHQVTVEPA

SEQ ID NO: 17 - DNA, 1575 bp, Propionibacterium acnes
atggctgagaagaaaccaatcaagctggccgataccatggccggccggatcgagcagctcgccgacgagcgccacgctgtggagcttggcgggggcgaggatcgcctgcaaaagcagcgtgacaggggcaagcagacagcccgtgaacggatcgacaacctcgttgatgcttattccttcgatgaggtgggtgcgttccgtaagcaccgcaccacccttttcggcatggacaaggccgaagttcccgccgacggcgtagtcaccggtcgtgcgaccatccatggtcgcccggtccacatcgcgtctcaggacttcaccgtcatgggtgggtcggctggcgagacccagtcgacgaaggtcgtcgagacgatggaacagtccctgctgaccggcactccgtttctgttcttctatgactcgggcggcgcccgaattcaagaaggcatcgactcgctgtccgggtacggcaagatgttctacgcgaacgtcaagctgtcgggcgtcgtgccgcagatcgccatcattgctggcccctgcgccggcggcgcctcctattccccggccctgaccgacttcatcatcatgacgaagaaggcccacatgttcattacgggccccggagtcatcaagtcggttaccggtgaggaggtgactgctgacgacctgggtggtgcggatgcgcacatgtccacctcgggcaatatccacttcgtggccgaagatgacgacgccgcagtgctcatcgcgcagaagttgctgagcttcctgccgcaaaacaacactgaggacgcccagatctccaaccccaatgacgatgtctccccgcagcctgagctgcgcgacatcgttccgctggatggtaagaagggctacgacgtccgcgacgtcatctccaagatcgtcgactggggcgactacctagaggtcaaggccggttgggcgaccaacatcgtcaccgcctttgcccgggtcaatggtcgtaccgtcggcatcgtggccaaccagccgaaggtgatgtcgggttgccttgacatcaatgcttcggacaaggctgccgagttcattaccttctgcgactcgttcaatattccgttggtgcagttggttgacgttcctggcttcctgcctggtgtccagcaggagtacggcggcatcatccgccacggcgcgaagatgctgtatgcctactccgaggccaccgtcccgaagatcaccgtggtgctgcgtaaggcttacggcggctcctacctggccatgtgcaaccgtgacctgggtgctgacgccgtctatgcctggccgagcgcggagattgcggtgatgggtgccgatggcgctgccaacgtcattttccgtcgccagatcaaggactctgaggatcccgcagccacccgtgccgcgaagatcgaggagtaccgcaacgccttcaacacgccttacgtggctgccgcccgtggacaggttgacgacgtgatcgatcccgcggacacccgtcgcaagatcaccgccgctctggagacctacgccactaagcgtcagtcccgtccggccaagaagcacggcgtcatgccttgctga

SEQ ID NO: 18 - protein, 524 aa, Propionibacterium acnes
MAEKKPIKLADTMAGRIEQLADERHAVELGGGEDRLQKQRDRGKQTARERIDNLVDAYSFDEVGAFRKHRTTLFGMDKAEVPADGVVTGRATIHGRPVHIASQDFTVMGGSAGETQSTKVVETMEQSLLTGTPFLFFYDSGGARIQEGIDSLSGYGKMFYANVKLSGVVPQIAIIAGPCAGGASYSPALTDFIIMTKKAHMFITGPGVIKSVTGEEVTADDLGGADAHMSTSGNIHFVAEDDDAAVLIAQKLLSFLPQNNTEDAQISNPNDDVSPQPELRDIVPLDGKKGYDVRDVISKIVDWGDYLEVKAGWATNIVTAFARVNGRTVGIVANQPKVMSGCLDINASDKAAEFITFCDSFNIPLVQLVDVPGFLPGVQQEYGGIIRHGAKMLYAYSEATVPKITVVLRKAYGGSYLAMCNRDLGADAVYAWPSAEIAVMGADGAANVIFRRQIKDSEDPAATRAAKIEEYRNAFNTPYVAAARGQVDDVIDPADTRRKITAALETYATKRQSRPAKKHGVMPC

SEQ ID NO: 19 - DNA, 984 bp, Propionibacterium acidi-propionici
atgagcactgctcccgtcaagattgctgtgaccggcgccgccggtcagatctgttacagcctgttgttccgcatcgccagtggttcgctgctcggcagcacccccatcgagctgcgtctgctggagatcacccccgctctcaaggctctcgagggtgtcgtcatggagctcgatgacggtgccttcccgaacctcgtcaacatcgagatcggcgatgaccccaagaaggtcttcgacggcgtcaacgccgccttcctggtcggcgccatgccccgcaaggccggcatggagcgctccgatctgctgagcaagaacggcgcgatcttcaccgctcagggcaaggccctcaatgacgtcgccgccgacgacgtcaaggtcctggtgaccggcaacccggccaacaccaacgccctgatcgcggccaccaacgccgtggacatcccgaacgaccacttcgccgccctgacccgtctggaccacaaccgcgccaagacccagctggcccgcaaggtcggcgccggcgtggccgacgtcaagcacatgaccatctggggcaaccactcctccacccagtaccccgacgtcttccacgccgaggtcgcgggcaagagcgctgccgatctggtcgacgaggcctgggtcgagaacgagttcatcccgactgtcgccaagcgcggcgccgctatcatcgccgcccgcggttcctcttctgccgcctcggccgccaacgcgaccgtcgagtgcatgcacgactggcttggcagcacccccgagggcgactgggtctcgatggcagttccgtccgacggctcctacggggtgcccgagggcctcatctcgtccttcccggtcaccgtctccgacggcaaggtcgagatcgtccagggcctggacatcgactccttctcccgcggcaagatcgacgcctccgcagctgagctgcaggatgagcgcgacgccgtcaaggagctcggcctcatctga

SEQ ID NO: 20 - protein, 327 aa, Propionibacterium acidi-propionici
MSTAPVKIAVTGAAGQICYSLLFRIASGSLLGSTPIELRLLEITPALKALEGVVMELDDGAFPNLVNIEIGDDPKKVFDGVNAAFLVGAMPRKAGMERSDLLSKNGAIFTAQGKALNDVAADDVKVLVTGNPANTNALIAATNAVDIPNDHFAALTRLDHNRAKTQLARKVGAGVADVKHMTIWGNHSSTQYPDVFHAEVAGKSAADLVDEAWVENEFIPTVAKRGAAIIAARGSSSAASAANATVECMHDWLGSTPEGDWVSMAVPSDGSYGVPEGLISSFPVTVSDGKVEIVQGLDIDSFSRGKIDASAAELQDERDAVKELGLI

SEQ ID NO: 21 - DNA, 987 bp, Propionibacterium freudenreichii shermanii
gtgagcactacacccgtcaaggttgccgttaccggcgctgccggccagatttgttacagcctgctgttccgcattgcgtccggctcgctgctcggcgatacgcctatcgagcttcgcctgctcgagatcacccccgccctgccgcgcctcgaaggcgtggtcatggagctcgacgactgcgcattccccaatctcgccggcgtcgagatcggcgacgaccccgagaaggtcttcgacggagccaaccttgccatgctcgtcggcgccatgccccgcaaggagggcatggatcgctccgatctgcttggcgcgaacggcaagatcttcaccggtcagggcaaggccctcaacaaggttgcggccgacgatgtgcgcatcctggtcaccggcaacccggccaacaccaacgccctgatcgccaaggacaatgcccctgacattcccgacgatcgcttcagcgccctgacgcgcctggaccacaaccgcgccaagtcgatgctcgcgaagaagctgggcgtcaacgtgggcgaggtcaccaacatgaccatctggggcaaccactccaacacccagttccccgacctgttccacaccaaggtcggcggcaagaacgcctacgagctggtcaacgacgaagcctggtacgagaacacctacatccccgaggtcgccaagcgcggcggcgcggtgatcaaggcccgcggtgcgagctcggccgcctcggccgccaacgccaccgttgaggccatgcacgactgggccgtcggcaccccggccaacgactgggtctcgatgtcggtcgtctccgatggttcctacggcgtgccggagggcctcatcagctccttcccggtgacctgcaaggacggcaagtacgagatcgtccagggcctcgacctgaacgacttctccaagaagaagatcgctgccaccgtcgacgagctcaccaaggagcagggcgaggttcgcgagatgggcctcatctga

SEQ ID NO: 22 - protein, 328 aa, Propionibacterium freudenreichii shermanii
MSTTPVKVAVTGAAGQICYSLLFRIASGSLLGDTPIELRLLEITPALPRLEGVVMELDDCAFPNLAGVEIGDDPEKVFDGANLAMLVGAMPRKEGMDRSDLLGANGKIFTGQGKALNKVAADDVRILVTGNPANTNALIAKDNAPDIPDDRFSALTRLDHNRAKSMLAKKLGVNVGEVTNMTIWGNHSNTQFPDLFHTKVGGKNAYELVNDEAWYENTYIPEVAKRGGAVIKARGASSAASAANATVEAMHDWAVGTPANDWVSMSVVSDGSYGVPEGLISSFPVTCKDGKYEIVQGLDLNDFSKKKIAATVDELTKEQGEVREMGLI

SEQ ID NO: 23 - DNA, 984 bp, Propionibacterium acnes
atgacccagaccccagtcaagattgccgtcaccggagctgctggccagatttgctacagcttgttgttccgcattgcttccggttcgctgctcggagacactcctatcgagcttcgccttctcgagatcaccccggccctcaaagcccttgagggcgtcgtcatggagcttgacgactgcgccttcggcaatctcgtgaatatcgagatcggtgacgatccgaagaaggtctttgacggagttaacgccgccttcctcgtcggcgccatgccccgcaaggcgggtatggagcgttccgatctgctcaccaagaacggggctatcttcaccgcccaaggcaaggccctcaacgacgtcgccgccgacgacgtccgcgttctcgtcaccggtaaccccgctaacaccaacgccctcatcgctgctaccaacgcggtcgacatcccgaacaaccacttcgccgccttgactcgtctagatcacaaccgcgccaagacgcagcttgcccgcaagaccggaaagactgtcaatgacgtccgtcacatgaccatctggggcaaccactcctccacccagtaccccgacgtcttccacgccgaggtcgccggccagaaggcaaccaacctggtgaacgaagcttggatcgagaacgagttcatcccaaccgtcgccaaacgtggcgctgccatcatcgacgcgcgcggcgcctcctcggctgcctcggccgctaacgccaccgtcgagtgcatgcgcgactggatgggctccactcctgagggagactgggtgtccatggcgattccgtctgacggctcttacggcgtgcccgagggcctcatctcttccttcccggtgaccatcaccaacggcaaggtcgagattgttcagggtcttgacatcgacgacttctcgcgcgccaagatcgacgcctctgccaaggagctggccgacgaacgtgacgccgtcaaggaactcggcctcatctga

SEQ ID NO: 24 - protein, 327 aa, Propionibacterium acnes
MTQTPVKIAVTGAAGQICYSLLFRIASGSLLGDTPIELRLLEITPALKALEGVVMELDDCAFGNLVNIEIGDDPKKVFDGVNAAFLVGAMPRKAGMERSDLLTKNGAIFTAQGKALNDVAADDVRVLVTGNPANTNALIAATNAVDIPNNHFAALTRLDHNRAKTQLARKTGKTVNDVRHMTIWGNHSSTQYPDVFHAEVAGQKATNLVNEAWIENEFIPTVAKRGAAIIDARGASSAASAANATVECMRDWMGSTPEGDWVSMAIPSDGSYGVPEGLISSFPVTITNGKVEIVQGLDIDDFSRAKIDASAKELADERDAVKELGLI

SEQ ID NO: 25 - DNA, 969 bp, Selenomonas ruminantium lactilytica
atgaaggtaacagttgttggtgcaggtaacgttggcgctaccgtagcaaatgtgctggcagtgaagaatttctgcagcgaagttatgctcgtggacatcaagaagggctttgctgaaggcaaggctatggacatcatgcagacggcacatctgctgaactttgacaccacggtaacgggcgttacggctgaaatcggtgacgagaacggttatgcaccgacggaaggttccgatgttgtggttgttacttccggcatgccgcgtaaaccgggtatgactcgcgaagaactcatcggcgtaaatgcaaagattgtcaaaggtgttgttgaccaggcgctgaagtattctcccaacgctatcttcatcatcatctccaacccgatggacgctatgacgttcctgacgctgaaagattccaagctgccccgcaaccgcgttctcggtcagggcggtatgctcgacagcagccgtttccgttatttcctgtccaaggctctgcaggaagctggctatccggcaaccccgacggacatcgatggcacggttatcggcggtcacagcgacaagaccatggttcccctcacgagcctggctacctatcgcggcatccccgtttctcagctgctctccaaagagcagctggatgatgctgtagctcagacgaaggttggcggcgctacgctgacgggcctgctgggcacttccgcttggtacgctccgggcgcagcagctgctgccatggttgaggctatcgctctcgatgccaagaagctcatgccttgctgcgtatacctcgatggcgaatacggcgaaaaagatctctgcatcggcgtacccgttatcctgggcaagaacggtctggaaaagatcgtggaatacaagctcgaaggcgacgagaaggctaagtttgatgaaagcgtagctgctgcccgcaacacgaactccaaactcggcgatgctttgaaataa

SEQ ID NO: 26 - protein, 322 aa, Selenomonas ruminantium lactilytica
MKVTVVGAGNVGATVANVLAVKNFCSEVMLVDIKKGFAEGKAMDIMQTAHLLNFDTTVTGVTAEIGDENGYAPTEGSDVVVVTSGMPRKPGMTREELIGVNAKIVKGVVDQALKYSPNAIFIIISNPMDAMTFLTLKDSKLPRNRVLGQGGMLDSSRFRYFLSKALQEAGYPATPTDIDGTVIGGHSDKTMVPLTSLATYRGIPVSQLLSKEQLDDAVAQTKVGGATLTGLLGTSAWYAPGAAAAAMVEAIALDAKKLMPCCVYLDGEYGEKDLCIGVPVILGKNGLEKIVEYKLEGDEKAKFDESVAAARNTNSKLGDALK

SEQ ID NO: 27 - DNA, 1407 bp, Propionibacterium acidi-propionici
atggcagagatgcgtgaagagaaagacagcatgggcacgatcgaggtgccggccgaccactactggggggcccagaccgagcgttcgctccacaacttcgacatcggacgcgacaccttcgtgtggggtcgcgacatggtccgtgcactgggcaccctgaagaagtcggccgcactggccaacaaggaactgggcgaactgccgggcgacgtcgccgacctcatcgtcgcggccgccgacgaggtcatcgcgggcaagctcgacgacgagttcccgctggtggtcttccagaccgggtccggcacccagtcgaatatgaacaccaacgaggtgatctccaaccgcgccatcgagatcgcgggcggcgagaagggctccaagacccccgtccaccccaacgaccacgtcaaccgcggccagtcctccaacgacaccttccccaccgccatgcacatcgcggtggtcaccgagatcaacgagaagctgtacccggctgtcacgcagatgcgcaacaccctcgacgagaaggccaagaagttcgacgacgtcgtcatggtgggccgcacccacctgcaggacgccaccccgatccgcctgggacaggtcatctccggctgggtcgcccagctggacttcgccctcgacggcatccgctacgccgactcccgggcccgcgagctggccatcggcggcaccgccgtcggcaccggtctcaacgcccacccgaagttcggcgagaccgtcgccaagcacgtctccgaggagaccgggctggagttcaagcaggccgagaacctcttcgcctcgctgagcgcccacgacgccctggtgcaggtgtccggctccctgcgggtgctgggcgacgcgctcatgaagatcgccaacgacgtccgctggtacgcatcgggcccccgcaacggcatcggcgagctgctcatccccgagaacgagcccggctcctcgatcatgcccggcaaggtgaacccgacccagtgcgaggccatgaccatggtcgccacccgggtcttcgggaacgacgccaccgtcggcttcgccggatcccagggcaacttccagctcaacgtgttcaagcccgtcatggcccacgcctgcctggagtcgatccgcctgatctcggacgcctgcgtgtccttcgacacccactgcgcctacggcatcgagccgaacatggacaagatcaacgagaacctggacaagaacctcatgcaggtgaccgccctcaaccgccacatcggttacgacctggcctcgaagatcgccaagaacgcccaccaccagggcatctcgctgcgcgagtcggccctgaccgtcggcgggatgaccgccgaggacttcgacaagtgggtcgtccccgcggacatgacgcacccgtccgcggctgagtga

SEQ ID NO: 28 - protein, 468 aa, Propionibacterium acidi-propionici
MAEMREEKDSMGTIEVPADHYWGAQTERSLHNFDIGRDTFVWGRDMVRALGTLKKSAALANKELGELPGDVADLIVAAADEVIAGKLDDEFPLVVFQTGSGTQSNMNTNEVISNRAIEIAGGEKGSKTPVHPNDHVNRGQSSNDTFPTAMHIAVVTEINEKLYPAVTQMRNTLDEKAKKFDDVVMVGRTHLQDATPIRLGQVISGWVAQLDFALDGIRYADSRARELAIGGTAVGTGLNAHPKFGETVAKHVSEETGLEFKQAENLFASLSAHDALVQVSGSLRVLGDALMKIANDVRWYASGPRNGIGELLIPENEPGSSIMPGKVNPTQCEAMTMVATRVFGNDATVGFAGSQGNFQLNVFKPVMAHACLESIRLISDACVSFDTHCAYGIEPNMDKINENLDKNLMQVTALNRHIGYDLASKIAKNAHHQGISLRESALTVGGMTAEDFDKWVVPADMTHPSAAE

SEQ ID NO: 29 - DNA, 1425 bp, Propionibacterium freudenreichii shermanii
atggctgataacaccagcgcgaagacgcgcacggaatccgactccatgggcaccgtcgaggtgccggcaaaccaccattggggggcgcagaccgagcgcagtctgcacaacttcgacatcggtcgtccgaccttcgtgtggggacgcccgatgatcaaggccctcggcatcctgaagaaggctgccgcccaggccaatggagagctcggggagctccccaaggacatctccgagctcatcgtcaaggccgccgatgacgtgatcgccggcaagctcgacgacgacttccccctggtggtcttccagaccggctcgggcacgcagtcgaacatgaacgccaatgaggtgatctccaaccgcgcgatcgagatcgccggcggcgagatgggcaccaagaccccggtgcaccccaatgaccacgtgaaccgtggccagtccagcaacgacaccttccccacggcgatgcacattgccgtggtcaccgagctgcaggagatgtacccgcgcgtgatgaagctgcgcgacaccctggacgccaaggccaaggaatatgacgatgtcgtgatggtggggcgcacccacctgcaggacgcgaccccgatccgcctcggccaggtgatcagcggctgggtggcccagatcgacttcgccctcaagtgcatcaagttctccgacgagcaggcacgcgaactcgccatcggcggcaccgccgtcggcaccggcctgaacgcgcatccgaagttcggcccgctcaccgccgagaagatcagcgacgagaccggcctcaagttcgagcaggccccgaacctgttcgccgcactgagcgcccacgacgcgctggtgcaggtctccggttcgctgcgcgtgctgggcgacgccctgatgaagatcgccaacgacgtgcgttggtatgcctccggcccccgcaatggcatcggcgagctgctgatccccgagaacgagcccggcagctcgatcatgcccggcaaggtcaacccgacccagtgcgaggccatgaccatggtggccaccaaggtgttcggcaacgacgccacggtcggcttcgccggcagccagggcaacttccagctgaacgtcttcaagccggtcatggcctggtgcgtgctggagtccatccagctgctgggcgacacctgcgtgagcttcaacgaccactgtgcggtgggcattgagcccaacctcgagaagatcaagcacaacctcgacatcaacctgatgcaggtgacggcgctcaaccgccacatcggctacgacaaggcctcgaagatcgccaagaacgcccaccacaagggcattggccttcgtgattcggccctcgagctcggcttcctcacccccgaggagttcgacaagtgggtagtgccggccgatatgacccacccgtccgccgccgacgacgactga

SEQ ID NO: 30 - protein, 474 aa, Propionibacterium freudenreichii shermanii
MADNTSAKTRTESDSMGTVEVPANHHWGAQTERSLHNFDIGRPTFVWGRPMIKALGILKKAAAQANGELGELPKDISELIVKAADDVIAGKLDDDFPLVVFQTGSGTQSNMNANEVISNRAIEIAGGEMGTKTPVHPNDHVNRGQSSNDTFPTAMHIAVVTELQEMYPRVMKLRDTLDAKAKEYDDVVMVGRTHLQDATPIRLGQVISGWVAQIDFALKCIKFSDEQARELAIGGTAVGTGLNAHPKFGPLTAEKISDETGLKFEQAPNLFAALSAHDALVQVSGSLRVLGDALMKIANDVRWYASGPRNGIGELLIPENEPGSSIMPGKVNPTQCEAMTMVATKVFGNDATVGFAGSQGNFQLNVFKPVMAWCVLESIQLLGDTCVSFNDHCAVGIEPNLEKIKHNLDINLMQVTALNRHIGYDKASKIAKNAHHKGIGLRDSALELGFLTPEEFDKWVVPADMTHPSAADDD

SEQ ID NO: 31 - DNA, 1407 bp, Propionibacterium acnes
atggcagagatgcgcattgagaaagacagcatgggcgaggtcgaagtacccgccgagcattactggggagcccagacacagcgttccctccacaacttcgagatcggccgtgacaccttcgtttggggccgagacatgatccgtgctctcggaactctcaagaagtccgcggcactagccaacaaggaactgggtgagttgccgggcgacgttgccgacctcatcgtccaggccgccgacgaggtcatcgccggaaaactcgatgacgagttcccgctggtggtcttccagaccggttcgggcacccagtccaacatgaacaccaacgaggtcatcagcaaccgtgcgattgagttggccggtggcgaacgcgggtcgaagaaacccgtccaccccaacgaccacgtcaaccgtggccaatcttccaacgataccttcccgacggccatgcacatcgccgttgtgtgtgccctcaataagcgcctctaccccgccgtccagcagcttcgcgacactctcgacgagaaggccaaaaagtacgacgacgtcgtgatggtcggccgcacccacctgcaggacgcaacgccgatccgcctcggccaggtcattagtggctgggtcgcccaaatcgatttcgccctcgacggcatccgctacgccgattcacgcgcccgtgaactagccatcggcggcaccgccgtcggcaccggcctcaacgcccaccctgatttcggcccgaccgtcgctaagcacgcgaccgaggagactggcattgagttcaagcaggccgacaaccttttcgccgcgctgagcgcccacgacgccctagtacaagtttcggggtcgctgcgtgtcctcgccgacgccctcatgaagattgccaatgacgtccgctggtacgcgtctggcccccgcaacggtatcggcgaactcctgatccccgaaaacgagcccggctcttcgatcatgcctggcaaagtcaatccgacccagtgcgaggccatgacgatggtcgccacccgcgtgttcggtaacgacgcgacagtcggctttgccggttctcaaggcaacttccagctcaacgtgttcaagcccgtcatggcccatgcctgcctggagtcgatccgccttatcgccgattcgtgcatcagcttcgacaaacattgcgcctacggcatcgagccaaaccccgacaaaatcaaggagaacctcgacaagaacctcatgcaggtcacggctctcaaccgtcacatcggttacgacctggcttcgaagatcgctaagaacgctcaccataagggcatcagcctgcgggagtccgctctgacggtcggcggcatgagcgaggaggatttcgacaagtgggtcgtccccgccgacatgactcaccccagcgccgctgaatga

SEQ ID NO: 32 - protein, 468 aa, Propionibacterium acnes
MAEMRIEKDSMGEVEVPAEHYWGAQTQRSLHNFEIGRDTFVWGRDMIRALGTLKKSAALANKELGELPGDVADLIVQAADEVIAGKLDDEFPLVVFQTGSGTQSNMNTNEVISNRAIELAGGERGSKKPVHPNDHVNRGQSSNDTFPTAMHIAVVCALNKRLYPAVQQLRDTLDEKAKKYDDVVMVGRTHLQDATPIRLGQVISGWVAQIDFALDGIRYADSRARELAIGGTAVGTGLNAHPDFGPTVAKHATEETGIEFKQADNLFAALSAHDALVQVSGSLRVLADALMKIANDVRWYASGPRNGIGELLIPENEPGSSIMPGKVNPTQCEAMTMVATRVFGNDATVGFAGSQGNFQLNVFKPVMAHACLESIRLIADSCISFDKHCAYGIEPNPDKIKENLDKNLMQVTALNRHIGYDLASKIAKNAHHKGISLRESALTVGGMSEEDFDKWVVPADMTHPSAAE

SEQ ID NO: 33 - DNA, 843 bp, Selenomonas ruminantium lactilytica
ttgcgagaaatcgacgcaaaacaaatcacggaaaccgtagcacagatgtgcaaagaagcggcctattaccttccggatgacgtctacaacgccatgaagaaggcgcgggagacggaaacttctccggtaggtcagaatgtcctcgaccagattatccgcaatgcggaaatcgccaaggctgaagatcgtccttactgccaggatactggtatgacgattgtcttccttgaagttggtcaggatctgcacattacgggcggtctgttggaagatgcagtcaatgctggcatttccaagggctataccgaaggctacctgcgtaagtccgtggtcggcgagccgctgttcaatcgtgtgaacaccaaggacaatacgcctggcgtcatctacacgaagattgtagcaggtgataagctcaagattaccgtggcaccgaagggctttggttccgagaacaaatccggtgtcaagatgctggtgccggctgatggtgtggaaggtgtgaagaaagcggttatggacattatcctccatgccagcatgaacccctgcccgccgatggttgtcggtgttggtatcggcggtaccatggacagagctgccctcctttccaaactggctctgacgcgttccgttgacgaacgtaatccgatgccggaatacgccaaactggaaggcgaactcctcgaactcatcaatcagacgggtatcggcccccagctgggcggcaatacctcggcactggctgtaaacgtagagtggggccccactcatatcgcaggcctgccggttgcagtaaccatttgctgccatgctatgcgccataaacagcgtgtactttga

SEQ ID NO: 34 - protein, 280 aa, Selenomonas ruminantium lactilytica
MREIDAKQITETVAQMCKEAAYYLPDDVYNAMKKARETETSPVGQNVLDQIIRNAEIAKAEDRPYCQDTGMTIVFLEVGQDLHITGGLLEDAVNAGISKGYTEGYLRKSVVGEPLFNRVNTKDNTPGVIYTKIVAGDKLKITVAPKGFGSENKSGVKMLVPADGVEGVKKAVMDIILHASMNPCPPMVVGVGIGGTMDRAALLSKLALTRSVDERNPMPEYAKLEGELLELINQTGIGPQLGGNTSALAVNVEWGPTHIAGLPVAVTICCHAMRHKQRVL

SEQ ID NO: 35 - DNA, 567 bp, Selenomonas ruminantium lactilytica
atggctgaacagattcggattcagactccgtttacggaggaaatgagccgcaagcttaaagcaggcgatgcggtacttatctctggcgagatcatcgccgctcgtgacgctgcccacaaggccatgacggaagctctggctcggggcgagaaactgccggtagattggcagaatcagatggtctattatctggggccgacgccggctaaaccgggtgatcccattggttcctgcggcccgaccacatccggtcgtatggatgcctacactccgaccatgctggaacagggcatcaagggcatgatcggcaaggggtcccgctccaaagaagtggtggaatccatgaagaagaacggtgtgacctacttcgctgccgttggcggcgccgcagccctcatcgctaaatccgtcaagaagtatgaagtcctggcttatccggaattaggtccggaagcagtggcccgccttacggtggaggacttcccggctatcgtggtcatcgactgcgaaggcaacaacctttacgagacgaatcaggctaagtatcgtacgctgaaaggctactga

SEQ ID NO: 36 - protein, 188 aa, Selenomonas ruminantium lactilytica
MAEQIRIQTPFTEEMSRKLKAGDAVLISGEIIAARDAAHKAMTEALARGEKLPVDWQNQMVYYLGPTPAKPGDPIGSCGPTTSGRMDAYTPTMLEQGIKGMIGKGSRSKEVVESMKKNGVTYFAAVGGAAALIAKSVKKYEVLAYPELGPEAVARLTVEDFPAIVVIDCEGNNLYETNQAKYRTLKGY

SEQ ID NO: 37 - DNA, 2028 bp, Propionibacterium acidi-propionici
atgaccaggccgtcggtcaacaagctcatcggacgactgcacccgagtgggcccgtggccccgaaccgggacctgctcgggccggcccagaagcgggccggataccgggtcggggccgagctgaacgcgaaggtgcccgagggagatccgctgacggcctgggaccgccgccagaacgagtaccggctggtcaacccggccaaccgccggaagatgtcggtgatcgtggtcggcaccggcctgtcgggggcgggagtcgccgccagcctggggcagctcggctaccacgtcgactgcttcagcttccacgactcgccgcgccgggcacactcggtggccgcccaggggggcatcaacgccgcccgcgccaggaaggtcgacggcgacaccctcacccgtttcgtcaaggacaccgtcaagggcggcgactaccgaggacgcgaggccgacgccgtgcggctcggcatcgaatcggtcaaggtcatcgaccacatgtacgccatcggcgcccccttcgcccgcgagtacggcggccaactggccacccggtccttcggcggggtgcaggtctcgcggacctactacacgcgcggcgagaccggacagcagctggaggtggcctgctcccaggccctccaggagcagatcgacgccggcaccgtgacgatgcacaaccgcaccgagatgctcgatctcatcgtggccgacgggagggcccaggggatcgtcacccgcgacctgctgtccggggagatcaggccctggaccgcccacgtcgtcatcctctgcaccggcggatacgggtcggtctaccagtggtcgaccctggccaagggttccaacgccaccgccacctggcgggcccatcgccagggcgcctacttcgccagcccgtgcttcgtccagtttcatcccacggcgctgccggtgagttcgcactggcagtccaagaccaccctgatgagcgagtcgctgcgcaacgacgggcggatctgggtacctaagaaacccggcgacgaccgagagccgaatgagatcggcgaggaggaccgcgactactacctggagcgcaagtaccctgccttcggcaacctcactccccgagacgtggcctcccgcaacgcccgcacccagatcgactcgggccacggcgtcgggccgctgcacaactcggtgtacctggacttccgcgacgccatcgcccggctgggccgcgacaccatcgccgagcgctacggcaacctgttcaagatgtacctcgacgccaccggggaggacccctacgaggtgccgatgagaatcgcacccggggcccacttcacgatgggtgggctgtgggtcgactacaaccagatgagcaccatccccgggctcttcgtcggcggggaggcgtcgaacaactatcacggcgcaaaccggctgggagccaactccctgctgtcggcgtcggtcgacggctggttcaccctgccccggtcggtccccgactacctcgccggattcgtcgggaaggagcccctgagcatcgacgcccccgaggtggacgaggcgatgggccgggtccacgaccgcatcgacaggctgctggccaacgacggctcccaccgccccgaatggttccatcgcaggctcggcgacatcctctacgaccactgcggggtgagccgcgacgagaccggcctggtcgagggcctggaacaggtgcgggcgctgcgcgaggagttctggcgcgacgtccgggtggtcggtgacggggaccgtctcaatcaggaactggagaaggcgggccgggtggccgacttcatcgagctcggcgagacgatgatcctggacgcccttgatcgccgggagtctgccggagcgcatttccggaccgagtacgccacccccgagggggaggccagacgtgacgacgccaactgggccgcggtctcggcctgggagaccacccccgagggggagcatgtccgtcacagcgagccgctcgcgttctcgctgatcgcactgcaggtgagggattaccgatga

SEQ ID NO: 38 - protein, 675 aa, Propionibacterium acidi-propionici
MTRPSVNKLIGRLHPSGPVAPNRDLLGPAQKRAGYRVGAELNAKVPEGDPLTAWDRRQNEYRLVNPANRRKMSVIVVGTGLSGAGVAASLGQLGYHVDCFSFHDSPRRAHSVAAQGGINAARARKVDGDTLTRFVKDTVKGGDYRGREADAVRLGIESVKVIDHMYAIGAPFAREYGGQLATRSFGGVQVSRTYYTRGETGQQLEVACSQALQEQIDAGTVTMHNRTEMLDLIVADGRAQGIVTRDLLSGEIRPWTAHVVILCTGGYGSVYQWSTLAKGSNATATWRAHRQGAYFASPCFVQFHPTALPVSSHWQSKTTLMSESLRNDGRIWVPKKPGDDREPNEIGEEDRDYYLERKYPAFGNLTPRDVASRNARTQIDSGHGVGPLHNSVYLDFRDAIARLGRDTIAERYGNLFKMYLDATGEDPYEVPMRIAPGAHFTMGGLWVDYNQMSTIPGLFVGGEASNNYHGANRLGANSLLSASVDGWFTLPRSVPDYLAGFVGKEPLSIDAPEVDEAMGRVHDRIDRLLANDGSHRPEWFHRRLGDILYDHCGVSRDETGLVEGLEQVRALREEFWRDVRVVGDGDRLNQELEKAGRVADFIELGETMILDALDRRESAGAHFRTEYATPEGEARRDDANWAAVSAWETTPEGEHVRHSEPLAFSLIALQVRDYR

SEQ ID NO: 39 - DNA, 756 bp, Propionibacterium acidi-propionici
atgaaggtcacactggatatctggcgtcaggacgggccgcgggccaagggtcgtttcgagacccacgtcgtcgaggacgccgagcccgagatgagcctgctggagctgctggaccggctcaacgaccagatcgtcgagcagggaggagacccggtcgtcttcgagtccgactgccgcgagggggtgtgcggctcctgcggattcctggtcaacggcgttccgcacggtccggtgcccaacaccccggcgtgccgccagcacctccgcgcattcccgcagatccgacgcttcaagctcgagcccttccgttcggccgccttcccggtgatccgcgatctggcggtcgacaggtcgagtctcgacgccctggtgcgagccggcggaaccgtcaacgtgctcaccggcaccgctcccgacgccgacacggtgccgcagccccatgagcaggccgagcaggccttggacttcgcctcgtgcatcgggtgcggggcctgcgtggccgcgtgccccaacggcgccgcgatgctcttcgccggcgccaagctcgcccacctggcgcggatgccgcagggcaggcaggagcgcggcaggagggcccgccggatggtcgactccctcgacgagttcttcgggccgtgctcgctctacggcgagtgcgcgaaggcctgcccggtggagatccccctcaccgcgatcgccaccgtcaacaaggagcggttgcgcgccggattcagaggtcgcggcagggacgactga

SEQ ID NO: 40 - protein, 251 aa, Propionibacterium acidi-propionici
MKVTLDIWRQDGPRAKGRFETHVVEDAEPEMSLLELLDRLNDQIVEQGGDPVVFESDCREGVCGSCGFLVNGVPHGPVPNTPACRQHLRAFPQIRRFKLEPFRSAAFPVIRDLAVDRSSLDALVRAGGTVNVLTGTAPDADTVPQPHEQAEQALDFASCIGCGACVAACPNGAAMLFAGAKLAHLARMPQGRQERGRRARRMVDSLDEFFGPCSLYGECAKACPVEIPLTAIATVNKERLRAGFRGRGRDD

SEQ ID NO: 41 - DNA, 777 bp, Propionibacterium acidi-propionici
atgagcgtcagccagcaggcgcgcaccgggcgggacgatgtgcaggtccacggggacgaggtggttccccggaacacgcggcgggtcaggccctccaacgtcaccctgaaggtgacgatggccgtgaccggcacgatcttcgcattgttcgtcctggtgcacatgatcggaaacctcaaggccttcatgggccccggggagtacaactcctacgcggcattcctgaggacgctgcttcaccccctcgtcccctacgagggcgtgctgtggatcctgaggatcgtgctgctggcgtgcctggtggcgcacgtctggtccgggatcacgatctgggcgcgcggccggcgcagccgcggaccccatcgccgccagaggatgggcaccctcacctggggggcccgcaccatgctgctctccgggatcctgctgctggccttcgtcgtcgtccacatcctcgacctcaccatcggtgccggggtggcgtcgtcgggataccagccgccggtgcgcacgggcgccgccgaggtggacgtccacgcctaccagaatctggtcgccagcctgtcgcgtccgccgatggcgatcttctacagcctcatcatgctcatcatcggcgtccatctggcccagggcgcctggaacgtcatcaacgacttcgggggcaccggcgccaggctgcgccgggtgtggctgctcatcggaatcctcatcgcactggccatcgtcgtcggcaacggcgcgctgccgatgctcgttctcgcaggggtgatctcatga

SEQ ID NO: 42 - protein, 258 aa, Propionibacterium acidi-propionici
MSVSQQARTGRDDVQVHGDEVVPRNTRRVRPSNVTLKVTMAVTGTIFALFVLVHMIGNLKAFMGPGEYNSYAAFLRTLLHPLVPYEGVLWILRIVLLACLVAHVWSGITIWARGRRSRGPHRRQRMGTLTWGARTMLLSGILLLAFVVVHILDLTIGAGVASSGYQPPVRTGAAEVDVHAYQNLVASLSRPPMAIFYSLIMLIIGVHLAQGAWNVINDFGGTGARLRRVWLLIGILIALAIVVGNGALPMLVLAGVIS

SEQ ID NO: 43 - DNA, 2085 bp, Propionibacterium freudenreichii shermanii
gtgaatatcatcaagaatctcttctccggtgcggccggcaaggctgcatcgaccccgtcagccccgaagcctgcccgtgccagtgcgcaccgtccggcctcgcacctgatcggtgaggccgcccgcgaccacctgggcccggcccagaaggccgccggctatgaggtcggtgccgagatcgacgggcacgtccccgccggcgatgtgctccacacctgggagcaccgtcaggacgactaccgactagtcaacccggccaaccgtcgcaagatgaaggtcatcgtcgtgggctccggcctgtccggtgcgggcttcgcggccagcttcggccagctcggctatgacgtcgactgcttctgtttccatgattcgccgcgtcgcgcccactccgtggcggcgcagggcggcatcaacgccgctcgtgcccgcaaggtcgacggtgacacgctgaagcgcttcgtcaaggacaccgtcaagggcggcgactaccggggccgtgaggccgacgtggtgcgccttggtacggagtcggtgcgtgtcatcgaccacatgtacgcgatcggtgcccccttcgcccgtgaatacggcggtcagctcgccacccgttccttcggtggcgtgcaggtctcgcgtacctattacacgcgcggcgagaccggccagcagatggagatcgcctgttcccaggcgctccaggagcagatcgacgccggcaccgtgaagatgcacaaccgcaccgagatgcttgacctgatcgtcaaggacggccgtgcccagggcatcgtcacccgcgatctgctgaccggcgagatcaaggcctggacggcccatgtcgtggtgctgtgcaccggcggctacggctcggtctaccactggtccacgctggccaagaactcgaatgcaaccgccacctggcgtgcgcacaagcagggcgcgtacttcgcgagcccgtgcttcctgcagttccaccccacggcgcttccggtcagttcacactggcagtcgaagaccacgctgatgagtgagtccctgcgcaatgacggacgcatctgggtgccgaagaaggccggcgacgatcgcccggccaatgacatccccgagaacgagcgcgactactacctggagcgcaagtacccggcattcggcaacctgacgccccgtgacgtcgccagccgcaacgcccgcacgcagattgacagcgggcacggcgtggggccgctgcacaactcggtgtacctcgacttccgcgacgccatcaagcgtctcggcaaggagaccatcgccgagcgctacggcaacctgttcgacatgtacctcgacgccaccggtgagaacccctatgaggtgcccatgcgcatcgcaccgggtgcccacttctcgatgggtggcctgtgggtcgactacgaccagatgagcaacctgcccggtctgttcgtcggcggagaggcatcgaacaactaccacggtgcgaaccgcctgggtgccaactccctgttgtccgcctccgtggatggctggttcaccctgccgctgtcggtgccgaactacctcgccgactatgtcggcaagccgccgctggccgtgcaggatccggccgtcaaggatgccctgggccgggtgcaggatcgcatcaatgccttcctcaccagcaagggcacgcatcgtcccgagtggttccatcgcaagcttggcgacatcctctacgcctactgtggcgtgagccgtgacgaggcgggcctgaccaagggcctcgccgaggtgcgggcactgcgcaaggagtactggaacgacgtcaaggtcgtcggcgacgaccaccggctcaaccaggaactcgagaaggccggccgcgtggccgacttcatcgagctcgccgaggtcatgatcctcgacgccctggaccgccgcgagtcggccggtgcccacttccgtaccgagtacgccactcccgagggagaggccaagcgcaacgacgccgattggtgcgccgtctcggcctgggagacccgccccgatggggttcatgtccgtcacagcgagcccctggaattctcgctgatcgatctgcaggtgagggattaccgatga

SEQ ID NO: 44 - protein, 694 aa, Propionibacterium freudenreichii shermanii
MNIIKNLFSGAAGKAASTPSAPKPARASAHRPASHLIGEAARDHLGPAQKAAGYEVGAEIDGHVPAGDVLHTWEHRQDDYRLVNPANRRKMKVIVVGSGLSGAGFAASFGQLGYDVDCFCFHDSPRRAHSVAAQGGINAARARKVDGDTLKRFVKDTVKGGDYRGREADVVRLGTESVRVIDHMYAIGAPFAREYGGQLATRSFGGVQVSRTYYTRGETGQQMEIACSQALQEQIDAGTVKMHNRTEMLDLIVKDGRAQGIVTRDLLTGEIKAWTAHVVVLCTGGYGSVYHWSTLAKNSNATATWRAHKQGAYFASPCFLQFHPTALPVSSHWQSKTTLMSESLRNDGRIWVPKKAGDDRPANDIPENERDYYLERKYPAFGNLTPRDVASRNARTQIDSGHGVGPLHNSVYLDFRDAIKRLGKETIAERYGNLFDMYLDATGENPYEVPMRIAPGAHFSMGGLWVDYDQMSNLPGLFVGGEASNNYHGANRLGANSLLSASVDGWFTLPLSVPNYLADYVGKPPLAVQDPAVKDALGRVQDRINAFLTSKGTHRPEWFHRKLGDILYAYCGVSRDEAGLTKGLAEVRALRKEYWNDVKVVGDDHRLNQELEKAGRVADFIELAEVMILDALDRRESAGAHFRTEYATPEGEAKRNDADWCAVSAWETRPDGVHVRHSEPLEFSLIDLQVRDYR

SEQ ID NO: 45 - DNA, 759 bp, Propionibacterium freudenreichii shermanii
atgaaggtcacattggatatctggcgccaggcaggtcctcgcgccaagggtgagttcgaaaactacgtcgtcaacgacgctgagcccgagatgagcatccttgagttgctcgatcgactcaacgaccagatcatcgaacagggcggcgagcccgtcgtcttcgagtctgattgtcgtgagggcgtgtgtgggtgctgtggcttcctggtcaatgggaagccccacggtccgctggccaatacgccggcctgtcgccagcacctgcgcgccttccccgaggtgacgcacttcaagttggagcccttccgctccaatgcgttcccggtgatccgcgacctggcgatcgaccgcaccgccctggatgagctcatccaggccggcggcaccgtcaacgtgatgaccggcaccgctccggacgccgacaccagcccccagccgcaccaggtggccgagctcgcgctcgacttcgccagctgcatcggctgcggagcctgcgtggccgcctgcccgaatggttcggcgatgctgttcgccggcgccaagctggcgcatctggcgaagatgccccagggcaaggagcagcgcagctcgagggcgcgtcgcatggtggcagagctcgatgaggacttcggtccctgctcgctgtacggcgagtgcgccatctcctgcccggccggcatctcgctgaccgcgatcgccaccgtgaacaaggagcgctggcgttccgtgttccgcgggcgccactcgcaggacaactga

SEQ ID NO: 46 - protein, 252 aa, Propionibacterium freudenreichii shermanii
MKVTLDIWRQAGPRAKGEFENYVVNDAEPEMSILELLDRLNDQIIEQGGEPVVFESDCREGVCGCCGFLVNGKPHGPLANTPACRQHLRAFPEVTHFKLEPFRSNAFPVIRDLAIDRTALDELIQAGGTVNVMTGTAPDADTSPQPHQVAELALDFASCIGCGACVAACPNGSAMLFAGAKLAHLAKMPQGKEQRSSRARRMVAELDEDFGPCSLYGECAISCPAGISLTAIATVNKERWRSVFRGRHSQDN

SEQ ID NO: 47 - DNA, 756 bp, Propionibacterium freudenreichii shermanii
atgagtgtcggtctgacgacatcaggagggcaaggggacgttgtaacgcgtcacaagctgaagcaacgtccgtccaatgtgactctgaaggtcaccatggcggtgactggaacgatcttcgccctgttcgtctttgtgcacatggtcggaaatctcaaggcttttatgggccctgaagattacgacgcctacgcccgcttcctgcgcaccctgctgtatccgctgctgccctatgagggtggtctgtggatcttccgcctggtgctgtcagcctgcctggtgctgcacgtctgggccggcattaccgtctggctgcgtggccgtaaggctcgtggcaagttcggtcgttacggcgccaagcccaagtccttcttcgctcgcacgatgatcctgtcgggcctgctgatcctggtcttcgtggtggtccacctgctcgatctcacgatcggcgccggactgtcctcgcagtactaccagcctgccgtccacctcggtggcgaccaggtccagatccatgcctacgagaacctcgtggccagcctgtcccgtccctggatggcgatcttctactccgtgatcatggtgatcatcggatgccatatcggccagggtgcctggaacacgatcaatgacttcggtggcaccggcccccgtcttcgcaaggtctggttcctcatcgggctcctcattgcgctggccatcgtcgtggccaacggtgcactccccatgctcatcctcgctggagtgatctcgtga

SEQ ID NO: 48 - protein, 251 aa, Propionibacterium freudenreichii shermanii
MSVGLTTSGGQGDVVTRHKLKQRPSNVTLKVTMAVTGTIFALFVFVHMVGNLKAFMGPEDYDAYARFLRTLLYPLLPYEGGLWIFRLVLSACLVLHVWAGITVWLRGRKARGKFGRYGAKPKSFFARTMILSGLLILVFVVVHLLDLTIGAGLSSQYYQPAVHLGGDQVQIHAYENLVASLSRPWMAIFYSVIMVIIGCHIGQGAWNTINDFGGTGPRLRKVWFLIGLLIALAIVVANGALPMLILAGVIS

SEQ ID NO: 49 - DNA, 2133 bp, Propionibacterium acnes
atgaataactggattgaggccgtcaagaagcctctcgagatgattactgggcgtcatgcgggcgctagacccgaggagcctggcgctgaactagtgaaacccgagcagctccagcccagacgggctgagtcgtcgtcggtggcgagacgggctgccaagggtcccaaggctcgtgacattctcggcccggcgcagaagaaggccggctaccttgttggtgacgaattggacggcaaggctcccgagggggatccactcaccgcgtggacccgtcgtcaaagcgagtacaagctcgtcaatccggcgaaccgtcgcaagatgaacgtcatcgtggtgggaaccggcctctctggttccggggttgccgcaaccctcggccagctcggctaccacgtcgacgttttttgcttccacgattccccgcgtcgagcccactcagttgctgcccagggaggtatcaacgctgcacgtgcccgcaaggttgacggcgattcccttaagaggttcgtcaaggacaccgtcaagggtggtgactaccggggccgagaggccgatgttgttcgactcggcaccgaatcggtacgggttattgaccatatgtacgccatcggcgcgccgttcgcccgagaatatggcggtcagttggctactcgatctttcggaggcgtgcaggtttcgcgcacctactacacccgcggggagaccggtcagcagctcgaagtagcctgctcccaggccctccaggcacaaattgatgccggttcggtgacgatgcacaaccgcaccgagatgcttgacctcatcgttgctgacggtacggctcagggcatcgtcacccgtgaccttctcaccggcgagatcaaggcatggacggcccacgtcgtcatcctatgcaccggcggatatggatcggtgtaccactggtccaccttggccaaagggtccaacgccaccgcgacgtggcgtgctcaccgtcaaggtgcatacttcgccagcccctgcttcgtgcagttccacccgaccgcgctgcccgtcagttcgcactggcagtccaagaccacgctcatgagcgagtcgttgcgcaacgacggccggatctgggtgcccaagaaagccggcgatgatcgtccagccaatgacatcccggaagaggagcgcgactactatttggagcgcaagtacccggccttcggaaacctaacgccgcgcgacgtcgcctcccgcaatgctcggactcagatcgagagcggccacggagtgggcccgcttaagaactcggtgtacctggatttccgtgatgccattgagcgtctaggaaagaagacgattgccgagcgctatggcaacttgttcgacatgtatctcgacgccacgggggagaatccctacgaggtgcccatgcgcatcgccccaggagcccattttacgatgggcggtctgtgggttgactacgaccagatgagtaccatcccgggtctgttcgtcggtggtgaggcgtcaaacaactaccacggcgcgaaccgattgggggctaactccttgctgtcggccagtgtcgacgggtggttcaccctgccactgacggtgccgaactatttggctgggttcgtcggaaagcccgtgttacccctggacgcccccgaggtcagcgcagccattgagagggtgcagaagcgcactgatgccttgctcaactgcggcggaacacaccgcccggaatggttccaccgcaagctcggcgacatcctctatgaggggtgcggggtcgcccgcgacgaggccgggctgctcgacgcccttacaaaggtgcgcgagctgcgtgaggagttctggcgcgacgtcaaggtggtcggaacgggaaaccgactcaaccaggaactcgagaaggccggtcgagtggccgacttcatcgagctcggcgaggtcatgattcttgacgccctggaccgccgggaatcggcaggtgcacacttccgtaacgagtacgccaccgaagccggtgaagccaaacgtaatgacggtgactggtgtgcggtttcggcatgggagacacgtcctgatggcaccaaggtgcggcatagcgaaccgttgtcgttctcgttgatcgatctgcaggtgagggattaccgatga

SEQ ID NO: 50 - protein, 710 aa, Propionibacterium acnes
MNNWIEAVKKPLEMITGRHAGARPEEPGAELVKPEQLQPRRAESSSVARRAAKGPKARDILGPAQKKAGYLVGDELDGKAPEGDPLTAWTRRQSEYKLVNPANRRKMNVIVVGTGLSGSGVAATLGQLGYHVDVFCFHDSPRRAHSVAAQGGINAARARKVDGDSLKRFVKDTVKGGDYRGREADVVRLGTESVRVIDHMYAIGAPFAREYGGQLATRSFGGVQVSRTYYTRGETGQQLEVACSQALQAQIDAGSVTMHNRTEMLDLIVADGTAQGIVTRDLLTGEIKAWTAHVVILCTGGYGSVYHWSTLAKGSNATATWRAHRQGAYFASPCFVQFHPTALPVSSHWQSKTTLMSESLRNDGRIWVPKKAGDDRPANDIPEEERDYYLERKYPAFGNLTPRDVASRNARTQIESGHGVGPLKNSVYLDFRDAIERLGKKTIAERYGNLFDMYLDATGENPYEVPMRIAPGAHFTMGGLWVDYDQMSTIPGLFVGGEASNNYHGANRLGANSLLSASVDGWFTLPLTVPNYLAGFVGKPVLPLDAPEVSAAIERVQKRTDALLNCGGTHRPEWFHRKLGDILYEGCGVARDEAGLLDALTKVRELREEFWRDVKVVGTGNRLNQELEKAGRVADFIELGEVMILDALDRRESAGAHFRNEYATEAGEAKRNDGDWCAVSAWETRPDGTKVRHSEPLSFSLIDLQVRDYR

SEQ ID NO: 51 - DNA, 759 bp, Propionibacterium acnes
atgagggtggaactggatatctggcgtcagaacggccctgacgccaaaggacattttgagcaccacgttgtcgaagatgccgagccggagtggagcctgctggaattgctcgaccggctcaacgatcagatcgtcgaaaatgacggtgatccgattgttttcgagtctgattgtcgtgagggagtgtgcgggtgctgtgggttcatggtcaacggcaagccccatggtccgctgccgaataccccggcgtgtcgtcagcatttgagggcattcccccatatcacccatttcaaaattgagccattccgctcggcagcctttccggtcatccgtgacctagccatcgaccgtacgagcatggatcacctgatacaggccggtggaacggtcgacgttatgacgggcacggcccctgctgcagattcggtgccgcagccgcatgccgaggccgagcaagctctcgacttcgcatcgtgtattggatgcggagcgtgcgtggcggcttgtccaaatggcgcggcgatgcttttcgccggcgcgaagctgtcgcacttggcgatgatgccgcaggggcgtcaggagcgttcgaagcgtgcgcgacgcatgatcaacgcccttgatgaggagttcggtccgtgttccctgtatggcgagtgcgttgaggcctgcccggtgagcattccgttggtagccgtcgcccgggtcaaccgggagcgttggcgtgccggattccggggagcaggcagtaaggacaactga

SEQ ID NO: 52 - protein, 252 aa, Propionibacterium acnes
MRVELDIWRQNGPDAKGHFEHHVVEDAEPEWSLLELLDRLNDQIVENDGDPIVFESDCREGVCGCCGFMVNGKPHGPLPNTPACRQHLRAFPHITHFKIEPFRSAAFPVIRDLAIDRTSMDHLIQAGGTVDVMTGTAPAADSVPQPHAEAEQALDFASCIGCGACVAACPNGAAMLFAGAKLSHLAMMPQGRQERSKRARRMINALDEEFGPCSLYGECVEACPVSIPLVAVARVNRERWRAGFRGAGSKDN

SEQ ID NO: 53 - DNA, 771 bp, Propionibacterium acnes
atgagtgtgcctgacgttcgagaccgtcgtgaccttgaacgcgggggagatgccgtagcggttcatacgcatagcgcggcacgtccttccaacgtcacattaaaattgatcatggccattacggggacgatattcgcgttattcgtcttcgtccatatggtcggaaacctgaaggcctttatggggccaggagattacaacgcctacgccgagtttctccgaaccgtgttgtacccgctatttcccgtcggtggggttttgtggtgcttccggattgttttgcttgtgtgcctggtcttgcatgtgtgggccggtctgacgatctgggtgagggggcgacgagcacgcggccgattttcccggcataacatgaaggccctgggttggggagcccgcacgatggtgctgtcaggaattgtcatcttggctttcgtcgtggtgcatatcctcgatctgaccttgggcatgggcgtacagtcctcatccttccgggcgccagttaatgagggaactcctgacatccacatcaccgcataccagaacctcgtcgccagcctgtcgcgtccgtggatggcgatcttttacaccgtcgtcatgatcatcattgggctgcacatcgcccagggcgtacgtaacaccatcaatgattttggcggtacaggacgccgactgcgcgctatttggacggtgattggtctcctcatcgcgctggccattgtggtgtgcaacggcgcacttcccatgctcattcttgccggggtgatctcatga

SEQ ID NO: 54 - protein, 256 aa, Propionibacterium acnes
MSVPDVRDRRDLERGGDAVAVHTHSAARPSNVTLKLIMAITGTIFALFVFVHMVGNLKAFMGPGDYNAYAEFLRTVLYPLFPVGGVLWCFRIVLLVCLVLHVWAGLTIWVRGRRARGRFSRHNMKALGWGARTMVLSGIVILAFVVVHILDLTLGMGVQSSSFRAPVNEGTPDIHITAYQNLVASLSRPWMAIFYTVVMIIIGLHIAQGVRNTINDFGGTGRRLRAIWTVIGLLIALAIVVCNGALPMLILAGVIS

SEQ ID NO: 55 - DNA, 1908 bp, Selenomonas ruminantium lactilytica
atggctaataaacctgaaaaaaagattattgtcgtaggcggcggcctttcgggcctcatggctacgctgaaaatctgcgaagacggcggtaaggttgacctcttctcttattgcccggttaagcgctcccactctctgtgcgcacagggcggcatgaacgcctgcatggataccaagggcgagcacgattccatctatgagcacttcgatgatacggtatacggcggtgacttcctggctgaccagctggctgtcaaaggcatggttgaggcagctccgaagctcgtgcacatgtttgaccgcatgggcgtgcccttcacccgtacgccggaaggtgttcttgacctccgtaacttcggtggccagaagaacaagcgtactgtattcgccggctccaccacgggccagcagctcctctatgctctcgacgagcaggttcgccgttgggaagttaagggcggcgtaaagaaatatgaattctgggaattcatcaagatcatcaagaacaaagacggtgtttgccgtggtatcgttgcgcagaacatgaactccaacgagatccaggcattcccggctgatgtcgtaatcctggcaacgggcggccctggccaggtatatggccgctgcacggcttctaccatctgcaacggttctgcagtatccgctgtttaccagcagggcgcagaaatcggtaaccccgagttcctgcagatccatccgacggctattccgggttccgataagaaccgcctgatgtccgaagcctgccgtggtgaaggcggccgcgtctgggtataccgcaagaacccccagacgggcgaaaaagaacgctggtacttcctcgaagacatgtatccggcatacggcaacctggtaccccgtgacgttgcgtcccgtgccatctacaaggtcgttgtgcatatggggctcggcatgaccaatccgaaccgcgtatacctcgatctgtcccatattccggctgattacctggagcgcaaactgggcggtatcctcgaaatgtacgatgatttcgtaggtcaggatccccgtaaggttcccatggaaatcttcccgtccatccattactccatgggtggcatctgggtagacagagagcatcacaccaatatcccgggcctcatggcttccggcgagtgcgattaccagtaccacggtgcaaaccgtctcggtgcaaactccctgctgtccgctacttactccggcaccatctccggtccggaagctctgcgccttgcccgcagcggcaagctgggtgatgctctcaccaacgaagagctggaagcagcccgcaaagagtgcgtagaggaattcgacaagatccgcaacatgaatggtgctgaaaatgctcatcagatgcaccatgaactgggcgacatcatgtacaaatatgtttccatcgaacgcgataacaacggcctcaaacagtgcatgaaagaactccatgccctgctcaaacgttgggacaatatcggtgttacggatcatggcaactgggcaaaccaggaagcaatgttcgtgcgccagctccgcaacatgatcatctacgctatggccatcaccaagtccgctctgcagcgtgacgaaagccgcggtgcccatgcgaagatcgtcctcaagtccgactatgatagctgggatgcagccaagaagaaggcctttgacgagaagaatggcaaataccatttcgatgctgaaactggccgtgctatgacggatgatggcaatgatgacctgctgttctttggccgtgatgatgagaaattcatgcgcaccacggtggtatcttttgacgctgccaacagtgaaccggaagtttcctatcgtgaattcgagcactctctcattaagccgcgtctgcgtaactacgccgtagctaagaaagagtaa

SEQ ID NO: 56 - protein, 635 aa, Selenomonas ruminantium lactilytica
MANKPEKKIIVVGGGLSGLMATLKICEDGGKVDLFSYCPVKRSHSLCAQGGMNACMDTKGEHDSIYEHFDDTVYGGDFLADQLAVKGMVEAAPKLVHMFDRMGVPFTRTPEGVLDLRNFGGQKNKRTVFAGSTTGQQLLYALDEQVRRWEVKGGVKKYEFWEFIKIIKNKDGVCRGIVAQNMNSNEIQAFPADVVILATGGPGQVYGRCTASTICNGSAVSAVYQQGAEIGNPEFLQIHPTAIPGSDKNRLMSEACRGEGGRVWVYRKNPQTGEKERWYFLEDMYPAYGNLVPRDVASRAIYKVVVHMGLGMTNPNRVYLDLSHIPADYLERKLGGILEMYDDFVGQDPRKVPMEIFPSIHYSMGGIWVDREHHTNIPGLMASGECDYQYHGANRLGANSLLSATYSGTISGPEALRLARSGKLGDALTNEELEAARKECVEEFDKIRNMNGAENAHQMHHELGDIMYKYVSIERDNNGLKQCMKELHALLKRWDNIGVTDHGNWANQEAMFVRQLRNMIIYAMAITKSALQRDESRGAHAKIVLKSDYDSWDAAKKKAFDEKNGKYHFDAETGRAMTDDGNDDLLFFGRDDEKFMRTTVVSFDAANSEPEVSYREFEHSLIKPRLRNYAVAKKE

SEQ ID NO: 57 - DNA, 753 bp, Selenomonas ruminantium lactilytica
atggcagaacagaaaaaagtcagattcataatcgagcgtcaggatggcccggatacggccccctacacgcaggaattcgacgtagattaccgtccgggcctcaacgttgttgccgccctgatggaaatccagaagaacccggtcaccgttgacggcaagaaagttgctcctgttgtttgggaatgcaactgcctggaaaaagtctgcggtgcctgcatgatggttatcaatggtaaggctcgtcaggcttgctgctccctgattgacaatctggaacagcccatccgcctgcagccggcccgtacgttcccggttatccgcgacctgctcatcgaccgctccgtgatgtttgaaagcctcaaacgcattcagggctgggtggaagtggatggctcctgggaagtcaaggatgccccgatccagaacccgtacaccgcacagacggcttacgagatttctcactgcatgacctgcggttgctgcatggaagcatgccccaacgttggtccgcagtccgacttcatcggcccgtccccgacggtacaggcatatctcttcaatctccatccgctgggaaaattcgacgctccgaagcgcctgaatgccctgatggaaaaaggcggcatcaccagctgcggcaacagccagaactgcgtacaggcctgcccgaagaacatcaagctgacgacttacctcgcacagctcaaccgcgatgtcaacaaacaggctctgaagaatatcttcaaccactaa

SEQ ID NO: 58 - protein, 250 aa, Selenomonas ruminantium lactilytica
MAEQKKVRFIIERQDGPDTAPYTQEFDVDYRPGLNVVAALMEIQKNPVTVDGKKVAPVVWECNCLEKVCGACMMVINGKARQACCSLIDNLEQPIRLQPARTFPVIRDLLIDRSVMFESLKRIQGWVEVDGSWEVKDAPIQNPYTAQTAYEISHCMTCGCCMEACPNVGPQSDFIGPSPTVQAYLFNLHPLGKFDAPKRLNALMEKGGITSCGNSQNCVQACPKNIKLTTYLAQLNRDVNKQALKNIFNH

SEQ ID NO: 59 - DNA, 627 bp, Selenomonas ruminantium lactilytica
atgtttcacacaactttttacgtgcgtcgtctgcattcattagtgggcctcttggcgttgggcatggttctctttgaacatatcttcaccaactccatggctctgggcggcgctcctgcacttaacggagccctggcaatgatggagctcatcccgcatccgatcttcctcggactggaaattggcgctattgcaacgcctctgctcttccatgccatctatggtatctacatctgcctgcaggctaagaacaatccgggccgttatggctatgtccgcaactggcagttcgctctgcagcgctggacggcatggttcctggtagtattcctggtttggcacgtattctatctgcgtatcctgaccaagggcattgccggtgttcccatttcctatgaactcctgcagaactacttcgtagcaaatcctgcctatgctcttctctacatcatcggtatgtttgctgccatcttccatttctgcaatggtatcacgaccttctgcatgacctggggtatcgcgaaaggcccccgcgtccagaatgtggtaagtgcactgagcatgggcctctgcgctgtgctgtgcctcgtgacgctggcatttatgggcagctactttgtgatgtaa

SEQ ID NO: 60 - protein, 208 aa, Selenomonas ruminantium lactilytica
MFHTTFYVRRLHSLVGLLALGMVLFEHIFTNSMALGGAPALNGALAMMELIPHPIFLGLEIGAIATPLLFHAIYGIYICLQAKNNPGRYGYVRNWQFALQRWTAWFLVVFLVWHVFYLRILTKGIAGVPISYELLQNYFVANPAYALLYIIGMFAAIFHFCNGITTFCMTWGIAKGPRVQNVVSALSMGLCAVLCLVTLAFMGSYFVM

SEQ ID NO: 61 - DNA, 1512 bp, Propionibacterium acidi-propionici
atgtcagatcggattgccaacgaggctctgcgtcagaaggtgatgagtgccgatgacgcggcttccctcatccatgacggcgaccagatcggcttcggtgggttcaccgggtcgggctaccccaaggagttcccgccggcccttgccaagcgcatcacggccgcccacgagaagggcgagcacttcaccgtcaacgccttcaccggcgcctccaccgcccctgagctcgacggggtcctggccggggtggacggcatcgggatgcgctccccgtaccagtcggaccccacgatgcgggccaagatcaacgacggcacgagcttctacaccgacatccacctgtcgcagttcggcatgcaggtccgtgagggattcttcggcaagctggactacgccgtgatcgaggccaccaagatcaccgccgacggcaacgccatccccacctcctcggtcggcaacaatgccgtctacgtcgagaaggccgagaagatcatcatcgaggtcaacgactggcagtccgaggacctcgagggcatgcacgacatctactacggattcgcgctgccgccgaaccgcgtcccgatcccgatcacccatcccggtgaccggatcggcgagaccttcctgcgggtgccgcagaacaaggtcgtcgcgatcatcgagacccacgatcccgaccgcaactcgcccttcaagccgatcgacgaggactcccacaagatcgccggctacctgctggacttctacgccaatgaggtcgagcacggccggatgccgaagaacctgctgcccctgcagtcgggcgtcggcaatatcccgaacgccgtcctcgacgggctgctgcactcggatctggatcacctcacctcctacaccgaggtgatccaggacgggatgatcgatctcatcgacgccggcaaggtcgacgtggcctcggccacggccttctcgctgtctcccgactatgcccacaagatgaatgagaacgcggccttctaccgcaaccacatcatcctgcgcccccaggagatctcgaaccatcccgaggtgatccgccgtctcggcgtgctgggcgccaacggcatgatcgaggccgacatctacggcaacgtgaactccacccacgtgatgggctcgcggatgatgaacggcatcggcgggtcgggagacttcacccgcaacgccttcatctcggccttcgtgtccccctcgaccgccaagggcggcaagatctcggcgatcgtgccgatggtctcccacgtcgaccacaccgagcacgacacgatggtcatcatcaccgagcagggcatcgccgatctgcgtggcctggccccccgccagcgcgccccgaagatcatcgacaactgcgctcacccggactaccgggatgcgctgcacgactactacgaccgggccctgcgcgactgcaagttcaagcagaccccgcacctgctcgaggagtcctactccttccaccgcaggttccaggagaccggctccatgaaggcctga

SEQ ID NO: 62 - protein, 503 aa, Propionibacterium acidi-propionici
MSDRIANEALRQKVMSADDAASLIHDGDQIGFGGFTGSGYPKEFPPALAKRITAAHEKGEHFTVNAFTGASTAPELDGVLAGVDGIGMRSPYQSDPTMRAKINDGTSFYTDIHLSQFGMQVREGFFGKLDYAVIEATKITADGNAIPTSSVGNNAVYVEKAEKIIIEVNDWQSEDLEGMHDIYYGFALPPNRVPIPITHPGDRIGETFLRVPQNKVVAIIETHDPDRNSPFKPIDEDSHKIAGYLLDFYANEVEHGRMPKNLLPLQSGVGNIPNAVLDGLLHSDLDHLTSYTEVIQDGMIDLIDAGKVDVASATAFSLSPDYAHKMNENAAFYRNHIILRPQEISNHPEVIRRLGVLGANGMIEADIYGNVNSTHVMGSRMMNGIGGSGDFTRNAFISAFVSPSTAKGGKISAIVPMVSHVDHTEHDTMVIITEQGIADLRGLAPRQRAPKIIDNCAHPDYRDALHDYYDRALRDCKFKQTPHLLEESYSFHRRFQETGSMKA

SEQ ID NO: 63 - DNA, 1524 bp, Propionibacterium freudenreichii shermanii
tcaacggtagtgcgaaagcttcatcgtgccgttctcgatgaagttcaggtgccaatcgtaggaatggcgcaggtcgtgcggcgtgtgcttggccttcgacgtcaccagcgagtggtcgaagtactcctgcagctgcgcacggaaatcgggatgcacgcaattgtcgatgatcttctgggcgcgcaggcgcggtgccaggccacggaggtcggcgatgccctgctcggtgatgatcaccatcacgtcatgctcggtgtggtcgacgtggctcaccatcggcacgatcgccgagatggcgccgtccttggcggtcgacggcgacacgaatgccgagatgtaggcgttgcgcgtgaagtcaccggaaccaccgatgccgttcatcatgcgggtgcccatcacgtgggtggagttcacattgccgtagatgtcggcctcgatcatgccattgcacgacagcacgcccaggcgacggatgacctcaggatggttcgagatctcctggggacgaagaacgatcgacttgcggtagttcttcgcgttctcattcatgttgtgcgcgtaatcggggctcagcgagaaggccgttgccgaggcgacggccagcttgccggcgtcgatcaggtcgaccatgccgtcctggatcacctcggtgtagctggtgaggttctcgaggtcggaatgcagcaggccgtcgagcacggcgttggggatattgcccacgccggactgcagcggcagcaggttcttcggcaggcggccgtgcttgacctcgttggcatagaagtcgagcaggtagccggcaatggcgcgcgaatcgtcgtcaatcggcttgaacggcgagttgcggtcacggtcggtggtctcgatgaccgccacgaccttgtcggaatcgatggtcaggaaggtgtcgccgatgcggtcgcccacattgttgatcgggatgatcggacggttcgggggagtcagatatccattccagatatcgtgcattccctcgaggtcgggcgactgccaggagttcacctcgatgatgatcttctcggccatgtcgaggtaggtcttgttgttgcccaccgacgaggtgggaacgatgttcccgtcctcggtgatgcgcacggcctcgaccacggcgaagtcgagcttgcccatgaagccctcggccaccagctgggccgagtgcgacaggtggatgtcggtgtacagcgtggtgccgtcgttgatcttcttgcgcagcgtcggatccgactggtacggcatgcggtagtgcatgccatcgacgccggccagggcaccatcgagctcgggagcggtggacgcaccggtgaacgcgttgacggtgaattcctcgccgcgctcgtgggctgccttgatcctgttggccagggcctggggcagttccttcgggtaacccgagccggtgaaaccgccgaagccgatctgggctccattggggataagggctgcggcctcatcggcggtcatgatctttccgcgcaacttctcgttggagatgcgttcgttcat

SEQ ID NO: 64 - protein, 507 aa, Propionibacterium freudenreichii shermanii
MNERISNEKLRGKIMTADEAAALIPNGAQIGFGGFTGSGYPKELPQALANRIKAAHERGEEFTVNAFTGASTAPELDGALAGVDGMHYRMPYQSDPTLRKKINDGTTLYTDIHLSHSAQLVAEGFMGKLDFAVVEAVRITEDGNIVPTSSVGNNKTYLDMAEKIIIEVNSWQSPDLEGMHDIWNGYLTPPNRPIIPINNVGDRIGDTFLTIDSDKVVAVIETTDRDRNSPFKPIDDDSRAIAGYLLDFYANEVKHGRLPKNLLPLQSGVGNIPNAVLDGLLHSDLENLTSYTEVIQDGMVDLIDAGKLAVASATAFSLSPDYAHNMNENAKNYRKSIVLRPQEISNHPEVIRRLGVLSCNGMIEADIYGNVNSTHVMGTRMMNGIGGSGDFTRNAYISAFVSPSTAKDGAISAIVPMVSHVDHTEHDVMVIITEQGIADLRGLAPRLRAQKIIDNCVHPDFRAQLQEYFDHSLVTSKAKHTPHDLRHSYDWHLNFIENGTMKLSHYR

SEQ ID NO: 65 - DNA, 1515 bp, Propionibacterium acnes
atgtcagagcggattgccaacgcagccctgcgtcagaaagtgatgagcgcggacgacgcggctgccctcatccatgacggcgaccagatcggattcggtggattcactgggtcgggctaccccaaggaacttccaggcgcactggccaagcgcatccaagagtcccacggccgtggtgagaagttcaccgtcaacgtcttcaccggagcctcgaccgctcctgaactcgacggcgccctcgcctctgtcgacggcatcggctggcgtatgccgtaccagtccgatccccagatgcgaagcaagatcaacgacggcacctccttctataccgacatccatctgtcggagtcgggcatgatggtgcgtcagggcttctttggcaaggtcgacttcgccgtcatcgaggccacacgaatcaccgcggacggggatgtcgtcctcacctcgtcggttggcaacaacgcggtctactgtgacaccgccgagaaagtcatcatcgaggtaaattcgtggcagtccgaggacctcgaaggaatgcacgacatctacggtggttttgcgcttcctccgaaccgggtgccgatcccgattacccatcccggtgaccggatcggtgacaagttcctccacatcccccagaacaagattgtggcgatcatcgagaccgcaggccccgaccgcaacaccccgttcaagccgatcgacgacgactctcgcaagatcgccggattcctgctcgacttttatgacaacgaggtcaagcagggacgcatccccaagaacctgctgccgctccagtccggcgtcggcaacatcccgaacgccgttcttgacggcctgcttcactccgacctggagcatctgacctcgtacaccgaggtgatccaggacggcatgatcgacctcatcgacgccggcaagcttgacgtcgcctccgctaccgccttctcgctgtcgcctgactatgcgcacaagatgaacgagaatgcagccttctaccgcgatcacatcattttgcgcccgcaggaaatttcgaaccaccccgaggtcattcgccgcctcggcgtcatcggcgctaacggcatgatcgaggccgacatctatggcaatgtcaactcgacccatgtcatgggctcgcggatgatgaacggcatcggtggatccggcgacttcacccgcaatgcctacatttcggccttcgtgtccccatcgacagccaagggtggcgccatctcggcgatcgtcccgatggtctcccacgtcgaccacaccgagcacgacggcatggttatcatcaccgagcaaggcatcgctgatctgcgtggcctggccccacgccagcgcgcccggaagatcatcgagaactgtgcccacccggactaccgtccgatgctgctcgactactacgagcgtgcgctgcgcgactgcaagttcaagcacaccccgcacctgctgggtgaggcgtactcatggcacacccggttcctcgagaccggcaccatgaagaaggactga

SEQ ID NO: 66 - protein, 504 aa, Propionibacterium acnes
MSERIANAALRQKVMSADDAAALIHDGDQIGFGGFTGSGYPKELPGALAKRIQESHGRGEKFTVNVFTGASTAPELDGALASVDGIGWRMPYQSDPQMRSKINDGTSFYTDIHLSESGMMVRQGFFGKVDFAVIEATRITADGDVVLTSSVGNNAVYCDTAEKVIIEVNSWQSEDLEGMHDIYGGFALPPNRVPIPITHPGDRIGDKFLHIPQNKIVAIIETAGPDRNTPFKPIDDDSRKIAGFLLDFYDNEVKQGRIPKNLLPLQSGVGNIPNAVLDGLLHSDLEHLTSYTEVIQDGMIDLIDAGKLDVASATAFSLSPDYAHKMNENAAFYRDHIILRPQEISNHPEVIRRLGVIGANGMIEADIYGNVNSTHVMGSRMMNGIGGSGDFTRNAYISAFVSPSTAKGGAISAIVPMVSHVDHTEHDGMVIITEQGIADLRGLAPRQRARKIIENCAHPDYRPMLLDYYERALRDCKFKHTPHLLGEAYSWHTRFLETGTMKKD

SEQ ID NO: 67 - DNA, 1506 bp, Selenomonas ruminantium lactilytica
ttagtctttcatcatgctgccattggccacgaatctctcatggaaggacagagcctcttcgagaatatgcggcgtattggcatggtgggttgcttcggttgctctctcgaagtaatccagcagaatcggacgatagtccggatgggcgcagttattgatgatttccaaagcgcgctcacgcggagccttgccacggagatcggcaataccctgttccgtgatgatgatatccacatcatgttccgtgtggtcgatatgggagcacatgggcacaactgcggaaatcttgccgcctttggcaatggacggcgtatagaagatggtaagatatgcgttgcgggcaaagtcgccgctgccgccgataccgttcatcatcttggtacccgtgatatgcgtggagttgacattaccgtagatatccacttcgatagccgtgttcatggcgataacgccgagacggtgaacgacttccgggctgttggagatttcttccggacgcagcaacagatacttcttgtatttggccacatccttatagaaacgtgccatgccttccggggacgggctgaaagccgtaccagaggcaatcagcagcttgccggcatcgatgaggtccaacatgccgtcctggattacttcggtgtaaaccgtcagatccttgaggtcggaatctacgaaccctgccataacagcgttggctacgttgccgacaccagactgcagcggcagcagattcttcggcatgcggccggcttccacttccttcttgaggaagtccagggtgaactggctcatcttgcgggaatcctcatcaatcgccttcagcggacgcgtatggtcaggaatatcgcagggaacgatgtatttgatcttatccggcgtgcaggggatataaggcgtacctacacgatccgtagccttgacgatgggaatgggcaggcggtttggcggatccatcgggatatacacatcatgcatgccttcaagttccagcggctgggaagtattgacttccacgatgaccgtatcggcgctctgcacataggaagcggcattacccagtgatgtcgtcgggatgatattgccctcttccgtgatggcacaggcctctaccaccgccacatccaccttgccaagatagccgcaacggctgagctgggctgactcggaaagatgcagatccagataatctacagaaccgtcgttgatttcattgcgcagatccttatccgtctggtagggtaaacgcttcttgataccatgaactttggccaatgcctcatccagctctgggcctgtggaagctcccgtccagaggttaatggtgaacggctctttcttcatccgttcagctaatgccagtggaatggctttcggatatgccgaggctgtaaaaccactggttgcaacattcatgccttctttgaaaaaagctgctgcttcttccgccgtgacaatcttgctctgcaactctttgttgcgcacgcggtcaagaatatcaatcat

SEQ ID NO: 68 - protein, 501 aa, Selenomonas ruminantium lactilytica
MIDILDRVRNKELQSKIVTAEEAAAFFKEGMNVATSGFTASAYPKAIPLALAERMKKEPFTINLWTGASTGPELDEALAKVHGIKKRLPYQTDKDLRNEINDGSVDYLDLHLSESAQLSRCGYLGKVDVAVVEACAITEEGNIIPTTSLGNAASYVQSADTVIVEVNTSQPLELEGMHDVYIPMDPPNRLPIPIVKATDRVGTPYIPCTPDKIKYIVPCDIPDHTRPLKAIDEDSRKMSQFTLDFLKKEVEAGRMPKNLLPLQSGVGNVANAVMAGFVDSDLKDLTVYTEVIQDGMLDLIDAGKLLIASGTAFSPSPEGMARFYKDVAKYKKYLLLRPEEISNSPEVVHRLGVIAMNTAIEVDIYGNVNSTHITGTKMMNGIGGSGDFARNAYLTIFYTPSIAKGGKISAVVPMCSHIDHTEHDVDIIITEQGIADLRGKAPRERALEIINNCAHPDYRPILLDYFERATEATHHANTPHILEEALSFHERFVANGSMMKD

SEQ ID NO: 69 - DNA, 1908 bp, Propionibacterium acidi-propionici
atgactgatcccgacaacctcgagaagccggttgacgccgacgttcccgaggcgctcaccctggccggtgacttcccgacgcccacgcaggagcagtgggagaaggaggtggccaaggtcttcaaccggggacgccccgagggcaagcagctctccttcgagcagtccctcaagcgcatggagccgaccaccgtcgacggcctccagttcgagcccatgtacacccgggacgatgcgcccgagaagctcggcgctcccggagtggcccccttcacccgcggcaccaccatcaagaccggcgacgccgacgcctgggacgtgcgggccctccacgaggatcccgacaccgaatccaccaggaaggccatcgtcgccgatctggagcgcggcgtcacctccatctggctgcgcgtcggagccgacgcgatcaagcccgccgacatcgccggcgacctcaaggaggtgctgctgaacctggccaaggtcgaggtctccagccgtgacgaccaggaggccgcggccaacgccctcctcgacgtctacgcctcctccgagctcgcgcccgaggagctgtccttcaacctgggcatcgacccgatcgggctggccgccctcaacggcggcgaggccgacctctccggtctgtccgcctggtccgggaagatcaagaactacaagaacgcgcgggccttcgtggccgacggcacgatctaccacaatgccggcgccggagacgtcgccgagctggcctggacgatcgccaccgccgtcgagtacgtgcgcgccctcgtcgagcagggggtctcggcctccgacgccttcgacgcgatcaacttccgggtgaccgccacccacgaccagttcctcaccatcgcccggctgcgcgccctgcggaccgtgtggaaccggatcggcgaggtcttcgaggttcccgaggacaagcgcggcgcccgtcaggaggccgtcaccagctggcgcgagctgacccgtgacgatccctacgtcaacatcctgcgcggcacgatctcgaccttcagcgcggccgtcggcggcgccgaggccatcaccacgctgcccttcgacgcggccatcggcctgccgaagaaccagttcacccgcaggatcgcccgtaacaccggcatcgtgctggccgaggagtgcaacatcgggcgggtcaacgacccggccggcggctccttctacgtcgagtccctcaccaagagcctggagcaggccgcatgggccaagttccaggagatcgaggccgcgggcggattcgccaagttcttggccgacggcaaggtcgcggccgagctcgaggggctcaacgcggaacgggccaagcgcctcgccacccgcaagcagcccatcaccgccgtctccgagttcccgatgatcggggcccgcaccctggaggccaagcccttcccggccgccgccgagcgcaacgggctggcctggcaccgcgacgccgaggtcttcgagggcctcgttgatcgctccggcaaggccaccgaggcgccgaaggtcttcctggcatgtctgggaacccggcgcgacttcggcgcccgcgagggcttctcggccccgatgtggcacatcgccggcatcgagacccccgaggtcgagggcgggtccaccgaggacatcgtcaaggccttcaaggcttcgggcaccaccgtcgtggatctctgctcgaacaagaagacctacgccgcacagggactggaggtcgccaaggcccttcgggaggccggcgccaagaaggtctacctgtcgggtgccttcaaggaattcggtgatgacgccgccgaggccgagcaggtcttcgacggccgtgtcgccatggggatggacgtcgtcgcgaccctctccgacactctggacacgttgggagttgccaagtga

SEQ ID NO: 70 - protein, 635 aa, Propionibacterium acidi-propionici
MTDPDNLEKPVDADVPEALTLAGDFPTPTQEQWEKEVAKVFNRGRPEGKQLSFEQSLKRMEPTTVDGLQFEPMYTRDDAPEKLGAPGVAPFTRGTTIKTGDADAWDVRALHEDPDTESTRKAIVADLERGVTSIWLRVGADAIKPADIAGDLKEVLLNLAKVEVSSRDDQEAAANALLDVYASSELAPEELSFNLGIDPIGLAALNGGEADLSGLSAWSGKIKNYKNARAFVADGTIYHNAGAGDVAELAWTIATAVEYVRALVEQGVSASDAFDAINFRVTATHDQFLTIARLRALRTVWNRIGEVFEVPEDKRGARQEAVTSWRELTRDDPYVNILRGTISTFSAAVGGAEAITTLPFDAAIGLPKNQFTRRIARNTGIVLAEECNIGRVNDPAGGSFYVESLTKSLEQAAWAKFQEIEAAGGFAKFLADGKVAAELEGLNAERAKRLATRKQPITAVSEFPMIGARTLEAKPFPAAAERNGLAWHRDAEVFEGLVDRSGKATEAPKVFLACLGTRRDFGAREGFSAPMWHIAGIETPEVEGGSTEDIVKAFKASGTTVVDLCSNKKTYAAQGLEVAKALREAGAKKVYLSGAFKEFGDDAAEAEQVFDGRVAMGMDVVATLSDTLDTLGVAK

SEQ ID NO: 71 - DNA, 2187 bp, Propionibacterium acidi-propionici
gtgagcaccttgccccgtttcgattcgatcaacctgggtgacgcgcagcctgccgccgacgcccaggagcagttcgccaagctggccgccgcggccggtgagcaggagccctgggtcaccccggagcagatcccggtcgggcatctgtacggcgaggacgtctaccaggacatggactggctgaacacctatgcggggatccccccgttcgtgcacggcccgtacgccaccatgtacgccttccgtccgtggaccatccgccagtacgccggattctccaccgccaaggagtccaacgccttctaccgcgccaacctggccgccggccagaagggcctgtcggtcgccttcgacctgccgacccaccgcggatacgactccgacaacccgcgcgtccccggtgacgtcggcatggccggtgtggccgtggactccatcaaggacatgcgcgagctgttcgcgggcatcccgctggaccagatgagcgtgtcgatgaccatgaacggcgccgtgctgccgatcctggccctctacgtggtggccgccgaggagcagggcgtcaagcccgagcagctcgccggtacgatccagaacgacatcctcaaggagttcatggttcgtaacacctacatctacccgccgcagccctcgatgcggatcatcgccgacatcttcgcctacaccagcgcgaacatgccgaagtggaactccatctcgatctccggctaccacatgcaggaggccggcgccaccgccgacatcgagatggcctacaccctggccgacggcgtcgactacatccgcgccggtgagaacgtcggcctgaaggtggaccagttcgcaccgcggctgtccttcttctgggctatcgggacgaacttcttcatggaggtcgccaagatgcgcgcggcccgcatgctgtgggccaagctggtgcaccagttcgggccgaagaacccgaagtccatgagcctgcgcacccactcgcagacctcgggctggtcgctgaccgcccaggacgtctacaacaacgtggtgcgcacctgtgtggaggccatggccgccacccagggccacacccagtccctgcacaccaactcgctggacgaggccatcgccctgccgaccgacttctcggcccgcatcgcgcgctccacccagcagttcctgcagcaggagtcgggcaccacccgggtcatcgacccctggagcggctcggcctacgtcgagaagctcaccctggagctggcccgcaaagcgtggggccacatccaggaggtcgaggccgccggcggcatggccaaggccatcgagaagggcatcccgaagatgcgcatcgaggaggccgcggcccgcacccaggcccgtctggactccggccgtcaggccctcatcggtgtcaacaagtaccagctggatgaggacgagcccctcgaggtccgcaaggtcgacaactcccaggtgctggccgagcagaaggccaagcttgaggcgctgcgcgccgagcgcgacagcgcggcctgcgccaaggctctggaggatctcacctgggccgccgccaaccccgatccgaccgaccctgaccgcaacctgctgaagatgtgcatcgacgccggtcgcgccggagcgtcgctcggtgagatgagtgacgcgatggagaaggttttcgggcgctacaccgctcagatccatacaatctccggtgtgtacagcaaggcagccggatcctccgaggccacctcgaaggtccagggaatggtcaaggagttcgaggagaaggagggccgccgcccccgcatcctcatcgccaagatgggccaggacggccacgaccgcggtcagaaggtcgtcgcgacggcctatgccgacctcggcatggacgtcgacgtcggcccgctcttccagactcccgaggagactgctcggcaggctgtcgagggcgacgtccacgtcgtcggcgtctcctccctggcggccgggcacctcaccctggtgcccgagctgcgcgaggagctggccaagctgggtcgttccgacatcatgatcgtggtcggcggcgtcatcccgaccggcgacttccaggaactccgcgatgacggcgccgtggcgatctacccgcccggcaccaacatcccggaggccgcggtcgacctcatgaccaagctgctcgagcacgtcgaggactga

SEQ ID NO: 72 - protein, 728 aa, Propionibacterium acidi-propionici
MSTLPRFDSINLGDAQPAADAQEQFAKLAAAAGEQEPWVTPEQIPVGHLYGEDVYQDMDWLNTYAGIPPFVHGPYATMYAFRPWTIRQYAGFSTAKESNAFYRANLAAGQKGLSVAFDLPTHRGYDSDNPRVPGDVGMAGVAVDSIKDMRELFAGIPLDQMSVSMTMNGAVLPILALYVVAAEEQGVKPEQLAGTIQNDILKEFMVRNTYIYPPQPSMRIIADIFAYTSANMPKWNSISISGYHMQEAGATADIEMAYTLADGVDYIRAGENVGLKVDQFAPRLSFFWAIGTNFFMEVAKMRAARMLWAKLVHQFGPKNPKSMSLRTHSQTSGWSLTAQDVYNNVVRTCVEAMAATQGHTQSLHTNSLDEAIALPTDFSARIARSTQQFLQQESGTTRVIDPWSGSAYVEKLTLELARKAWGHIQEVEAAGGMAKAIEKGIPKMRIEEAAARTQARLDSGRQALIGVNKYQLDEDEPLEVRKVDNSQVLAEQKAKLEALRAERDSAACAKALEDLTWAAANPDPTDPDRNLLKMCIDAGRAGASLGEMSDAMEKVFGRYTAQIHTISGVYSKAAGSSEATSKVQGMVKEFEEKEGRRPRILIAKMGQDGHDRGQKVVATAYADLGMDVDVGPLFQTPEETARQAVEGDVHVVGVSSLAAGHLTLVPELREELAKLGRSDIMIVVGGVIPTGDFQELRDDGAVAIYPPGTNIPEAAVDLMTKLLEHVED

SEQ ID NO: 73 - DNA, 1917 bp, Propionibacterium freudenreichii shermanii
atgagcagcacggatcaggggaccaaccccgccgacactgacgacctcactcccaccacactcagcctggccggggatttccccaaggccactgaggagcagtgggagcgcgaagttgagaaggtactcaaccgtggtcgtccaccggagaagcagttgaccttcgccgagtgtctgaagcgcctgacggttcacaccgtcgatggcatcgacatcgtgccgatgtaccgtccgaaggacgccccgaagaagctgggttaccccggcgtcgcacctttcacccgcggcaccacggtgcgcaacggcgacatggatgcctgggacgtgcgcgccctgcacgaggatcccgacgagaagttcacccgcaaggcgatcctcgaaggcctggagcgtggcgtcacctccctgttgctgcgcgttgatcccgacgcgatcgcacccgagcacctcgacgaggtcctctccgacgtcctgctggaaatgaccaaggtggaggtcttcagccgctacgaccagggtgccgccgccgaggccctggtgagcgtctacgagcgctccgacaagccggcgaaggacctggccctcaacctgggcctggatcccatcgcgttcgcagccctgcagggcaccgagccggatctgaccgtgctcggtgactgggtgcgccgcctggcgaagttctcgccggactcgcgcgccgtcacgatcgacgcgaacatctaccacaacgccggtgccggcgacgtggcagagctcgcctgggcactggccaccggcgcggagtacgtgcgcgccctggtcgagcagggcttcaccgccaccgaggccttcgacacgatcaacttccgtgtcaccgccacccacgaccagttcctcacgatcgcccgtcttcgcgccctgcgcgaggcatgggcccgcatcggcgaggtcttcggcgtggacgaggacaagcgcggcgcccgccagaatgcgatcaccagctggcgtgacgtcacgcgcgaagacccctatgtcaacatccttcgcggttcgattgccaccttctccgcctccgttggtggggccgagtcgatcacgacgctgcccttcacccaggccctcggcctgccggaggacgacttcccgctgcgcatcgcgcgcaacacgggcatcgtgctcgccgaagaggtgaacatcggccgcgtcaacgacccggccggtggctcctactacgtcgagtcgctcacccgcagcctggccgacgccgcctggaaggaattccaggaggtcgagaagctcggtggcatgtcgaaggccgtcatgaccgagcacgtcaccaaggtgctcgacgcctgcaatgccgagcgcgccaagcgcctggccaaccgcaagcagccgatcaccgcggtcagcgagttcccgatgatcggggcccgcagcatcgagaccaagccgttccccgccgctccggcgcgcaagggcctggcctggcatcgcgactccgaggtgttcgagcagctgatggatcgctccaccagcgtctccgagcgccccaaggtgttcctggcctgcttgggcacccgtcgcgacttcggtggccgcgagggcttctcgagcccggtgtggcacatcgccggcatcgacaccccgcaggtcgaaggcggcaccaccgccgagatcgtcgaggcattcaagaagtcgggcgcccaggtggccgacctctgctcgtccgccaaggtctacgcgcagcagggacttgaggtcgccaaggcactcaaggccgccggcgcaaaggccctgtacctgtcgggcgccttcaaggagttcggtgatgacgccgccgaggccgagaagctgatcgacggacgcctgtttatgggcatggatgtcgtcgacaccctgtcctccacccttgatatcttgggagtcgcgaagtga

SEQ ID NO: 74 - protein, 638 aa, Propionibacterium freudenreichii shermanii
MSSTDQGTNPADTDDLTPTTLSLAGDFPKATEEQWEREVEKVLNRGRPPEKQLTFAECLKRLTVHTVDGIDIVPMYRPKDAPKKLGYPGVAPFTRGTTVRNGDMDAWDVRALHEDPDEKFTRKAILEGLERGVTSLLLRVDPDAIAPEHLDEVLSDVLLEMTKVEVFSRYDQGAAAEALVSVYERSDKPAKDLALNLGLDPIAFAALQGTEPDLTVLGDWVRRLAKFSPDSRAVTIDANIYHNAGAGDVAELAWALATGAEYVRALVEQGFTATEAFDTINFRVTATHDQFLTIARLRALREAWARIGEVFGVDEDKRGARQNAITSWRDVTREDPYVNILRGSIATFSASVGGAESITTLPFTQALGLPEDDFPLRIARNTGIVLAEEVNIGRVNDPAGGSYYVESLTRSLADAAWKEFQEVEKLGGMSKAVMTEHVTKVLDACNAERAKRLANRKQPITAVSEFPMIGARSIETKPFPAAPARKGLAWHRDSEVFEQLMDRSTSVSERPKVFLACLGTRRDFGGREGFSSPVWHIAGIDTPQVEGGTTAEIVEAFKKSGAQVADLCSSAKVYAQQGLEVAKALKAAGAKALYLSGAFKEFGDDAAEAEKLIDGRLFMGMDVVDTLSSTLDILGVAK

SEQ ID NO: 75 - DNA, 2187 bp, Propionibacterium freudenreichii shermanii
gtgagcactctgccccgttttgattcagttgacctcggcaatgccccggttcctgctgatgccgcacgacgcttcgaggaactggccgccaaggccggcaccggagaggcgtgggagacggccgagcagattccggttggcaccctgttcaacgaagacgtctacaaggacatggactggctggacacctacgcaggtatcccgccgttcgtccacggcccgtatgcaaccatgtacgcgttccgtccctggacgattcgccagtacgccggtttctccacggccaaggagtcgaacgccttctaccgccgcaaccttgcggccggccagaagggcctgtcggttgccttcgacctgcccacccaccgtggctacgactcggacaatccccgcgtcgccggtgacgtcggcatggccggtgtggccatcgactccatctatgacatgcgcgagctgttcgccggcattccgctggaccagatgagcgtgtccatgaccatgaacggcgccgtgctgccgatcctggccctctatgtggtgaccgccgaggagcagggcgtcaagcccgagcagctcgccgggacgatccagaacgacatcctcaaggagttcatggttcgtaacacctacatctacccgccgcagccgagtatgcgaatcatctctgagatcttcgcctacacgagtgccaatatgccgaagtggaattcgatttccatttccggctaccacatgcaggaagccggcgccacggccgacatcgagatggcctataccctggccgacggtgttgactacatccgcgccggcgagtcggtgggcctcaatgtcgaccagttcgcgccgcgtctgtccttcttctggggcatcggcatgaacttcttcatggaggttgccaagctgcgtgccgcgcgcatgttgtgggccaagctggtgcatcagttcgggccgaagaacccgaagtcgatgagcctgcgcacccactcgcagacctccggttggtcgctgaccgcccaggacgtctacaacaacgtcgtgcgtacctgcatcgaggccatggccgccacccagggccatacccagtcgctgcacacgaactcgctcgacgaggccatcgccctgccgaccgatttcagcgcccgcatcgcccgtaacacccagctgttcctgcagcaggaatcgggcacgacgcgcgtgatcgacccgtggagcggctcggcatacgtcgaggagctcacctgggacctggcccgcaaggcatggggtcacatccaggaggtcgagaaggtcggcggcatggccaaggccatcgaaaagggcatccccaagatgcgcatcgaggaagccgccgcccgcacccaggcacgcatcgactccggccgccagccgctgatcggcgtgaacaagtaccgcctggagcacgagccgccgctcgatgtgctcaaggtggacaactccacggtgctcgccgagcagaaggccaagctggtcaagctgcgcgccgagcgcgatcccgagaaggtcaaggccgccctcgacaagatcacctgggccgccggcaaccccgacgacaaggatccggatcgcaacctgctgaagctgtgcatcgacgctggccgcgccatggcgacggtcggcgagatgagcgacgcgctcgagaaggtcttcggacgctacaccgcccagattcgcaccatctccggtgtgtactcgaaggaagtgaagaacacgcctgaggttgaggaagcacgcgagctcgttgaggaattcgagcaggccgagggccgtcgtcctcgcatcctgctggccaagatgggccaggacggtcacgaccgtggccagaaggtcatcgccaccgcctatgccgacctcggtttcgacgtcgacgtgggcccgctgttccagaccccggaggagaccgcacgtcaggccgtcgaggccgatgtgcacgtggtgggcgtttcgtcgctcgccggcgggcatctgacgctggttccggccctgcgcaaggagctggacaagctcggacgtcccgacatcctcatcaccgtgggcggcgtgatccctgagcaggacttcgacgagctgcgtaaggacggcgccgtggagatctacacccccggcaccgtcattccggagtcggcgatctcgctggtcaagaaactgcgggcttcgctcgatgcctag

SEQ ID NO: 76 - protein, 728 aa, Propionibacterium freudenreichii shermanii
MSTLPRFDSVDLGNAPVPADAARRFEELAAKAGTGEAWETAEQIPVGTLFNEDVYKDMDWLDTYAGIPPFVHGPYATMYAFRPWTIRQYAGFSTAKESNAFYRRNLAAGQKGLSVAFDLPTHRGYDSDNPRVAGDVGMAGVAIDSIYDMRELFAGIPLDQMSVSMTMNGAVLPILALYVVTAEEQGVKPEQLAGTIQNDILKEFMVRNTYIYPPQPSMRIISEIFAYTSANMPKWNSISISGYHMQEAGATADIEMAYTLADGVDYIRAGESVGLNVDQFAPRLSFFWGIGMNFFMEVAKLRAARMLWAKLVHQFGPKNPKSMSLRTHSQTSGWSLTAQDVYNNVVRTCIEAMAATQGHTQSLHTNSLDEAIALPTDFSARIARNTQLFLQQESGTTRVIDPWSGSAYVEELTWDLARKAWGHIQEVEKVGGMAKAIEKGIPKMRIEEAAARTQARIDSGRQPLIGVNKYRLEHEPPLDVLKVDNSTVLAEQKAKLVKLRAERDPEKVKAALDKITWAAGNPDDKDPDRNLLKLCIDAGRAMATVGEMSDALEKVFGRYTAQIRTISGVYSKEVKNTPEVEEARELVEEFEQAEGRRPRILLAKMGQDGHDRGQKVIATAYADLGFDVDVGPLFQTPEETARQAVEADVHVVGVSSLAGGHLTLVPALRKELDKLGRPDILITVGGVIPEQDFDELRKDGAVEIYTPGTVIPESAISLVKKLRASLDA

SEQ ID NO: 77 - DNA, 1911 bp, Propionibacterium acnes
atgactgatcctgacaacctcgcatccaagtccgtcgaggacaggatgcctgaggagttgagcctggccggggacttcccgaaggtcacccacgagcagtgggaggaggcggtccttaaggttctgaaccggggtcgtcccgagggcaaggaactcaacatcgagcagggaatgaagcgcctcgagccgactacggtcgacggcatccagatcgagccgatgtaccggcgtcaggatgcccccgagaagctcggtgttccgggcgtgccgccgttcactcgtggtaccacgatccgtgaaggcggcatggatgcatgggatgtccgggcccttcatgaggatcccgatgtcgcgttcactaaaaaggccgttatcgccgaccttgagcgtggcgtgacgtccctgtggctgcgggtcggggctgacgccattaagcccgaggacattgcgggtgacctcaaggatgtgttgctcgacctggccaaggttgaggtctctagccgtgatgaccaggaggccgctgctcaggctctccttgatgtttacattgagtccaagatcgatgctgacaagctgtcgtttaacctcggtctggaccccatcggttttgcggcccttaatggcggtaacccagacttgtccgggatggccgagtgggtcaagaagaccgagaattacaagaactcccgccccttcgtcgttgacgccacgatctaccacaacgccggtgctggcgacgtgcacgaactcgcgtgggctgtcgcgaccggcgttgagtacgtccgagctttcattgagcaggggctgacagctgagcaggccttcgattccatcaacttccgcgtcacggccacccacgacgagttcctcactatttcccgtctgcgtgccctacgtaccttgtggaaccgtgttggcgaggtcttcgaggtaccggctgctaagcgtggtgcccgtcaagaagcggtcaccagctggcgcgagctcactcgcgacgatccttacgtcaatatccttcgtggcacgatcgcaaccttcggtgctgctgtcggtggcgctgaggcggtcacgaccttgcctttcgacgccgctattggtttaccgaagagtgacttctctcgtcgtatcgctcgcaacaccggcatcattctcgctgaagagtcgaatattggtcgcgctaatgatccagctggtggctccttctatgtcgaggcactcaccaagaagttggaggatgccggttgggccgagttccaggccgtcgaggctgctggtggcatggctgccgccctcaccggcgaccatgtccgcaccgagctcgacaagctcaacaccgagcgtgccaagcgtcttgccactcgcaagcagccgattacggccgtcagtgagttcccgctgctcgatgccaagtccgtcgagaccaaaccgtatccagcagccccggcccgtgagggtctggaatggcaccgtgacgccgaggtcttcgaggccctcgttgatcgctctgccacttgccctgagcgtccaaaagtgttcctggcctgcctgggaacccgccgtgactttggcccgcgcgagggattctccgccccggtatggcacatcgccggcatggaaacccccgagtgtgaggggggcaccaccgaggaggtcgtgaaggcctttaaggagtctggtgctgatatcgccgacctgtgctcgaacgccaagacctacgcggctcaaggtctcgaggtcgccaaggccctcaaggaggctggcgccaagctggtttacctgtcaggtgccttcaaggaattcggtgatgatgccgccgaggccgagaaggtcatcgacgggcgcatctacctcgggatggacgtcgtggacgtcctgaccgccactctggacacgttgggagttgccaagtga

SEQ ID NO: 78 - protein, 636 aa, Propionibacterium acnes
MTDPDNLASKSVEDRMPEELSLAGDFPKVTHEQWEEAVLKVLNRGRPEGKELNIEQGMKRLEPTTVDGIQIEPMYRRQDAPEKLGVPGVPPFTRGTTIREGGMDAWDVRALHEDPDVAFTKKAVIADLERGVTSLWLRVGADAIKPEDIAGDLKDVLLDLAKVEVSSRDDQEAAAQALLDVYIESKIDADKLSFNLGLDPIGFAALNGGNPDLSGMAEWVKKTENYKNSRPFVVDATIYHNAGAGDVHELAWAVATGVEYVRAFIEQGLTAEQAFDSINFRVTATHDEFLTISRLRALRTLWNRVGEVFEVPAAKRGARQEAVTSWRELTRDDPYVNILRGTIATFGAAVGGAEAVTTLPFDAAIGLPKSDFSRRIARNTGIILAEESNIGRANDPAGGSFYVEALTKKLEDAGWAEFQAVEAAGGMAAALTGDHVRTELDKLNTERAKRLATRKQPITAVSEFPLLDAKSVETKPYPAAPAREGLEWHRDAEVFEALVDRSATCPERPKVFLACLGTRRDFGPREGFSAPVWHIAGMETPECEGGTTEEVVKAFKESGADIADLCSNAKTYAAQGLEVAKALKEAGAKLVYLSGAFKEFGDDAAEAEKVIDGRIYLGMDVVDVLTATLDTLGVAK

SEQ ID NO: 79 - DNA, 2190 bp, Propionibacterium acnes
gtgaccaccctgcctcgtttcgattccattaacctcggggattccccggttcccgcagatgcgcaggagcagttcgccagattggctgcggccgccggtgagcaggagccctggaccactccggagcaaattccggttggtcacctctactccgaggacgtctacggtgacatggactggctagacacctacgccggtctgccaccattcacacatggcccgtacgctaccatgtatgccttccgaccgtggacgattcgccagtacgccggattctccaccgctaaagagtccaacgcgttctaccgtcgtaacctggccgccggtcagaagggcctgtcggtcgccttcgacctgccgacccaccgcggctacgactctgacaacccgcgtgttcccggcgatgtcggtatggcaggcgtggccgtggactccatcctggacatgcgtgagctctttgcgggcatcccgctggaccgcatgtcggtgtccatgacgatgaacggcgcagtgctgccgattcttgccctgtacgttgttaccgctgaggagcaaggcgccaagcccgagcagctcgccggaacgatccagaacgacatcctcaaggagttcatggttcgtaacacctacatctacccgccgctgccttcgatgcggattatctccgacatcttcgcgtacaccagcgcgaacatgccgaagtggaactccatctcgatctccggctaccacatgcaagaagctggcgctaccgccgatatcgagatggcctacactctggccgacggcgttgattacattcgcgccggtgagtcggtgggcctacaggtcgaccagtttgcgccgcgtctttccttcttctgggccattggcaccaacttcttcatggaggtcgccaagatgcgtgcggcccgcatgctgtgggccaagctggtgcaccagttcaacccgaagaatccgaagtcgatgagcttacgcactcactcgcagacctctggctggtcgttgacagcccaggacgtttacaacaacgtgattcgtacttgtgtggaggccatgggagccactcagggacacactcagtccctgcacacgaactccctcgacgaggccatcgctctgccgaccgacttctccgcccgtattgctcgaaacacccagctgttcatccagcaggagtcgggcacctgccgcgttatcgatccttggagcggctcggcctacgtcgagaagctcaccctcgagctggctcgcaaggcctgggctcacatccaggaggtcgagaaggccggtggtatggccaaggccattgagaagggcatcccgaagatgcgcatcgaggaagctgctgctcgtacccaggctcgtatcgactcgggtcgtcagcccctcatcggcgtcaacaagtaccgtctcgacgaggaggagcccctcgaggtcctcaaggtcgacaacactcaggtactcaaggaacagaaggccaaactcgagcagctgcgcgccaaccgcgacgaggaggcgtgccaggccgctctggagaagatcacctgggcagctgccaacccggatccgagtgatcctgaccgtaacctgctcaagctgtgcattgacgctggccgcgcagatgcgtctgtcggtgagatgtctgacgcaatggagaaggtcttcgggcgttacactgcccagatccgtaccattgaaggcgtgtacagcaaggcagccggcaattctgagtccactaagaaggtccacgagctcatcaagcagttcgaggagaaggaaggccgtcgtccgcgtatcatgatcgcgaagatgggccaggacggtcacgaccgtggccagaaggtcgtcgcgaccgcttatgctgaccttggtatggacgttgatgtcggcccgctgttccagaccccggaggagactgctcgacaggctgttgagggtgacgtccacgttgtcggtgtctcctcactggccgccgggcaccttacgctagtgccggccctgcgtaaggaactggacaagcttggtcgctccgacatcatgattgtcgtcggtggcgtcattccgacccaggacttcgacgagctgcgcaaggatggcgcggcggccatctatccgcctggcaccgttatcccggacgctgccgttgagttgatggagaagctgctcgccgcgcacaacgacgactga

SEQ ID NO: 80 - protein, 729 aa, Propionibacterium acnes
MTTLPRFDSINLGDSPVPADAQEQFARLAAAAGEQEPWTTPEQIPVGHLYSEDVYGDMDWLDTYAGLPPFTHGPYATMYAFRPWTIRQYAGFSTAKESNAFYRRNLAAGQKGLSVAFDLPTHRGYDSDNPRVPGDVGMAGVAVDSILDMRELFAGIPLDRMSVSMTMNGAVLPILALYVVTAEEQGAKPEQLAGTIQNDILKEFMVRNTYIYPPLPSMRIISDIFAYTSANMPKWNSISISGYHMQEAGATADIEMAYTLADGVDYIRAGESVGLQVDQFAPRLSFFWAIGTNFFMEVAKMRAARMLWAKLVHQFNPKNPKSMSLRTHSQTSGWSLTAQDVYNNVIRTCVEAMGATQGHTQSLHTNSLDEAIALPTDFSARIARNTQLFIQQESGTCRVIDPWSGSAYVEKLTLELARKAWAHIQEVEKAGGMAKAIEKGIPKMRIEEAAARTQARIDSGRQPLIGVNKYRLDEEEPLEVLKVDNTQVLKEQKAKLEQLRANRDEEACQAALEKITWAAANPDPSDPDRNLLKLCIDAGRADASVGEMSDAMEKVFGRYTAQIRTIEGVYSKAAGNSESTKKVHELIKQFEEKEGRRPRIMIAKMGQDGHDRGQKVVATAYADLGMDVDVGPLFQTPEETARQAVEGDVHVVGVSSLAAGHLTLVPALRKELDKLGRSDIMIVVGGVIPTQDFDELRKDGAAAIYPPGTVIPDAAVELMEKLLAAHNDD

SEQ ID NO: 81 - DNA, 2178 bp, Selenomonas ruminantium lactilytica
atgagtaacgaaaccggtggcgagctggatctgcaggctatcttgaagaaagccgagcagcagacagactttcccgatgtgcctttggacgagtttaccccgcccacttatgaggagtggaaggaagcctgtatcgccctcttgaagggagcgccctttgagaagaaaatgtacaccaaaacctatgagggcattacctttgatcccatgtatttccgcaaggataccgaagacatcctgccgaagaattccttcccgggtatggacgatttcctgcgtggtgcccagcccagcggatatctgggaaaaccctgggggatagcgcaggcctgtgatgaaaccatgcccgcagaaaacaatgagctgctgcgccacgagcaggagaaaggctccaccatctaccatatcaagctggacagtgccagcctgaaggcgcaggatgtgcgcaaggcagctgcccccggggacgagggcgtgtcggtgaccaccctggatgatatgcacaccttgctcaatggcctgaaactcgataaatatcctctttacctctatgctggggaatcggcgctgccgatgctttccctgtttgccggggctttgaaggcctctggtcaggatctaaagcagatccggggcatcgtcggggctgatcccttaggggaattggctgctggcggcaagaatagcaaagatacggcgagcctttacgatgaaatggcccgctgtgccaaatgggccattgcccatgctccggggctcaagactgtgttcgtgcgcagtgatgtctacagccggggcggtgccaacgacgtgcaggagtcggcttataccctggccacggccgtagcctatctgcgggccatgctggaacgggggctctccattgaagaagctgccgggcagatcatgttcggtttctccatgggggccaacttcttcctgcagattgccaagctccgtgccctgcgtccgctctggtcgcagatcgtggaggcctttggtggcagcaaggaagcccagcgcatgcatatccatgcccgtccggccttgttcttcaagaccgtctatgatccctatgtcaatatgcttcggaataccacggaaatcttctccggtgtcgtgggcggtgtggattcctttgagagctcgccctttgatgagcccatccgcaaaggcgacgaattctcccgccgcattgcccggaatgtgcagatcatcctgcaggaggaattcggcctgctacagcccatcgatcccgcaggcggttcctgggctgtggagactttgaccaagcagatgaaggaaaagatctgggctgagttccaggtcatcgagggcaagggcggcatcctgaaagccttgcaggaagggtatccgcaaagtgagattgcgggcgttctggcggcccgtttcaaggcttcggaaacccgcaaggacaggatcgtaggcaacaatatgtatccgaatatgacggaaaccctgctggatccgcgtcccgaagatatggctgagaataagaaacagcgcacggctcaggtagaggaatatctggctgatattgatgaagccttcaagcttgaaatgctgacagccctgaaggctggcaaggacgaaggggaactggccattgctgcggctctggctggcgccactacggaagagattgccggtgctctggctggcggagtgagcgaagaagtcgcgtccattgccccccatcgctggagcgagcgctttgaagccctgcggaagctgacggaggattacaaggcagaacatcacgacaatgtcaagatcttcctggccaatatggggcccatcccacagcacaaggccagagcggatttcaccacgggcttcctgcaggtaggtgcctttgaggtgctgaccaataacggcttccccacggtggaggaagcagcccaggcagccaaggaatccggggcggatgccgtggttatctgctccacggatgcgacttatcccgaaatcgtgccggagctggcaccgaaacttcatgaggtcctgcccaatgccacggtattcttggccggagcggcaccgaaagacctgcttgaaacctacaatgaagcgggcattgatgagtatatctccgtcaaggccaactgctataagatcctgcagctcctgcagcagaagaaagggatgattgcataa

SEQ ID NO: 82 - protein, 725 aa, Selenomonas ruminantium lactilytica
MSNETGGELDLQAILKKAEQQTDFPDVPLDEFTPPTYEEWKEACIALLKGAPFEKKMYTKTYEGITFDPMYFRKDTEDILPKNSFPGMDDFLRGAQPSGYLGKPWGIAQACDETMPAENNELLRHEQEKGSTIYHIKLDSASLKAQDVRKAAAPGDEGVSVTTLDDMHTLLNGLKLDKYPLYLYAGESALPMLSLFAGALKASGQDLKQIRGIVGADPLGELAAGGKNSKDTASLYDEMARCAKWAIAHAPGLKTVFVRSDVYSRGGANDVQESAYTLATAVAYLRAMLERGLSIEEAAGQIMFGFSMGANFFLQIAKLRALRPLWSQIVEAFGGSKEAQRMHIHARPALFFKTVYDPYVNMLRNTTEIFSGVVGGVDSFESSPFDEPIRKGDEFSRRIARNVQIILQEEFGLLQPIDPAGGSWAVETLTKQMKEKIWAEFQVIEGKGGILKALQEGYPQSEIAGVLAARFKASETRKDRIVGNNMYPNMTETLLDPRPEDMAENKKQRTAQVEEYLADIDEAFKLEMLTALKAGKDEGELAIAAALAGATTEEIAGALAGGVSEEVASIAPHRWSERFEALRKLTEDYKAEHHDNVKIFLANMGPIPQHKARADFTTGFLQVGAFEVLTNNGFPTVEEAAQAAKESGADAVVICSTDATYPEIVPELAPKLHEVLPNATVFLAGAAPKDLLETYNEAGIDEYISVKANCYKILQLLQQKKGMIA

SEQ ID NO: 83 - DNA, 2202 bp, Selenomonas ruminantium lactilytica
atgggaagctttacgaaccctgactttaccaatatgagcctgcgggaacctacgggggctgatgtcaaagagtgggaaaaactcttcagcgcacaggccggtgcagatttcgacgcttatacccgccgcaccatggaacacattccggtaaagcctttatataaccatgacgaatatgaccatatgaaccatctggattttgccagcggcattccgccctgcctgcgcgggccgtactccaccatgtacgtcttccgtccctggaccgtgcgtcagtacgccggtttctccacggcagaggaatccaacgcattctatcggcgcaatctggcagccggtcagaaaggtctgtccattgcctttgacctgccgacgcaccgcggctacgatgcggataacccccgtgttgtaggtgatgtgggtaaggctggcgtatccgtgtgctccatgctggatatgaatatcctgttctcggggattcctctcgatcagatgtccgtatccatgaccatgaacggcgccgtgctgccgattctggccttctttatccagtccggcgtggagcagggtgtggataagaagatcatggcgggcaccatccagaacgatatcctgaaagagttcatggtgcgtaacacctatatctatccgccggaaatgtccatgcgcatcatcggtgatatcttcgaatacaccacgaaatacatgccgaagttcaacagcatctcaatttccggttaccatatgcaggaggccggtgctccggccgatatcgagctgggctataccctggcagatggtctggaatacatccgcacgggtatcaatgccggcctgccggtggatgcctttgccaagcgtctgtccttcttctgggccatcggcaagaactacttcatggaagtggccaagatgcgggcggcgcgcgtgctctgggcgaagatcgtcaaatcctttggcgctcaggaacccaagtccatggcacttcgtacccattgtcagacttcgggttggtcccttacggcacaggatcccttcaacaatatctcccgcacggctatggaggccatgggcgcggccctgggccatacccagtccctgcataccaatgccctggacgaggccattgccctgccgacggacttctcggcccgcattgcccgcaatacgcagctctatatccaggacgagaccaaggtctgcaagatcatcgatccctggggcggctcctattatgtggaggcccttaccaacgagatcattcgccgtgcctgggcccatattcaggaagtcgaggccttgggcggcatggccaaggctatctccacgggtttgcccaagatgcgtatcgaggaagcggcagcccgccgtcaggctcagatcgactcgggcaacgagaccattgtgggcttgaacaaataccggctggagaaggaagatcccctggagatcctggccatcgacaatactgccgtgcgcaatgcccaggtggaacgcctcgaaaaactgcggcgtgaacgcaatgaggacgatgtgcgccgggcccttgaggccatcacgaaagcagccgatagccgtgataacggcaatctgctggaatgcgctgtggaagctgctcgggtgcgtgcgtctttgggcgagatttccgacgcagtggaaaaggtttctggccgctatcaggcggttattcataccatttcgggagtctattcttccgagttcacagacaagaccgagctggataaagcccgggctatggccgacgagttcgaagaactcacaggccgccgtccccgcatctttgtagccaagatgggacaggacggtcacgaccgcggtcagaaagttattgcttcgtccttcgccgatatgggctgggacgttgatgtgggcccgctgttccagacaccggaggaaacggcacaggatgctgtggataacgatgtgcatatggtgggcttcagttcactggcggcggggcataacaccctcctgccccagctggtggatgaactcaaaaaactgggccgtgaggatatcatggtctgcatcggcggtgtcatccccgtgcaggattatgacaacctctataaacacggcgcagtagctatctttgccccaggaaccaatattcctgaggctggtataaagcttttgaacctgctgcttgaccgggcgaaagaagaactggccgaggagtaa

SEQ ID NO: 84 - protein, 733 aa, Selenomonas ruminantium lactilytica
MGSFTNPDFTNMSLREPTGADVKEWEKLFSAQAGADFDAYTRRTMEHIPVKPLYNHDEYDHMNHLDFASGIPPCLRGPYSTMYVFRPWTVRQYAGFSTAEESNAFYRRNLAAGQKGLSIAFDLPTHRGYDADNPRVVGDVGKAGVSVCSMLDMNILFSGIPLDQMSVSMTMNGAVLPILAFFIQSGVEQGVDKKIMAGTIQNDILKEFMVRNTYIYPPEMSMRIIGDIFEYTTKYMPKFNSISISGYHMQEAGAPADIELGYTLADGLEYIRTGINAGLPVDAFAKRLSFFWAIGKNYFMEVAKMRAARVLWAKIVKSFGAQEPKSMALRTHCQTSGWSLTAQDPFNNISRTAMEAMGAALGHTQSLHTNALDEAIALPTDFSARIARNTQLYIQDETKVCKIIDPWGGSYYVEALTNEIIRRAWAHIQEVEALGGMAKAISTGLPKMRIEEAAARRQAQIDSGNETIVGLNKYRLEKEDPLEILAIDNTAVRNAQVERLEKLRRERNEDDVRRALEAITKAADSRDNGNLLECAVEAARVRASLGEISDAVEKVSGRYQAVIHTISGVYSSEFTDKTELDKARAMADEFEELTGRRPRIFVAKMGQDGHDRGQKVIASSFADMGWDVDVGPLFQTPEETAQDAVDNDVHMVGFSSLAAGHNTLLPQLVDELKKLGREDIMVCIGGVIPVQDYDNLYKHGAVAIFAPGTNIPEAGIKLLNLLLDRAKEELAEE

SEQ ID NO: 85 - DNA, 462 bp, Propionibacterium acidi-propionici
atggagaacttcaacaacgatcctttcgcgtgtatcgatcacgtcggctacgcggtcaaggacatggacgaggccatcaagtatcacaccgaggtgctcggcttccacgtgctgctgcgtgagaagaacgagggtcacggcgtcgaggaggcgatgatcgccaccggcaagcgcggcgaggagagcaccgtcgtccagctgctcgcccccctcggcgaggacaccaccatcggcaagtacctggccaagaacaagaacatgatccagcaggtgtgctaccgcacctacgacatcgacaagaccatcgcgaccctcaaggagcgcggggccaggttcacctccgaggagccctcctccggcaccgccgggtcccgggtcatcttcctccacccgaagtacaccggcggtctgctcatcgagatcaccgagcccccggccggcggcatgccctacaaggactga

SEQ ID NO: 86 - protein, 153 aa, Propionibacterium acidi-propionici
MENFNNDPFACIDHVGYAVKDMDEAIKYHTEVLGFHVLLREKNEGHGVEEAMIATGKRGEESTVVQLLAPLGEDTTIGKYLAKNKNMIQQVCYRTYDIDKTIATLKERGARFTSEEPSSGTAGSRVIFLHPKYTGGLLIEITEPPAGGMPYKD

SEQ ID NO: 87 - DNA, 447 bp, Propionibacterium freudenreichii shermanii
atgagtaatgaggatcttttcatctgtatcgatcacgtggcatatgcgtgccccgacgccgacgaggcttccaagtactaccaggagaccttcggctggcatgagctccaccgcgaggagaacccggagcagggagtcgtcgagatcatgatggccccggctgcgaagctgaccgagcacatgacccaggttcaggtcatggccccgctcaacgacgagtcgaccgttgccaagtggcttgccaagcacaatggtcgcgccggactgcaccacatggcatggcgtgtcgatgacatcgacgccgtcagcgccaccctgcgcgagcgcggcgtgcagctgctgtacgacgagcccaagctcggcaccggcggaaaccgcatcaacttcatgcatcccaagtcgggcaagggcgtgctcatcgagctcacccagtacccgaagaactga

SEQ ID NO: 88 - protein, 148 aa, Propionibacterium freudenreichii shermanii
MSNEDLFICIDHVAYACPDADEASKYYQETFGWHELHREENPEQGVVEIMMAPAAKLTEHMTQVQVMAPLNDESTVAKWLAKHNGRAGLHHMAWRVDDIDAVSATLRERGVQLLYDEPKLGTGGNRINFMHPKSGKGVLIELTQYPKN

SEQ ID NO: 89 - DNA, 459 bp, Propionibacterium acnes
atggagaacttcaacaacgatccttttgcgtgtattgatcacgtcggttttgccgtcaaggacatggacgaggccatcaagtaccactgcgacgtgctgggcttccgggtgctcttccgtgagaagaacgagggacatggcgtcgaggaggccatgctcggtaccggcaagcgcggcgaggagtcgaccgtcgttcagttgctcgccccgctcagcgaagacagcaccattggaaagtacatttccaagaataagaatatgatccagcaggtgtgctaccgcacctacaacctggacaagacgatcgccaccctcaaggagcgtggcgccgtcttcaccggcgagccctccatcggaaccgctggctcccgtgtcatcttccttcaccccaagtacaccggcggtctcctcatcgagatcaccgagcccccggtcggcggcatgccttacaaggactga

SEQ ID NO: 90 - protein, 152 aa, Propionibacterium acnes
MENFNNDPFACIDHVGFAVKDMDEAIKYHCDVLGFRVLFREKNEGHGVEEAMLGTGKRGEESTVVQLLAPLSEDSTIGKYISKNKNMIQQVCYRTYNLDKTIATLKERGAVFTGEPSIGTAGSRVIFLHPKYTGGLLIEITEPPVGGMPYKD

SEQ ID NO: 91 - DNA, 417 bp, Selenomonas ruminantium lactilytica
atgtttaaggtattaggtgtagaccatattggtattgctgttggtgacctcaaggaagttggctccttctggggcgatatgctgggcctgcctaacaacggtgaggaaaccgttgaagagcagaaggtgaccacgggtttcttcccgacgcccaatggcagcgagatcgaactgctggcagctacggcggatgattccccgattgccaagttcatcgaaaagaacggcggccgtggcggcatccagcacattgccctgcgtgtggacaacctcgaagcagctctggcggacctcaaggaaaaaggcgtgcgtctgattgacgaaaagccccgcaagggtgccggcggcgccaagattgccttcgttcatccgaaagcttctcatggcgtgctgctcgaactttgccagcgtgactga

SEQ ID NO: 92 - protein, 138 aa, Selenomonas ruminantium lactilytica
MFKVLGVDHIGIAVGDLKEVGSFWGDMLGLPNNGEETVEEQKVTTGFFPTPNGSEIELLAATADDSPIAKFIEKNGGRGGIQHIALRVDNLEAALADLKEKGVRLIDEKPRKGAGGAKIAFVHPKASHGVLLELCQRD

SEQ ID NO: 93 - DNA, 1506 bp, Mus musculus
atgtcttcacctgcacaacctgcagtccctgccccgctggccaacttgaagattcaacataccaagatctttataaacaatgaatggcatgattcggtgagcagcaagaaatttcctgtccttaaccctgcaactgaggaggtcatctgccacgtggaagaaggggacaaggctgatgttgacaaagctgtgaaggccgcaagacaggctttccagattggctccccatggcgcaccatggatgcttcagagaggggccgcctgctgaacaagctggctgacttaatggagagagatcgtctgctgctggctacaatggaatcgatgaatgctgggaaagtctttgctcatgcatacctgttggatgtagagatcagcataaaagcattacagtacttcgcaggctgggctgacaagatccatggccaaacaatacccagtgatggaaacatattcacttatacaaggcgtgaacctattggggtgtgtggccaaatcatcccttggaatggtccattgattatattcacttggaagttaggccctgcccttagctgtgggaacactgtggttgtcaagccagcagagcaaactcctctcacagctcttcacatggcatctttaataaaagaggcagggtttcctcctggcgtggtgaacattgtccctggttatgggccaactgcagggggagccatctcctcccacatggacatcgacaaagtgtccttcacaggatcaacagaggttggcaaattaatcaaggaagctgcagggaaaagcaatctgaagagagtcaccctggagctggggggaaagagcccttgcattgtgtttgcagatgccgacttggacagtgctgttgagtttgcacaccaaggagtgttcttccaccagggtcagatttgtgttgcagcgtccaggctttttgttgaggaatcaatttacgatgagtttgtgaggaggagtgtggagcgggctaagaaatacattctaggaaatcctctgaactccggaataaatcaaggtcctcagattgacaaggaacaacacaataaaatacttggtctcattgagagtgggaagaaagaaggagccaagctggagtgtggtggaggtcgctgggggaacaaaggcttctttgtccaacccacagtcttctccaatgtgactgatgagatgcgcattgccaaagaggagatatttggaccagtgcaacaaatcatgaagtttaaatctatggatgatgtgatcaagagagcaaacaacactacctatggtttagcagcaggagtcttcactaaagacctggataaggccatcactgtgtcatctgctctgcaggctgggatggtgtgggtgaactgctatttggctgtccctgtccaatgcccatttggtggattcaagatgtctggaaatgggcgagaactgggcgaacatggtctttatgaatacactgagctcaagacagttgcaatgcaaatatctcagaagaactcataa

SEQ ID NO: 94 - protein, 501 aa, Mus musculus
MSSPAQPAVPAPLANLKIQHTKIFINNEWHDSVSSKKFPVLNPATEEVICHVEEGDKADVDKAVKAARQAFQIGSPWRTMDASERGRLLNKLADLMERDRLLLATMESMNAGKVFAHAYLLDVEISIKALQYFAGWADKIHGQTIPSDGNIFTYTRREPIGVCGQIIPWNGPLIIFTWKLGPALSCGNTVVVKPAEQTPLTALHMASLIKEAGFPPGVVNIVPGYGPTAGGAISSHMDIDKVSFTGSTEVGKLIKEAAGKSNLKRVTLELGGKSPCIVFADADLDSAVEFAHQGVFFHQGQICVAASRLFVEESIYDEFVRRSVERAKKYILGNPLNSGINQGPQIDKEQHNKILGLIESGKKEGAKLECGGGRWGNKGFFVQPTVFSNVTDEMRIAKEEIFGPVQQIMKFKSMDDVIKRANNTTYGLAAGVFTKDLDKAITVSSALQAGMVWVNCYLAVPVQCPFGGFKMSGNGRELGEHGLYEYTELKTVAMQISQKNS

SEQ ID NO: 95 - DNA, 1371 bp, Clostridium botulinum
atggagaatataagaaatatattggaaaaacagaagagtttttttgataagggatatacaaaggatattaactttagaatagaggctttaaaaaaattaaaacataatataaaaatcaatgaaaacaatatatttaaagctttaaagatagatttaaataaatcagaatttgaaacatttataacagaaataggtattgtatatgatgaaataaatggagcgattaaaaatataaaaaaatggtcaaagcctaaaaaagtaaaaactccaattactaattttttagctagtagttatatatacaatgagccttacggtgtagctttaataatgtctccctggaattatccatttcaacttattatggctcctttagtaggagctataagtgctggtaattgtgttttgttaaagccctccgaattagcaatagaaacggaaaaaataatagttaaaataataaaagatacattttctgatgaatatataggggttatcactggaggaatagaagagagtacggctttgcttaaagaaaagtttgactatatattttatacgggtggaataaatgtaggtaaaatagttatgagagcagctgcagagcatttaacccccataaccttagaattaggagggaaaagtccctgcattgttgacaaggatgctaacatagatttggctgccagaagaatagcttggggaaaatttttaaatgctggacaaacctgtgtagcaccagattatttagtagtgcatagaaatataaaagaaaaattaataagttcaatagaaaattatataattgagttttttggagaaaatacctttgaaagtgaagattatcctagaataataaatgaaagacactttaaaagattagaaggatatttaaaggaaggaaaaatagtttctggaggaaatacagatataaataatttatatatagaaccaactattatagaaggaataaattttgaaaatagaataatggaggaagaaatatttggccctgttttcccagttatagaatttgaaaacatagataaagttatagaaatagtaaaaaataatcctaaaccactagcactatattatttttctgagaataaagagaaacaggaatttattattaaaaatatatcctttggcgggggatgtataaatgatactataatgcatttgtctacttctacattaccctttggtggtgtaggtaatagtgggataggaggctatcatggcagagcaagttttgacacattttctcataaaaaaagtatacttaaaaagagtaatttaatagatgtaaaaataagatatgcaccttttaaaggaaaaataaatttagcaaaaagattatttaagtaa

SEQ ID NO: 96 - protein, 456 aa, Clostridium botulinum
MENIRNILEKQKSFFDKGYTKDINFRIEALKKLKHNIKINENNIFKALKIDLNKSEFETFITEIGIVYDEINGAIKNIKKWSKPKKVKTPITNFLASSYIYNEPYGVALIMSPWNYPFQLIMAPLVGAISAGNCVLLKPSELAIETEKIIVKIIKDTFSDEYIGVITGGIEESTALLKEKFDYIFYTGGINVGKIVMRAAAEHLTPITLELGGKSPCIVDKDANIDLAARRIAWGKFLNAGQTCVAPDYLVVHRNIKEKLISSIENYIIEFFGENTFESEDYPRIINERHFKRLEGYLKEGKIVSGGNTDINNLYIEPTIIEGINFENRIMEEEIFGPVFPVIEFENIDKVIEIVKNNPKPLALYYFSENKEKQEFIIKNISFGGGCINDTIMHLSTSTLPFGGVGNSGIGGYHGRASFDTFSHKKSILKKSNLIDVKIRYAPFKGKINLAKRLFK

SEQ ID NO: 97 - DNA, 1503 bp, Saccharomyces cerevisiae
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 98 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 99 - DNA, 1449 bp, Rhodococcus opacus
atgaccgactacgacaagctctacatcggcggcaaatgggtggccccggccaccgaccaggtgctcgacgtgttctccccggccaccgaggaacgcgtcggccgctgtccggtcgcgtcgcctgccgacatcgacgacgccgtcgccgtcgcccggcgggcgttcgacgagggtccgtggccgcagacgacgcccgccgagcgcggggagatcctcgcgaaggcggcgaagctcatcgaggagcgcggcgagaccctcaacgcgctgatctcgtcggagatgggtcagccgcccgcgatggtcgggatgatgcagcagacgccgtcgctcgcgaccctgaatttctatgccggcctcgcgaacgacttcgagtgggagcagacccgcaccggcgtgttcggtcagacgaaggtgctgcgggagccggtcggcgtggtggccgcggtcctcgcctggaacgtgccgctgttcctcgccgtcaacaaactgtccccggcgctgctcgccgggtgcaccgtgctgctgaaaccggcacccgaatccccgctctccacccacgttctcgccgagatcttcgccgaggccggggtccccgagggcgtcatctccgttctgcccggcggcgccgagaccggcgaatacctggtgtcgcatcccgggatcgacaagatcacgttcaccggcagcagtcccgtcggccgcaagatcggggccatcgccgcgcagaacctcaagcgctgctccctcgaactcggcggcaaatcggcggccatcatcctcgaggacgccgacctcgcgtccacgatgccgatgctggtgatgtccgggctgatgaacaccggtcaggcgtgtgtcgcgcagacccggatcctcgccccgcggtcccgctacgacgaggtgctggacgccctcgtcgcgggtgccggattcatggccgtcggcgacccgtccgacccggccgcgcaactcgggccgctcatctccgagaagcagcgcgaccgcgtcgagggttacatcgccaagggcagggaacagggcgcgcgcgtggtgctcggcggcggccgcccggcgggcctcgacaagggctggtacgtggagccgaccatctttgccgacgtcgacaactcgatgaccatcgcccgcgaggagatcttcgggcccgtactgtccgtgatcccctacgattccgaggacgaggcgatcaagatcgccaacgactccgactacggcctcgccggctcggtgtacaccaccgacatcgagcacggtctcgcggtcgcgaagcagatccgcaccggcacgtacgccatcaactggtacgcattcgatccgggatcaccgttcggcgggtacaaggcctccggcatcggccgcgagaacggaccggaggggctcgaagcgttctgcgagaccaagtccgtcctcatgccgcccggctacgcggggtag

SEQ ID NO: 100 - protein, 482 aa, Rhodococcus opacus
MTDYDKLYIGGKWVAPATDQVLDVFSPATEERVGRCPVASPADIDDAVAVARRAFDEGPWPQTTPAERGEILAKAAKLIEERGETLNALISSEMGQPPAMVGMMQQTPSLATLNFYAGLANDFEWEQTRTGVFGQTKVLREPVGVVAAVLAWNVPLFLAVNKLSPALLAGCTVLLKPAPESPLSTHVLAEIFAEAGVPEGVISVLPGGAETGEYLVSHPGIDKITFTGSSPVGRKIGAIAAQNLKRCSLELGGKSAAIILEDADLASTMPMLVMSGLMNTGQACVAQTRILAPRSRYDEVLDALVAGAGFMAVGDPSDPAAQLGPLISEKQRDRVEGYIAKGREQGARVVLGGGRPAGLDKGWYVEPTIFADVDNSMTIAREEIFGPVLSVIPYDSEDEAIKIANDSDYGLAGSVYTTDIEHGLAVAKQIRTGTYAINWYAFDPGSPFGGYKASGIGRENGPEGLEAFCETKSVLMPPGYAG

SEQ ID NO: 101 - DNA, 1590 bp, Entamoeba dispar
atggacgtttatgtactatcattgtcagatgttcttcttaatattttactcattggggttagtattttaggtgttttattcctcatttttcaaggtttaaagtatattattggtgattctatggaaaagaaattgtttgataaaagattagaacaaattaaaaaccaacaacccttagaaccaacaaagtaccaggacattcaaattatttgtaaaacacttaaagaatcatattctacaaatgcattaagacctttagatgctaggatggaagtattatattgtttatatagaatggtagttgataataaacaagcattaagtaatgctattagagaagatcttcatagagatgttggtatgtgtgttgctgaagtaaattctgttattcatgaaatcaattttttaaggaagaacttaaaaaaatatcttagaagaaaacaagtcccaactgtttgtgctcaactctttggaaaatcgtttgttgctcgtgagccttacggttgtgtatgtattatttctccatggaatttccctgctaacttatcattaattccatgtgctggagctattgcatgtggaaatacagtatttttaaaaatgagtaaatactctatggcaacttctaaacttattgcagaattatgtgataaatatattccatcagagtatttgcgttgtgaatatttaactggaagagaagctattcaagaatgttgttctgctccatttgattattacttctttacaggatctacttatgttggtaaacttgttaaccaagctgctgcagagaaaatggttcctgctacattagaattaggaggaaagaatcctgctattgttgataaaaatgttaatttaaaagttgctgctaagagaatagcttgggcaaagtcaattaatgccggacaaatatgtgtttgtgttgatcatgtttttgttccacgaagtattaagaaagaattttgtgaagctgtaaagaatagtttcatcaaattctttggagaagatcaaaagaaaagtgaagattttggtagaataataactaaaagtgcagcaaaaaaaatgaaagaaattatcgatcaaagtgatgtttattatggaggagaagttgatatagaaaataaatatgttcaaccaactattcttcaaaatgttaaaattgatgatctctgtatgaaagaagaaatttttggtccaattctcccagttattgaatatgatactcttgatgaagtatttgaaatggttaaacaacatccaaatccattggcatgttatgtttttacagaagataataatatgtttgaacgtgttatagcaaaaattaattcaggtgctatctataacaatgatagtattgttcatttattaaatccaaatttaccttttggaggaaattgtcaaagtggtattggctgttatcatggaaaatatacatttgatacattctctcgtccacgtgctgtttgtaatggtcatactagattagatttatcattgaaagattggccatttacttcattccaatcatgggcagtagaccgtatggctgcaagtgaaattccagttgtttcatatctttaa

SEQ ID NO: 102 - protein, 529 aa, Entamoeba dispar
MDVYVLSLSDVLLNILLIGVSILGVLFLIFQGLKYIIGDSMEKKLFDKRLEQIKNQQPLEPTKYQDIQIICKTLKESYSTNALRPLDARMEVLYCLYRMVVDNKQALSNAIREDLHRDVGMCVAEVNSVIHEINFLRKNLKKYLRRKQVPTVCAQLFGKSFVAREPYGCVCIISPWNFPANLSLIPCAGAIACGNTVFLKMSKYSMATSKLIAELCDKYIPSEYLRCEYLTGREAIQECCSAPFDYYFFTGSTYVGKLVNQAAAEKMVPATLELGGKNPAIVDKNVNLKVAAKRIAWAKSINAGQICVCVDHVFVPRSIKKEFCEAVKNSFIKFFGEDQKKSEDFGRIITKSAAKKMKEIIDQSDVYYGGEVDIENKYVQPTILQNVKIDDLCMKEEIFGPILPVIEYDTLDEVFEMVKQHPNPLACYVFTEDNNMFERVIAKINSGAIYNNDSIVHLLNPNLPFGGNCQSGIGCYHGKYTFDTFSRPRAVCNGHTRLDLSLKDWPFTSFQSWAVDRMAASEIPVVSYL

SEQ ID NO: 103 - DNA, 972 bp, Aspergillus niger
atgtctttaccatcgcacttcaccatcaacaccggggccaaaatccctgctgtgggattcggcacctggcaagcgaagccccttgaggtagaaaacgccgtcgaagtggccctcagggagggttaccgccacattgattgcgctgccatctatcgcaatgagaccgaagtcggcaatggtattcgcaagtccggggtgccccgcgaagagatcttcatcactggcaagctgtggaacaccaagcacgccccggaggacgtggagccagccctcgacaagaccctgcaagatctgggcgtcgcctacctggatctctatctcatgcactggccctgtgccttcaagggtggcgacaaatggtttcctctcaacgacgatggagtgttcgacttggccaacatcgactacatcaccacctaccgcgccatggagaaattgctagcgaccggcaaggtacgcgccattggcgtgtccaacttcaacatccgccggttggaagagctgctcggccaagtttccgttgtgcccgccgtcaaccagattgaagcccatccttacctgcaacagccggacctgctgcaattctgccagagcaaaggcattctcatcgaggcctactcgccgctcggcaataaccagactggtgagccgcgcaccgtcgacgatccactcgtgcaccgcgtggcgggcgagctaagcttggaccccggaccactgttggccagctggggggtgcagcgtggcactgtcgtgctatccaagagtgttacccccgctcgaattgcagctaatttgcgagtccgggcattgccagagggggcttttgcgcagttgaactcgctggaacgccacaaacgcttcaacttcccggggcattggggctatgacatctttgaggaagtcggtgaggaggcggtgcgccagactgcactggctgcaggccccagtaacaaggttaagtttactgtatag

SEQ ID NO: 104 - protein, 323 aa, Aspergillus niger
MSLPSHFTINTGAKIPAVGFGTWQAKPLEVENAVEVALREGYRHIDCAAIYRNETEVGNGIRKSGVPREEIFITGKLWNTKHAPEDVEPALDKTLQDLGVAYLDLYLMHWPCAFKGGDKWFPLNDDGVFDLANIDYITTYRAMEKLLATGKVRAIGVSNFNIRRLEELLGQVSVVPAVNQIEAHPYLQQPDLLQFCQSKGILIEAYSPLGNNQTGEPRTVDDPLVHRVAGELSLDPGPLLASWGVQRGTVVLSKSVTPARIAANLRVRALPEGAFAQLNSLERHKRFNFPGHWGYDIFEEVGEEAVRQTALAAGPSNKVKFTV

SEQ ID NO: 105 - DNA, 1020 bp, Streptococcus pneumoniae
atgaaagctgttgttgtaaatccagaaagcactggtgttgctattgaagaaaaagtactccgtccacttgaaactggggaagcacttgtagaagttgaatactgtggcgtttgccacaccgacctccacgttgctcatggtgactttggtcaagtcccaggacgtgttcttgggcacgaaggtatcggtatcgttaaagagattgctccagatgtgaaaagccttaaagtcggtgaccgcgtcagcgttgcttggttctttgaaggatgtggcacttgcgaatactgtacaactggtcgcgaaaccctttgccgtacagtgaaaaatgctggctactcagtagacggtggtatggctgaacaatgtatcgcaactgctgactatgctgtcaaagttcctgacggacttgatccagcccaagcttcttctatcacatgtgctggagtaacaacctataaagctatcaaagaagcaaaagttgaaccaggccaatgggttgttctttacggtgctggtggacttggtaacctcgctgttcaatacgctaaaaaagtattcaatgctcatgttatcgcagtcgatatcaacaatgacaaacttacccttgcaaaagaagtaggcgctgacattgtgattaacggcctcgaagttgaagatgtagctggactcattaaagaaaaaactgatggaggagctcattcagctgtcgtaactgctgtgtctaaagttgccttcaaccaggctgttgactccattcgtgctggtggtcgcgtcgtcgctgttggtcttccttctgaaatgatggaactcagcatcgttaaaacagtcctcgatggaatccaagtcatcggttctcttgtcggaactcgtaaagacttagaagaagccttccaatttggtgcagaaggtctggtagtcccagttgttcaaaaacgtccagtagaagatgctgttgccattttcgacgaaatggaaaaaggccaaatccaaggacgtatggtactcgacttcacccactaa

SEQ ID NO: 106 - protein, 339 aa, Streptococcus pneumoniae
MKAVVVNPESTGVAIEEKVLRPLETGEALVEVEYCGVCHTDLHVAHGDFGQVPGRVLGHEGIGIVKEIAPDVKSLKVGDRVSVAWFFEGCGTCEYCTTGRETLCRTVKNAGYSVDGGMAEQCIATADYAVKVPDGLDPAQASSITCAGVTTYKAIKEAKVEPGQWVVLYGAGGLGNLAVQYAKKVFNAHVIAVDINNDKLTLAKEVGADIVINGLEVEDVAGLIKEKTDGGAHSAVVTAVSKVAFNQAVDSIRAGGRVVAVGLPSEMMELSIVKTVLDGIQVIGSLVGTRKDLEEAFQFGAEGLVVPVVQKRPVEDAVAIFDEMEKGQIQGRMVLDFTH

SEQ ID NO: 107 - DNA, 1089 bp, Salmonella enterica
atgaaccacactgaaatccgcgtcgttaccggcccggcgaattatttttcccatgctggaagcctcggaagactgacagactttttcacgccggaacagctttcccacgccgtttgggtgttcggcgaacgcgcgattgccgccgcccggccttacctgccggaagcgtttgaacgtgctggcgcaaaacatctgcagtttaccggccattgtagcgaacgccatgttgcccaactggcgcacgcctgcaacgacgatcgtcaggtggtgataggcgtcggcggcggcgcgctgctcgataccgccaaagcgctcgcccgccgtctggcgctgccgtttgtcgctatcccgaccatcgcggcgacctgcgccgcctggacaccgctttccgtctggtataacgacgcgggacaggcgttacagttcgaaatttttgatgatgccaattttctggtgctggtcgaaccgcgcattattctgcaggcgcccgatgactatctgttagctggcattggcgatacgctggcgaaatggtatgaagccgttgtgcttgcgccgcagcctgaaacgttgcctttgaccgtaagactgggcattaacagcgcgtgcgctattcgcgatctcctgctgaccagcagcgaacaagcattagcggataaacagcagcgtcggctgacccaggcattttgcgacgtggtggatgcgattatcgctggcggcggtatggtcggcggcctcggggaacgctatacccgtgtcgccgccgcccatgcggtacacaacggtctgaccgtcctgccgcaaacggaaaaattcctgcacggaacgaaagtggcttatggcattctggtgcaaagcgcgctactgggacaagatgacgtgctggcgcaattgattgcagcgtaccggcgatttcatctgccggcccgacttagcgaactggacgtggatattcataacaccgccgagatcgatagggtaatcgcacataccctgcgcccggtcgaatccatccactatttaccggtgacgttaacgcctgacaccctgcgtgcggcgtttgaaaaagttgaatttttcagaatatag

SEQ ID NO: 108 - protein, 362 aa, Salmonella enterica
MNHTEIRVVTGPANYFSHAGSLGRLTDFFTPEQLSHAVWVFGERAIAAARPYLPEAFERAGAKHLQFTGHCSERHVAQLAHACNDDRQVVIGVGGGALLDTAKALARRLALPFVAIPTIAATCAAWTPLSVWYNDAGQALQFEIFDDANFLVLVEPRIILQAPDDYLLAGIGDTLAKWYEAVVLAPQPETLPLTVRLGINSACAIRDLLLTSSEQALADKQQRRLTQAFCDVVDAIIAGGGMVGGLGERYTRVAAAHAVHNGLTVLPQTEKFLHGTKVAYGILVQSALLGQDDVLAQLIAAYRRFHLPARLSELDVDIHNTAEIDRVIAHTLRPVESIHYLPVTLTPDTLRAAFEKVEFFRI

SEQ ID NO: 109 - DNA, 1101 bp, Burkholderia xenovorans
atgaccgccatgatgaaagccgctgtattcatcgagccgggccgcatcgaactcgccgacaaacccgtgcccgatgtcggcgccaacgacgcgctcgtgcgcatcaccaccaccacgatctgcggcaccgacgtgcatatcctgaaaggcgaatacccggttgccaagggcttgactgtcggccacgagcccgtgggcgtgatcgaaaaactcggcagtgcggtgctcggctaccaggagggccagcgagtggtcgcgggcgctatctgccctaacttcaactcctatgccgcgcaagacggggtcccgtcgcaagacggcagttatctgattgccagaggcctgtgcggttgccacggctataaggctacggcgggctggcgctttggcaacatgatcgacggcacgcaggccgaatacgtacttgtccccgatgcgcaggccaatctggcacccgtgcccgacggattgaccgacgaacaggtgttgatgtgtcccgacatcatgtccacgggcttcaagggcgcggaaaacgccaacatccgtatcggcgacacggtcgccgtgttcgcacaagggcccatcggcctgtgtgccacggcgggcgcgcgtctgctcggcgcgagcaccatcatcgcggtggatggaaacgaccaccgtctcgagatagcccggaagatgggggccgacgtcacgctcaacttcaagaattgcgatgtggtggacgaaatcctgaagctcactggcgggcgcggcgtggattcgtcgattgaagcgctcggcacccagagtacgttcgagtcggctttgcggatcctcaaaccaggcggcacgctgtcgagcctcggtgtgtactcgtccgatctgacgattcccctcggggccttcgccgcgggtctcggcgatcataagatcaacaccgcgctgtgtcccggtggcaaggaacgtatgaggcgtttgctgaacgtcgtggaatccggacgcgtcgaccttggcgcactggtcacgcaccactacaagctggacgatatcgtcgctgcatacgacctgtttgcgaatcagcgcgacggggtgcttaaggtggcgatcaagccgcattga

SEQ ID NO: 110 - protein, 366 aa, Burkholderia xenovorans
MTAMMKAAVFIEPGRIELADKPVPDVGANDALVRITTTTICGTDVHILKGEYPVAKGLTVGHEPVGVIEKLGSAVLGYQEGQRVVAGAICPNFNSYAAQDGVPSQDGSYLIARGLCGCHGYKATAGWRFGNMIDGTQAEYVLVPDAQANLAPVPDGLTDEQVLMCPDIMSTGFKGAENANIRIGDTVAVFAQGPIGLCATAGARLLGASTIIAVDGNDHRLEIARKMGADVTLNFKNCDVVDEILKLTGGRGVDSSIEALGTQSTFESALRILKPGGTLSSLGVYSSDLTIPLGAFAAGLGDHKINTALCPGGKERMRRLLNVVESGRVDLGALVTHHYKLDDIVAAYDLFANQRDGVLKVAIKPH

SEQ ID NO: 111 - DNA, 1059 bp, Thermoanaerobacter brockii
atgaaaggttttgcaatgctcagtatcggtaaagttggctggattgagaaggaaaagcctgctcctggcccatttgatgctattgtaagacctctagctgtggccccttgcacttcggacattcataccgtttttgaaggcgccattggcgaaagacataacatgatactcggtcacgaagctgtaggtgaagtagttgaagtaggtagtgaggtaaaagattttaaacctggtgatcgcgttgttgtgccagctattacccctgattggcggacctctgaagtacaaagaggatatcaccagcactccggtggaatgctggcaggctggaaattttcgaatgtaaaagatggtgtttttggtgaattttttcatgtgaatgatgctgatatgaatttagcacatctgcctaaagaaattccattggaagctgcagttatgattcccgatatgatgaccactggttttcacggagctgaactggcagatatagaattaggtgcgacggtagcagttttgggtattggcccagtaggtcttatggcagtcgctggtgccaaattgcgtggagccggaagaattattgccgtaggcagtagaccagtttgtgtagatgctgcaaaatactatggagctactgatattgtaaactataaagatggtcctatcgaaagtcagattatgaatctaactgaaggcaaaggtgtcgatgctgccatcatcgctggaggaaatgctgacattatggctacagcagttaagattgttaaacctggtggcaccatcgctaatgtaaattattttggcgaaggagaggttttgcctgttcctcgtcttgaatggggttgcggcatggctcataaaactataaaaggcgggctatgccccggtggacgtctaagaatggaaagactgattgaccttgttttttataagcgtgtcgatccttctaagctcgtcactcacgttttccggggatttgacaatattgaaaaagcctttatgttgatgaaagacaaaccaaaagacctaatcaaacctgttgtaatattagcataa

SEQ ID NO: 112 - protein, 352 aa, Thermoanaerobacter brockii
MKGFAMLSIGKVGWIEKEKPAPGPFDAIVRPLAVAPCTSDIHTVFEGAIGERHNMILGHEAVGEVVEVGSEVKDFKPGDRVVVPAITPDWRTSEVQRGYHQHSGGMLAGWKFSNVKDGVFGEFFHVNDADMNLAHLPKEIPLEAAVMIPDMMTTGFHGAELADIELGATVAVLGIGPVGLMAVAGAKLRGAGRIIAVGSRPVCVDAAKYYGATDIVNYKDGPIESQIMNLTEGKGVDAAIIAGGNADIMATAVKIVKPGGTIANVNYFGEGEVLPVPRLEWGCGMAHKTIKGGLCPGGRLRMERLIDLVFYKRVDPSKLVTHVFRGFDNIEKAFMLMKDKPKDLIKPVVILA

SEQ ID NO: 113 - DNA, 1056 bp, Clostridium beijerinckii
atgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagacaagccaaaagacttaattaaagcagtagttatattataa

SEQ ID NO: 114 - protein, 351 aa, Clostridium beijerinckii
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

SEQ ID NO: 115 - DNA, 2637 bp, Lactobacillus reuteri
atgcctgctaacaacaaaaaacaagttgaaaaaaacgtattaactgaagaagaaaaaaagcaaaatgcgcaaaaattagttaatgacataattgccaaaagtgaagcagcttttgaacaattacgttactactcacaagaacaagttgataaaatttgtcaagccatggccttagctgctgaagaacaccacatggacttagctattgatgcagctgaagaaactggtcgtggagttgccgaagataaggctatcaagaacatctacgcaagtgaatacatttggaacaacatccgtcatgataagactgtcggaatcatcgaagacaacgatgaaaaccaaaccatcactattgctgatccgcttggtattattgcaggtatcgttccagttactaaccctacttcaacaacgatcttcaaatcaatcattagtgctaagacgcggaatacaattatcttctcattccaccgtcaagccatgaaatcttccattaaaacagctaagattttacaagaagctgcggaaaaggccggtgcaccaaagaacatgattcagtggcttcctgaaagtagccgtgaaaatacaagtgcattgttacaacaccctaagactgctactattttagcaactggtggtccttcattagttaaggctgcctacagttcaggtaaccctgctcttggtgttggtcctggtaacggtccagcttacattgaaaagactgctaatattgaacgttctgtttacgatatcgttctttctaagacttttgataacggaatgatctgtgctactgaaaactcagttgttgttgatgaagaaatctacgacaaggtaaaagaagaattccaaaaatggaactgttacttcttgaagccaaatgaaattgataagttcactgaaggcttcattgatcctaagcgtcaccaagttcgtggaccaattgctggtcgttcagctaacgccatcgctgatatgtgtggaattaaagttcctgaaaacactaaggttatcatcgctgaatatgaaggggttggtgacaagtacccactttcagctgaaaagctttcaccagtattaacaatgtataaggccacttcacatgaaaatgcctttgatatctgtgctcaattattacactacggtggtgaaggtcacactgctgctattcacacccttgatgatgatttagcaactaagtacggtcttgaaatgcgtgcttcacggatcattgttaactctccatctggtatcggtggtattggtaacatttacaataacatgacaccatcacttactttaggtaccggttcatacggtggtaactcaatttctcacaacgttactgattgggatctcttaaacatcaaaacaattgcaaagcggcgtgaaaaccgtcaatgggttaagattcccccaaaagtatactttcaacgcaactcactaaaagaattgcaagatattccaaacattaaccgagcatttatcgttactggtcctggaatgagcaagcgtggttacgttcaacgtgttatcgatcaattgcgtcaacgtcaaaacaacactgctttcttagtatttgatgacgttgaagaagatccatcaacaaacactgttgaaaaaggtgttgccatgatgaacgacttcaaacctgatacaattattgctcttggtggtggttcaccaatggatgctgctaaggctatgtggatgttctatgagcatccagaaacttcatggtatggggttatgcaaaagtatcttgatattcggaagcgtgcttaccaaatcaagaagcctactaagtctcaacttattgggattcctactacatcaggtactggttcagaggttactccatttgcggttattaccgattcagaaactcatgttaagtacccacttgctgactacgccttaactccaaacattgcgattgttgactcacaattcgttgaaactgtcccagcaaaaactactgcatggactggactagatgttttatgtcacgctactgaatcatatgtttcagttatggcaactgattacactcgtggttggtcactacaaaccatcaagggtgttatggaaaaccttcctaagtcagttcaaggtgacaagttagctcgtcgtaagatgcacgacttctcaacaatggccggtatggcatttggtcaagccttcttaggaattaatcactctcttgcccacaagatgggtggagcattcggtcttcctcacggtttgcttatcgctattgcaatgccacaagtaattcgctttaacgcaaaacgtccacaaaagcttgctctctggcctcactatgagacttaccatgcaactaaggactacgctgacattgcacggttcattggtttgaaaggcaacactgatgaagaattagctgaagcatatgctaagaaagttatcgaacttgctcacgaatgtggtgttaagcttagtcttaaggacaatggcgttacacgtgaagaatttgataaagtagttgacgatcttgctcgcttagcttacgaagatcaatgtactactactaacccagttgaaccacttgttagccaactcaaggaattacttgaacgttgctacgatggtactggcgttgaagaaaaataa

SEQ ID NO: 116 - protein, 878 aa, Lactobacillus reuteri
MPANNKKQVEKNVLTEEEKKQNAQKLVNDIIAKSEAAFEQLRYYSQEQVDKICQAMALAAEEHHMDLAIDAAEETGRGVAEDKAIKNIYASEYIWNNIRHDKTVGIIEDNDENQTITIADPLGIIAGIVPVTNPTSTTIFKSIISAKTRNTIIFSFHRQAMKSSIKTAKILQEAAEKAGAPKNMIQWLPESSRENTSALLQHPKTATILATGGPSLVKAAYSSGNPALGVGPGNGPAYIEKTANIERSVYDIVLSKTFDNGMICATENSVVVDEEIYDKVKEEFQKWNCYFLKPNEIDKFTEGFIDPKRHQVRGPIAGRSANAIADMCGIKVPENTKVIIAEYEGVGDKYPLSAEKLSPVLTMYKATSHENAFDICAQLLHYGGEGHTAAIHTLDDDLATKYGLEMRASRIIVNSPSGIGGIGNIYNNMTPSLTLGTGSYGGNSISHNVTDWDLLNIKTIAKRRENRQWVKIPPKVYFQRNSLKELQDIPNINRAFIVTGPGMSKRGYVQRVIDQLRQRQNNTAFLVFDDVEEDPSTNTVEKGVAMMNDFKPDTIIALGGGSPMDAAKAMWMFYEHPETSWYGVMQKYLDIRKRAYQIKKPTKSQLIGIPTTSGTGSEVTPFAVITDSETHVKYPLADYALTPNIAIVDSQFVETVPAKTTAWTGLDVLCHATESYVSVMATDYTRGWSLQTIKGVMENLPKSVQGDKLARRKMHDFSTMAGMAFGQAFLGINHSLAHKMGGAFGLPHGLLIAIAMPQVIRFNAKRPQKLALWPHYETYHATKDYADIARFIGLKGNTDEELAEAYAKKVIELAHECGVKLSLKDNGVTREEFDKVVDDLARLAYEDQCTTTNPVEPLVSQLKELLERCYDGTGVEEK

SEQ ID NO: 117 - DNA, 2595 bp, Lactobacillus sake
atggttaaaaaagaaggcgttaaagcggttgttgatgcggtatctgaagtggataagatggtgacggatttagtcacacgggcccatgaagcacttaaaatcatggaaacattcgatcaagcaaaggtggatcatattgtccaccaaatggcgattgccggccttgatcatcatatggaattagctaaaatggccgttgaagaaacgggtcgtgggatttatgaagataaagccattaaaaatattttcgcaacggaagaaatttggcacgcgattaaagataataagacagttggtgtcattgaagaagatcctgaacatgggattactaagattgcggaaccagtcggggtcattgctggggtaacacctgttaccaacccaacttcaaccacaatttttaaggcagagattgccattaaaacacggaatccaatcatctttgcttttcatcccaatgctcaaaaatgttctgccagagcactagaagtcattaaagaagaagccgttaaagctggcttaccagctgacgccttattattcattgaagaacctagtttggcagccacccaagctttgatgaatcataccggcattgcaacggtcctagcaactggtggaccagggatggtcaaggctgcttattcaactgggaaaccagcattaggggttggggctggtaacgcaccagcttatatcgaagaatcagccaatatcaaacaagcagtcaatgatttaattctatcgaaatcatttgataacgggatgatctgcgcatcagaacaagcagtgattgtcgatgctaaaatctacaacgaagttaaaaaagaattccaagcacaaggtgtctattttgctaaagcaagtgaattaccagctttaaacgaagcaattattgatccagctaaaaacgctgtgcggccagcaattcctggtcaatcagctgctaatattgctaagttagctgggattgatattccagaagatacacccgttttaattgctgaaatcaagggtgttggtcatcaatatccactttcacatgaaaaattatcaccagtcttagcgatgattaaagctaaagaccgtgaagatgggttggcattatgcgaggcgatgcttgatctgggtggcttgggccacacagcatcattgcatacgactgacgatgcgttaccattagaatttgcaagacggatgaaggcttgtcgtgtattggtcaatacaccatctgcccaaggtgggattggtgatttatacaacgaaatgattccatcattaacacttggctgtggctcttatgggcataactcaatttcacataacgtctcaacaatcgacttattgaatattaaaacattggcgaaacgccgcaataatatgcaatgggtcaaattgccaagcaaaatctattttgaaaaaaattcggtcaactatttagaaaagatggctgatttaaataaggtttttatcgtcgctgatcaggggatggttaaccttggttatgtccggattgttgaagaagtgttagctaaacgggcgaatgacgttcaaatgcaaatcttctcagatgttgaaccagatccatcaacaaacacgatttataagggtgctgcagcgatgcggagttttgaaccagatgcaatcgtcgcaattggtggcggttctgtcatggatgctgctaaagggatgtggttattctatgattcagaagaagccgacttctttggcgcaaaacaaaaattcttagacattcgcaaacggacttacaagttccctaaacttaacaaaacgaagttaatctgtatcccaacgacttccgggacgggttcagaagtaacaccttttgcggtgattactgatagtgaaacccatattaaatacccattggcagattatgcattgacaccagacgttgcgattgttgacagtcaatttgtcgagagtgtgccaccacgagttgttgcacacactgggttggatgtgctctgtcatgcgactgaaagttacgtatcaacaatggcttctaactacacgaaggggcttagtttacaggcgattaaattagtttttgataacttgaaggctagttacgatggcgatattacggccaaaggtaacatgcacgatgcctcaacaatggcggggatggcctttgcgaatgcgttattaggcatcaaccattcactggcacataaattaggcggggcgttcaacttacctcacggtttaatgattgccatcacgatgccgcatgttatccgctataatgcgacaacgccaaccaaacgggcactctgggctaaatatagttacttcagagctgatgaagattacgccgaaatcgcacgttatattgggttgaagggtaatacgacagcagaacttgttgaagcttatgcaaatgcagtgactgaattggccgaaagtgttggcattcaaatgagtttgaaagctaatggcgtcacaaaggcggacttcaagcaacatgtggatgagttagctgaattagcttatgaagacaactgtactgttacaaatccaaaagaaccattgattaaagaattaaaaggcattttagaggcagaattttaa

SEQ ID NO: 118 - protein, 864 aa, Lactobacillus sake
MVKKEGVKAVVDAVSEVDKMVTDLVTRAHEALKIMETFDQAKVDHIVHQMAIAGLDHHMELAKMAVEETGRGIYEDKAIKNIFATEEIWHAIKDNKTVGVIEEDPEHGITKIAEPVGVIAGVTPVTNPTSTTIFKAEIAIKTRNPIIFAFHPNAQKCSARALEVIKEEAVKAGLPADALLFIEEPSLAATQALMNHTGIATVLATGGPGMVKAAYSTGKPALGVGAGNAPAYIEESANIKQAVNDLILSKSFDNGMICASEQAVIVDAKIYNEVKKEFQAQGVYFAKASELPALNEAIIDPAKNAVRPAIPGQSAANIAKLAGIDIPEDTPVLIAEIKGVGHQYPLSHEKLSPVLAMIKAKDREDGLALCEAMLDLGGLGHTASLHTTDDALPLEFARRMKACRVLVNTPSAQGGIGDLYNEMIPSLTLGCGSYGHNSISHNVSTIDLLNIKTLAKRRNNMQWVKLPSKIYFEKNSVNYLEKMADLNKVFIVADQGMVNLGYVRIVEEVLAKRANDVQMQIFSDVEPDPSTNTIYKGAAAMRSFEPDAIVAIGGGSVMDAAKGMWLFYDSEEADFFGAKQKFLDIRKRTYKFPKLNKTKLICIPTTSGTGSEVTPFAVITDSETHIKYPLADYALTPDVAIVDSQFVESVPPRVVAHTGLDVLCHATESYVSTMASNYTKGLSLQAIKLVFDNLKASYDGDITAKGNMHDASTMAGMAFANALLGINHSLAHKLGGAFNLPHGLMIAITMPHVIRYNATTPTKRALWAKYSYFRADEDYAEIARYIGLKGNTTAELVEAYANAVTELAESVGIQMSLKANGVTKADFKQHVDELAELAYEDNCTVTNPKEPLIKELKGILEAEF

SEQ ID NO: 119 - DNA, 2667 bp, Giardia intestinalis
atgtcgttgtcggactttgactacggccaggagctcgttgagaccccagaggagctcaacgccctctttgagaaagtggaggaggcagcccatgcgttccgccagcttgaccaggcgcaggtcgacaagatcttctatgccgccgctttcgcagcctcgaaccagcgtatccccttggcaaagatggcctacgaggagacgaatatgggtgtcgtcgaggacaaggtcattaagaacatgtttgggtcggaatatgtgtataacaagtacaagaacatgaagaccgctgggattatcgaggaagacaaggcgggcaatacaattactgttgcagaccctcttggcatcctcgctggtattgtgcccaccacaaacccgacgtccactgccatcttcaagtgccttatagcgctcaagacgaggaactgcatcatcttctctccgcatcacagggccgtcaagtcaaccatccacggcccacgcattgtccgtgacgccgccgtcaaagctggagcacctcccaattgcattgcctggattacgaagccttccgttccccttgcgaaggctctcatggggcaccccaagactagctgtgtcctcgctactggcggccccggcatggtcacttcggcctatagctccggcaacccctccattggtgtcggtccgggtaatgtccctgcgctcattgacgagacgtgtgactacaagaccgccgtgaaccaggttatcaatagcaagtcgttcgacaatggcgtcgtctgcgcttcagaacaagccatcgtctgtgtcacgaaggagatctacgacaagtgcatcgaggagctcaagttccgcggcgcctacgtcatgacggaaggacgagaagcagcgtgtcaacaagctgatcctgcactgaacgaggccactgggaaacatcagctcaactgcgacatagtcggacgccctgcccgcgacatcgctgcaaatggcagcgtcgtcgttcctgcggactgcaagtgccgctgcatcgtgggaaccttcacagaggtgcgccacgacgaggcgatgtcctgcgaaaagctctccccagttttgggtatctgttgggctgagacattcgagaaggccgtcgacatttgcggacagatgatcgatatggctggggcaggccacacagctgcgattcacacagcgccccacagacgcgaccgcattgagtacttcacgcaccacatcagggctggccgcattgtcgttaactctccgagcacgtttggaggtatcggcgatctctacaactttgcgatcgatcccaccatgactatcggctgtggctcatatggcaagaactccgtctctgagaacgtcgggccgaagcaccttctcaactataagaaggtggccattgtgaggcgcaatccgctctggttcaaggttccgcaggtgatgcacgtcggtgagggcgctcttgctaaggctgctgcggacctcatctcccgcgggctctccagggcttatatcatcactggaaaggtcatgcacgatcttgggttcacggacaagatcatcagcccccttactgcaggcaacgttactgtcaaggtcttcacagacgtcctgccggaccctgaccttgggacctgctataggagccttgcagaggtcagggatttccaaccggacatgattatcgctcttggcggtggctctgcgatggatctggcgaagatggttaggcttctgtatgagcatccgaaggtagactttgccgggcttgcgcagcgcttcatggacatccgcaagcgcatctacgagtatcccgagtgcctggatcttcgcacagcgaaaacgttcagtgtcgcaatcccgacgacttctggaacaggctctgaggtgaccccgttctctgtcatcacggacgagaaggagcacgtcaagtatccccttgcggactaccagctcatgacgcacatggctgtcattgatccagagcttgtcctgaccgttcctgcatcacttgcttcctggacgggcgtcgacgccctaactcacgctattgagtcgtacgtgtccgttatggcgactgagtacaccatgcccctctctctccaagcaatcaagacagtcttcgagaacctcgagaaatctgtcgtcagcaggtgcccgactgctcgcggaaatgtgcatcaggctgcgaccatcgccggcattgcgtttgccaacgctttcctgggtatctgccactcctgcgcccacaagctcggccaaaagtatcacattccgcacgggcttgctaacgcaataatgctcccacacgtcatcaggtacaacgcagtcgacgatcccgtcaagatcgccacgtttccgcagtacctctaccctgttgctctcgagcgctacgctgagatagccgactactgcgggttcacgaacaagaatgacgggaagagtgtcaaggagaagacggagatcctcattaagaagatctacgacctctatgagaaggtagggattgacgcgaagatcagtgcgtgcaaggaggctcctgttgaggcagacttcttcgccgaggagaacctggactatctcgcataccacgcctttgacgaccagtgcaccggtgccaatccgcgctacccgctgatcgaggacttcaaggagctcttccgtgcggcctggtaa

SEQ ID NO: 120 - protein, 888 aa, Giardia intestinalis
MSLSDFDYGQELVETPEELNALFEKVEEAAHAFRQLDQAQVDKIFYAAAFAASNQRIPLAKMAYEETNMGVVEDKVIKNMFGSEYVYNKYKNMKTAGIIEEDKAGNTITVADPLGILAGIVPTTNPTSTAIFKCLIALKTRNCIIFSPHHRAVKSTIHGPRIVRDAAVKAGAPPNCIAWITKPSVPLAKALMGHPKTSCVLATGGPGMVTSAYSSGNPSIGVGPGNVPALIDETCDYKTAVNQVINSKSFDNGVVCASEQAIVCVTKEIYDKCIEELKFRGAYVMTEGREAACQQADPALNEATGKHQLNCDIVGRPARDIAANGSVVVPADCKCRCIVGTFTEVRHDEAMSCEKLSPVLGICWAETFEKAVDICGQMIDMAGAGHTAAIHTAPHRRDRIEYFTHHIRAGRIVVNSPSTFGGIGDLYNFAIDPTMTIGCGSYGKNSVSENVGPKHLLNYKKVAIVRRNPLWFKVPQVMHVGEGALAKAAADLISRGLSRAYIITGKVMHDLGFTDKIISPLTAGNVTVKVFTDVLPDPDLGTCYRSLAEVRDFQPDMIIALGGGSAMDLAKMVRLLYEHPKVDFAGLAQRFMDIRKRIYEYPECLDLRTAKTFSVAIPTTSGTGSEVTPFSVITDEKEHVKYPLADYQLMTHMAVIDPELVLTVPASLASWTGVDALTHAIESYVSVMATEYTMPLSLQAIKTVFENLEKSVVSRCPTARGNVHQAATIAGIAFANAFLGICHSCAHKLGQKYHIPHGLANAIMLPHVIRYNAVDDPVKIATFPQYLYPVALERYAEIADYCGFTNKNDGKSVKEKTEILIKKIYDLYEKVGIDAKISACKEAPVEADFFAEENLDYLAYHAFDDQCTGANPRYPLIEDFKELFRAAW

SEQ ID NO: 121 - DNA, 2604 bp, Shewanella amazonensis
atgacagtcaccaacactcaggaactcaacgaactggtcgcgcgcgtcgccaaggcacaggcccaatttgcttcttacagccaagagcaagtggacaggattttccgcgccgccgccctcgccgctgctgatgcacgcattcgtctggccaaaatggccgctgaagaaacccgcatgggggttgttgaagataaggtcattaaaaaccatttcgcctccgagtacatttacaacaaatacaaagacgaaaagacctgcggcattctggccgaagacgccaccttcggcactatcaccatcgccgagccggttgggatcatctgcggcattgtgcccaccaccaacccgacctccaccgctatctttaaagcgctgataagcttaaagacccgcaacggcattatcttctcgcctcacccaagggccaaggtttcaactaccactgccgcccgcatcgtactggatgcagccattgcagcaggtgccccgaaagacattattggctggattgatgagccttcggtggcgctgtccaaccagttgatgacccacgagaagattaaccttattctcgccaccggcggccccggcatggtgaaagcggcctactcctccggcaaacccgcgattggcgttggcgccggcaacacccccatcgtgattgatgagaccgctgacatcaaacgtgctgtaagctcaattttgatgtccaagaccttcgataacggcgtggtgtgtgcctccgagcaagcggttgtggtggtggatgcggtttacgatgcggtaaaagagcgcttctcgagccacggcggctatctgctcagcaagaaagaaaacgccgcgctgcaaaaagtcatcctcaaagacggtggcctcaatgccgatatcgtgggtcaaagcgccgccaccattgccgccatggccaatatcaaggtgcctgcccacaccaaggtgctgattggtgaagtcaccgacatcgatgagaaagaggccttcgcccacgagaaactctcaccgctgcttggcatgtaccgcgccgccaactttgaagaggcgctggacaaggccgaagccctggttgctcttggcggtattggtcacacctcaggcctgtataccgatcaggatacccaggacgagcgggtcaaatccttcggctaccggatgaagaccgcccgtattctgattaacaccccggcctctcagggcggcataggcgatttatacaacttcaagctggcgccttcattgactctgggctgcggctcctggggcggtaactcgatttctgaaaacgtaggtccaagccatcttatcaacaagaaaaccgtcgccaagagggccgaaaatatgctgtggcacaagcttccttcgtccatctacttccgccgtggcagcctgccgattgccctcgaagagctgagtggcaagaagcgtgcgcttatcgtgaccgacaagtttctgttcaacaacggctattgcgatgaaaccatccgaattctgaagtcacagggcctggaaaccgaggtcttttacgaagtggaagccgacccaaccttagcggtggttcgcgccggagccaaagtggcaaccagcttccaacctgatgtgatagtggctctgggtggtggttcgcccatggatgcggccaagatcatttgggtaatgtacgagcatcccgatgtggactttgccgacctggcgctgcgctttatggatatccgtaagcgtatctacaagttccccaaacttggtgccaaggcaatgatggtggccattcccaccacctcaggtactggctctgaagtcacgccttttgcggtggtgaccgacgagcaaaccggtgccaaataccccattgccgactatgaactgacccccaacatggccattgtcgaccccaatctggtaatggatatgcccaagtcgctcaccgctttcggcggtatcgatgccattacccacgcactggaagcctatgtcagcgtgatggccaacgaatacagcgacggccaggcactgcaggcactggatttactgtttaagtatctgccagacagctacgccaggggcgcgcaggcgccgctggcacgggaaaaagtgcacaatggcgccaccattgccggtatcgcttttgccaacgccttcttaggcatttgccactcaatggctcacaaattgggcgctgagtttcacctgccccatggccttgccaatgcgctccttatcagcaacgtcattcgcttcaatgccactgacctgcccaccaaacaggcggcgttcagccagtacgacagacccaaggccctgtgccgttatgccgccattgccagccacttaggccttgccggcaacaatgatgaagccaaggtagaggcgcttatcgccaagattgaggagcttaaagccgccatcggtattccggtgtctatcaaggacgcaggggtaaatgaagccgactttatggccaagcttgatgagttggccgaagatgcctttgacgatcagtgcaccggcgccaacccgcgctacccactgattagcgagctgaaacagctgcttatcgatagcttccatggacgtgcctatcaggacagtctctga

SEQ ID NO: 122 - protein, 867 aa, Shewanella amazonensis
MTVTNTQELNELVARVAKAQAQFASYSQEQVDRIFRAAALAAADARIRLAKMAAEETRMGVVEDKVIKNHFASEYIYNKYKDEKTCGILAEDATFGTITIAEPVGIICGIVPTTNPTSTAIFKALISLKTRNGIIFSPHPRAKVSTTTAARIVLDAAIAAGAPKDIIGWIDEPSVALSNQLMTHEKINLILATGGPGMVKAAYSSGKPAIGVGAGNTPIVIDETADIKRAVSSILMSKTFDNGVVCASEQAVVVVDAVYDAVKERFSSHGGYLLSKKENAALQKVILKDGGLNADIVGQSAATIAAMANIKVPAHTKVLIGEVTDIDEKEAFAHEKLSPLLGMYRAANFEEALDKAEALVALGGIGHTSGLYTDQDTQDERVKSFGYRMKTARILINTPASQGGIGDLYNFKLAPSLTLGCGSWGGNSISENVGPSHLINKKTVAKRAENMLWHKLPSSIYFRRGSLPIALEELSGKKRALIVTDKFLFNNGYCDETIRILKSQGLETEVFYEVEADPTLAVVRAGAKVATSFQPDVIVALGGGSPMDAAKIIWVMYEHPDVDFADLALRFMDIRKRIYKFPKLGAKAMMVAIPTTSGTGSEVTPFAVVTDEQTGAKYPIADYELTPNMAIVDPNLVMDMPKSLTAFGGIDAITHALEAYVSVMANEYSDGQALQALDLLFKYLPDSYARGAQAPLAREKVHNGATIAGIAFANAFLGICHSMAHKLGAEFHLPHGLANALLISNVIRFNATDLPTKQAAFSQYDRPKALCRYAAIASHLGLAGNNDEAKVEALIAKIEELKAAIGIPVSIKDAGVNEADFMAKLDELAEDAFDDQCTGANPRYPLISELKQLLIDSFHGRAYQDSL

SEQ ID NO: 123 - DNA, 2658 bp, Thermosynechococcus elongatus
atgaatgccccaaccttgaccagtgacccccccgttcaaagccttgccgatctggaagggctgattgagcgcgtccaacgggcgcagagtcagtacgcccaatttacccaagagcaagtggatcacattttccacgaagcagccatggcggccaaccaagcccggattcccctggccaaacaagccgtagccgaaacgggcatgggggttgtcgaagataaagttattaaaaatcactttgcttcggaatacatctacaacaagtacaaaaatgagaaaacctgcggcgtcattgaggatgaccccatctttggtatccaaaaaattgctgaaccggtggggatcattgccggtgtggtgccggtcacgaaccccacttcaacgaccatctttaaggcactgattgccctgaagactcgcaatggcattatcttttcgccccacccccgggcaaaggcctgtacggttgcagcggccaaggtagtgttggatgcagcggtcgctgccggcgcaccccccgatattattggctggattgatgagccgacgattgaactctcccaagccctgatgcagcacccgcagatcaagctgattttggccacggggggaccaggtatggtcaaggcagcctattcctctggccatccggcgatcggggtcggggccgggaatacccccgtgctcattgatgccacagccgatattcccacggcagtgagttcgattctcctcagtaaggcctttgacaatggcatgatctgtgcctcggagcaggcagtgattgttgtggatgagatttatgacgcacttaaagctgagtttcaacggcgaggggcctaccttctctcccctgaggaacggcagcaggtggcacaactactgctgaaggatggtcgcctcaatgccgccattgttggtcaatcggccgccaccattgccgcaatggccaatatccaagtaccgccagaaacccgggtactcattggcgaggtgagtgaagtggggccgcaggagccattttcctatgagaaactctgtccggtattggcgttatatcgggcaccccagttccataaaggggtggagattgcggcccagttggtgaattttgggggcaaggggcatacatctgtgctctataccgatccccgcaatcaagatgatattgcctatttcaaataccgcatgcaaacggcgcgggttctgattaacaccccttcttcccagggggcaattggcgatctctacaacttcaagttagatccgtcgctaacccttggttgtggtacgtggggcggcaacgtcacatcggaaaatgttggtccccgtcacttgctgaatattaaaacggtgagcgatcgccgggaaaatatgctttggtttcgggtgccgcccaagatctacttcaaacccggctgtttgcccattgccctgcgggagctggcggggaaaaaacgcgccttcctcgtgacggataaacccctctttgacttggggatcactgaaccgattgtccataccctcgaagaactgggcatcaagtatgacatcttccatgaagtggaaccagatccaaccctcagtaccgttaaccgcggtctagggttgctgcggcaatatcagccggatgtgattgttgctgtggggggtggctcacctatggatgcagccaaggtgatgtggctgttgtatgagcatccggaggtggagtttgacggccttgcgatgcgcttcatggatattcgcaagcgggtgtatcaactgcctcccttgggtcaaaaggcaatcctggtggctattcccaccacctcggggacgggttcagaggtgaccccctttgccgtggttaccgacgatcgcgtggggattaaatatcccttggcagactatgcccttacgccaacgatggcgattgtggatcccgacttggtgctgcacatgcccaagaaactgacggcctacggtggcattgatgcgctgacccatgccctggaggcctatgtgtcggtgctctcgacggagtttacggagggactggctctagaggccattaaactgctctttacctacctaccccgtgcctatcgcttgggggcggcggatccggaggcacgggagaaggtccactatgcggcgacgatcgctggcatggcctttgcgaatgccttcttgggggtctgccactcgctggcccacaaactaggctccaccttccacgtgccccacggcttggcgaatgcactcatgatttcccatgtgattcgctacaatgccacggatgctcccctgaagcaggcgattttcccgcagtacaagtatccccaagcgaaggagcgctatgcccaaattgccgacttcctcgaattggggggcacgaccccagaggaaaaagtggagcgtctcattgcggcaattgaggatttgaaagcccaattagaaattcccgccacgattaaggaggccctcaacagtgaggatcaagcgttctatgagcaggtggagagcatggccgaactggcctttgacgatcagtgcacgggggccaatccccgctatccgctgatccaagacctcaaggagttgtatatcctggcctatatggggtgtcggcgggatgcggcagcctactatgggggggaggcaacggggagttga

SEQ ID NO: 124 - protein, 885 aa, Thermosynechococcus elongatus
MNAPTLTSDPPVQSLADLEGLIERVQRAQSQYAQFTQEQVDHIFHEAAMAANQARIPLAKQAVAETGMGVVEDKVIKNHFASEYIYNKYKNEKTCGVIEDDPIFGIQKIAEPVGIIAGVVPVTNPTSTTIFKALIALKTRNGIIFSPHPRAKACTVAAAKVVLDAAVAAGAPPDIIGWIDEPTIELSQALMQHPQIKLILATGGPGMVKAAYSSGHPAIGVGAGNTPVLIDATADIPTAVSSILLSKAFDNGMICASEQAVIVVDEIYDALKAEFQRRGAYLLSPEERQQVAQLLLKDGRLNAAIVGQSAATIAAMANIQVPPETRVLIGEVSEVGPQEPFSYEKLCPVLALYRAPQFHKGVEIAAQLVNFGGKGHTSVLYTDPRNQDDIAYFKYRMQTARVLINTPSSQGAIGDLYNFKLDPSLTLGCGTWGGNVTSENVGPRHLLNIKTVSDRRENMLWFRVPPKIYFKPGCLPIALRELAGKKRAFLVTDKPLFDLGITEPIVHTLEELGIKYDIFHEVEPDPTLSTVNRGLGLLRQYQPDVIVAVGGGSPMDAAKVMWLLYEHPEVEFDGLAMRFMDIRKRVYQLPPLGQKAILVAIPTTSGTGSEVTPFAVVTDDRVGIKYPLADYALTPTMAIVDPDLVLHMPKKLTAYGGIDALTHALEAYVSVLSTEFTEGLALEAIKLLFTYLPRAYRLGAADPEAREKVHYAATIAGMAFANAFLGVCHSLAHKLGSTFHVPHGLANALMISHVIRYNATDAPLKQAIFPQYKYPQAKERYAQIADFLELGGTTPEEKVERLIAAIEDLKAQLEIPATIKEALNSEDQAFYEQVESMAELAFDDQCTGANPRYPLIQDLKELYILAYMGCRRDAAAYYGGEATGS

SEQ ID NO: 125 - DNA, 2577 bp, Clostridium acetobutylicum
atgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaa

SEQ ID NO: 126 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 127 - DNA, 2580 bp, Clostridium carboxidivorans
atgaaggtaactaatgttgaagaactgatgaaaaaaatgcaggaagtgcaaaatgctcaaaaaaaatttgggagttttactcaggaacaagtagatgaaattttcaggcaagcagcactagcagctaacagtgccagaatagatctagctaaaatggcagtggaagaaactaaaatgggaattgtagaggataaggttataaaaaatcattttgttgcagaatacatatataataagtataaaaatgaaaaaacttgtgggattttggaagaagatgaaggctttggaatggttaaaattgcagaacctgtaggtgtgattgcagcagtaattccaacaacaaatccaacatctacagcaatatttaaagcattattagctttgaaaacaagaaatggtataattttttcaccacatccaagagcaaaaaagtgtactattgcagcagctaagttagttcttgatgctgcagttaaagcaggtgctcctaaaggaattataggttggatagatgaaccttctattgaactttcacagatagtaatgaaagaagctgatataatccttgcaacaggtggtccaggtatggttaaagcagcttattcttcaggtaaacctgctataggggttggtcctggtaacacacctgctttaattgatgaaagtgctgatattaaaatggcagtaaattcaatacttctttccaaaacttttgataatggtatgatttgtgcttcagagcagtcggtagtagttgtagattcaatatatgaagaagttaagaaagaatttgctcatagaggagcttatattttaagtaaggatgaaacaactaaagttggaaaaatactcttagttaatggtacattaaatgctggtatcgttggtcagagtgcttataaaatagcagaaatggcaggagttaaagttccagaagatgctaaagttcttataggagaagtaaaatcagtggagcattcagaagagccattttcacatgaaaagttatctccagttttagctatgtatagagctaaaaattttgatgaagctcttttaaaagctggaagattagttgaactcggtggaatgggtcatacatctgtattatatgtaaatgcaataactgaaaaagtaaaagtagaaaaatttagagaaactatgaagactggtagaacattaataaatatgccttcagcacaaggtgctataggagacatatataactttaaactagctccttcattaacattaggttgtggttcatggggaggaaactccgtatcagaaaatgttggacctaaacacttattaaatataaaaagtgttgctgagaggagagaaaatatgctttggtttagagttcctgaaaaggtttattttaaatatggtagtcttggagttgcattaaaagaattagatattttggataagaaaaaagtatttatagtaacagataaagttctttatcaattaggttatatagatagagttacaaagattcttgaagaattgaaaatttcatataaaatatttacagatgtagaaccagatccaaccctagctacagctaaaaaaggtgcagaagaattgttatcatttaatccagatactattatagcagttggtggtggttcagcaatggatgctgctaagattatgtgggtaatgtatgaacatccggaagtaagatttgaagatttagctatgagatttatggatataagaaagagagtatatacttttcctaagatgggtgaaaaagcaatgatgatttctgttgcaacatcagcaggaacaggatcagaagtaacaccttttgcagtaattactgatgaaaaaacaggagctaaatatccattagctgattatgaattaactccaaatatggctataattgatgctgaacttatgatgggtatgccaaaaggattaacagcagcttcaggaatagatgcactaactcatgcaatagaagcttatgtatcaataatggcttcagaatatactaatggattagcgttagaagcaataagattgatatttaagtatttaccaatagcttacagtgaaggaacaacaagtataaaggcaagagaaaaaatggcgcatgcttcaacaatagctggtatggcatttgctaatgcatttttaggagtatgtcattcaatggcacataaattaggatcaactcatcacgtaccacatggcattgccaatgcactacttataaatgaagttataaaatttaatgcagtagaaaatccaagaaaacaagctgcatttccacaatataagtatccaaatataaaaaagagatatgctagaatagcagattaccttaacttaggtgggtcaacagacgatgaaaaagtacaattattaataaatgctatagatgaattaaaagctaagataaatattccagaaagtattaaagaagcaggagtaacagaagaaaaattttatgctactttagataaaatgtcagaattagcttttgatgatcaatgtacaggtgcaaaccctagatatccattaataagtgaaataaaacaaatgtatgtaaatgcattttaa

SEQ ID NO: 128 - protein, 859 aa, Clostridium carboxidivorans
MKVTNVEELMKKMQEVQNAQKKFGSFTQEQVDEIFRQAALAANSARIDLAKMAVEETKMGIVEDKVIKNHFVAEYIYNKYKNEKTCGILEEDEGFGMVKIAEPVGVIAAVIPTTNPTSTAIFKALLALKTRNGIIFSPHPRAKKCTIAAAKLVLDAAVKAGAPKGIIGWIDEPSIELSQIVMKEADIILATGGPGMVKAAYSSGKPAIGVGPGNTPALIDESADIKMAVNSILLSKTFDNGMICASEQSVVVVDSIYEEVKKEFAHRGAYILSKDETTKVGKILLVNGTLNAGIVGQSAYKIAEMAGVKVPEDAKVLIGEVKSVEHSEEPFSHEKLSPVLAMYRAKNFDEALLKAGRLVELGGMGHTSVLYVNAITEKVKVEKFRETMKTGRTLINMPSAQGAIGDIYNFKLAPSLTLGCGSWGGNSVSENVGPKHLLNIKSVAERRENMLWFRVPEKVYFKYGSLGVALKELDILDKKKVFIVTDKVLYQLGYIDRVTKILEELKISYKIFTDVEPDPTLATAKKGAEELLSFNPDTIIAVGGGSAMDAAKIMWVMYEHPEVRFEDLAMRFMDIRKRVYTFPKMGEKAMMISVATSAGTGSEVTPFAVITDEKTGAKYPLADYELTPNMAIIDAELMMGMPKGLTAASGIDALTHAIEAYVSIMASEYTNGLALEAIRLIFKYLPIAYSEGTTSIKAREKMAHASTIAGMAFANAFLGVCHSMAHKLGSTHHVPHGIANALLINEVIKFNAVENPRKQAAFPQYKYPNIKKRYARIADYLNLGGSTDDEKVQLLINAIDELKAKINIPESIKEAGVTEEKFYATLDKMSELAFDDQCTGANPRYPLISEIKQMYVNAF

SEQ ID NO: 129 - DNA, 252 bp, Propionibacterium acidi-propionici
ccgccccgatcgtcgtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggacccatgagtccgcgaaagattggcgttaccgagctcgcgctccgcgacgcgcatcagagcctgattgcaacccgg

SEQ ID NO: 130 - DNA, 180 bp, Propionibacterium acidi-propionici
ccgccccgatcgtcgtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggaccc

SEQ ID NO: 131 - DNA, 121 bp, Propionibacterium acidi-propionici
tcaggctgagaacgacctgatccgccactcgcggaactccggacgccgcgtcccctcgggggcgcggcgtcctgcatgtccgggcgcaggggcaaggcaggcctcctactcgctgtactcg

SEQ ID NO: 132 - protein, 246 aa, Clostridium beijerinckii
  ref <308>: DATABASE ACCESSION NUMBER: GenBank Q9RPK1
  ref <309>: DATABASE ENTRY DATE: 2012-04-18
  ref <313>: RELEVANT RESIDUES IN SEQ ID NO: (1)..(246)
MLESEVSKQITTPLAAPAFPRGPYRFHNREYLNIIYRTDLDALRKIVPEPLELDGAYVRFEMMAMPDTTGLGSYTECGQAIPVKYNEVKGDYLHMMYLDNEPAIAVGRESSAYPKKFGYPKLFVDSDALVGALKYGALPVVTATMGYKHEPLDLKEAYTQIARPNFMLKIIQGYDGKPRICELICAENTDITIHGAWTGSARLQLFSHALAPLADLPVLEIVSASHILTDLTLGTPKVVHDYLSVK

SEQ ID NO: 133 - protein, 246 aa, Clostridium beijerinckii
  ref <308>: DATABASE ACCESSION NUMBER: GenBank A6M020.1
  ref <309>: DATABASE ENTRY DATE: 2012-05-16
  ref <313>: RELEVANT RESIDUES IN SEQ ID NO: (1)..(246)
MLESEVSKQITTPLAAPAFPRGPYRFHNREYLNIIYRTDLDALRKIVPEPLELDRAYVRFEMMAMPDTTGLGSYTECGQAIPVKYNGVKGDYLHMMYLDNEPAIAVGRESSAYPKKLGYPKLFVDSDTLVGTLKYGTLPVATATMGYKHEPLDLKEAYAQIARPNFMLKIIQGYDGKPRICELICAENTDITIHGAWTGSARLQLFSHALAPLADLPVLEIVSASHILTDLTLGTPKVVHDYLSVK

SEQ ID NO: 134 - protein, 246 aa, Clostridium beijerinckii; identical to SEQ ID NO: 132
  ref <308>: DATABASE ACCESSION NUMBER: GenBank AAD54946.1
  ref <309>: DATABASE ENTRY DATE: 1999-09-14
  ref <313>: RELEVANT RESIDUES IN SEQ ID NO: (1)..(246)

SEQ ID NO: 135 - protein, 246 aa, Clostridium beijerinckii; identical to SEQ ID NO: 132
  ref <308>: DATABASE ACCESSION NUMBER: GenBank AAD54949.2
  ref <309>: DATABASE ENTRY DATE: 2004-05-17
  ref <313>: RELEVANT RESIDUES IN SEQ ID NO: (1)..(246)

SEQ ID NO: 136 - protein, 246 aa, Clostridium beijerinckii
  ref <308>: DATABASE ACCESSION NUMBER: GenBank AAT48942.1
  ref <309>: DATABASE ENTRY DATE: 2004-06-27
  ref <313>: RELEVANT RESIDUES IN SEQ ID NO: (1)..(246)
MLESEVSKQITTPLAAPAFPRGPYRFHNREYLNIIYRTDLDALRKIVPEPLELDRAYVRFEMMAMPDTTGLGSYTECGQAIPVKYNGVKGDYLHMMYLDNEPAIAVGRESSAYPKKLGYPKLFVDSDTLVVTLKYGTLPVATATMGYKHEPLDLKEAYAQIARPNFMLKIIQGYDGKPRICELICAENTDITIHGAWTGSARLQLFSHALAPLADLPVLEIASASHILTDLTLGTPKVVHDYLSVK

SEQ ID NO: 137 - protein, 246 aa, Clostridium beijerinckii; identical to SEQ ID NO: 133
  ref <308>: DATABASE ACCESSION NUMBER: GenBank ABR35950.1
  ref <309>: DATABASE ENTRY DATE: 2012-01-19
  ref <313>: RELEVANT RESIDUES IN SEQ ID NO: (1)..(246)

SEQ ID NO: 138 - DNA, 749 bp, Propionibacterium acidi-propionici
tggatccatagggtggtgcacggggacgaaattttcaatgattctgctgtcgtcaatgaccaggtgctcgcgcaaattgaagatttggcggaactcgcgccgcttcataaccgggcgaacgcaacggggatccgggcgttccgtgccgtgctgccggatgtggttcaagtggccgtgtttgataccgctttccaccaaacgatgccggaaagtgctttcttatacagcctcccctatgcatactacgaaaaataccggatccgcaaatacgggtttcatggcacttcccataaatatgtggcgatgcgtgccgctgagctgctcggcaggccaattgaacagctgcgcctgatttcatgccatttgggcaacggggcaagcattgcggcgatccagggcggccggtcaatcgatacgtccatgggctttacgccattggccggcgtgacgatgggcacgcgctccggcaatatcgaccccgcactgatcccgtttattatggagaagacaggcaaaacggcagaagaagtgctcgaagtgttaaacaaagaatccgggcttctcggcatttcgggcgtttccagcgatttgcgcgatatccaggtggcggcggaactcgagcggaacaagcgggctgaactggcgcttgacatttttgcaagccgcatccataaatacatcggttcgtatgcggcaaaaatggccagcgtcgatgcgattattttcaccgccggcattggcgaggatcc

SEQ ID NO: 139 - protein, 250 aa, Propionibacterium acidi-propionici
WIHRVVHGDEIFNDSAVVNDQVLAQIEDLAELAPLHNRANATGIRAFRAVLPDVVQVAVFDTAFHQTMPESAFLYSLPYAYYEKYRIRKYGFHGTSHKYVAMRAAELLGRPIEQLRLISCHLGNGASIAAIQGGRSIDTSMGFTPLAGVTMGTRSGNIDPALIPFIMEKTGKTAEEVLEVLNKESGLLGISGVSSDLRDIQVAAELERNKRAELALDIFASRIHKYIGSYAAKMASVDAIIFTAGIGEDP

SEQ ID NO: 140 - protein, 218 aa, Clostridium acetobutylicum
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 141 - protein, 221 aa, Clostridium acetobutylicum
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 142 - protein, 392 aa, Clostridium beijerinckii
  ref <308>: DATABASE ACCESSION NUMBER: GenBank ABR35750.1
  ref <309>: DATABASE ENTRY DATE: 2012-01-19
  ref <313>: RELEVANT RESIDUES IN SEQ ID NO: (1)..(392)
MKDVVIVSAVRTAIGAYGKTLKDVPAVELGAIVIKEAVKRANIKPEEINEVIFGNVLQAGLGQNPARQAAVKAGLPIEIPAFTINKVCGSGLRAISLAAQIIKAGDADAIVVGGMENMSSAPFLLDNARWGQRMGHGEFIDEMIKDGLWDAFNDYHMGVTAENVAEKWNVTREEQDEFSLLSQQKAEKAIKSGEFKDEIVPVVIKTKKGEIIFDQDEFPRFGNTIEALKKLKPIFKENGTVTAGNASGLNDGAAALVIMSADKAKALGIKPLAKITSYGSAGLDPAIMGYGAFYATKAALDKINLKAEDLDLIEANEAYASQSIAIARDLNLDMNKVNVNGGAIALGHPIGASGARILVTLLHAMEKRDAKKGLATLCIGGGQGTALIVERE

SEQ ID NO: 143 - protein, 393 aa, Clostridium beijerinckii
  ref <308>: DATABASE ACCESSION NUMBER: GenBank ABR32599.1
  ref <309>: DATABASE ENTRY DATE: 2012-01-19
  ref <313>: RELEVANT RESIDUES IN SEQ ID NO: (1)..(393)
MREVVIVSAVRTALGSFGGALKDVSAVDLGALVIKEAVNRAGVKPELIEEVIMGNVIQAGLGQNTARQSTIKAGLPQEVSAMTINKVCGSGLRAVSLAAQMIKAGDADVVVAGGMENMSAAPYALDKARWGQRMGDGKLVDTMIKDALWDAFNNYHMGVTAENIAKQWGLTREEQDAFSAASQQKAEAAIKSGRFKDEIVPVVIPQRKGEPKVFDTDEFPRFGTTAETLAKLKPAFIKDGTVTAGNASGINDGAAAFVVMSAEKAEELGLKPMAKILSYGSKGLDPAIMGYGPFHATKKALEKANLTVEDLDLIEANEAFAAQSLAVAKDLKFDMSKVNVNGGAIALGHPVGASGARILVTLLHEMEKRDAKKGLATLCIGGGMGTALIVERI

SEQ ID NO: 144 - DNA, 1553 bp, Streptomyces laurentii
  ref <308>: DATABASE ACCESSION NUMBER: GenBank L39157.1
  ref <309>: DATABASE ENTRY DATE: 1996-05-16
  ref <313>: RELEVANT RESIDUES IN SEQ ID NO: (1)..(1553)
ctgatcagccggtcgccatggaggagggcctcaagttcgccatccgtgagggtggccggaccgtcggcgccggccaggtcgtcaagatcgtcaagtaagtccgcttcgctgacggctgattgaccgggtcgctccgaaggagcgctcgaatcacgcgcagcccgtagcgactccggtcgtacgggccctttgtcgtaccggcggcggccggcggggcggatttcccgggcgcgcgggaatgggaaatggcgggtttccccgggaaaacaattcgggggcgaggtccggtcccggcctccggaaagcgtccgaatcggtcacattgcctgtgctaccgtctgcgtcgtggccaatcttgacgttattgtcgaccgttccgaccccgcggtgcagcgcatcgtcgatgtgaccaagcattcccggtccgtcgtgcgaacggtgctgatcgaggacatcgagcccctgacgcagagcatccgcgccggggtcgagttcaccgaggtctacggtctcgacaccgtgccgttcccgggtgatctgctcgccgcctgcgaaaagcgcggaattcgggtgcggctgctctccgccgcggtcgcgaatcaggttttcaagaccgagaagaagcccaaggtcttcggtatcgcgaaggttccgccggccggccgtttcgccgacctggagagcctttccggcgatgtcgtcctgctcgacggcgtgaagatcgtcggcaacatcggcgccatcgtgcggacgcgttcggcgctcggcgccgccggcatcgtcctggtcgacagcggcctcggcaccatcgcggaccgccggctcatccgcgccagccgcggctacgtgttctccctgccgatcgtgctcgcgacgcgcgacgaggcgctggccttcttccgtgacggcgggatgcggcccgtggtcttcgaggcggacggcaagctgtccatcggagagctcgacggcatcgacgagcggctcgtgctcgtgttcggcagcgagaagaccggcccgtcgggcgagttcgccggggtcgccaccgagtcggtgtccatcccgatgaaccccgccgccgagtcgctcaacgtctcggtgtcggccggcatcgccctgcaccggcgggcccgccgcaacctctcgcgcccgcgcggctgagcggccgcctcgcgcgttccggccgcgtccgtacggctcccggcccgcctcccttcgtgggacggcgggcccgcgtcgtctcgcgtcccgttcgtccgctatctgacgtcccgtaacatgcgcgcacatcaggcgtgggccggtcgagcggtggaggcatggtgacagcggtgggtcgtacgcgagggacgcgagggacgcgaggcaggccggcgcgagtgcggacgcgtcgggggacggacgcccgggtccggcgtgcggcggtggccgccgcctcgcggccgccctggtcctgaccccgcaggccgccgcgtacggagtggccggcgaggccgccgccgctcccgggaccgctcccggggccgaacccgggcacgacctcaccggcgggatcc

SEQ ID NO: 145 - DNA, 49 bp, Artificial Sequence
  Oligomer
ccgggttgcaatcaggctctgatgcgcatgactgagttggacaccatcg

SEQ ID NO: 146 - DNA, 47 bp, Artificial Sequence
  Oligomer
tcaggctgagaacgacctgatccgccattatcggttggccgcgagat

SEQ ID NO: 147 - DNA, 35 bp, Artificial Sequence
  Oligomer
gatgacatccatgggtgtgccatttctcacaatcc

SEQ ID NO: 148 - protein, 27 aa, Artificial Sequence
  Oligomer
CCGGGTTGCAATCAGGCTCTGATGCGC

SEQ ID NO: 149 - DNA, 21 bp, Artificial Sequence
  Oligomer
tcaggctgagaacgacctgat

SEQ ID NO: 150 - DNA, 30 bp, Artificial Sequence
  Oligomer
gatcgtttataagtaggaggcctgccttgc

SEQ ID NO: 151 - DNA, 1182 bp, Artificial Sequence
  Figure 7.  Thiostrepton resistance positive selection marker cassette for Propionibacterium acidipropionici, synthetic construct.
gatgacatccatgggtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggacccatgagtccgcgaaagattggcgttaccgagctcgcgctccgcgacgcgcatcagagcctgattgcaacccggatactgagttggacaccatcgcaaatccgtccgatcccgcggtgcagcggatcatcgatgtcaccaagccgtcgcgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtcgacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataatcaggctgagaacgacctgatccgccactcgcggaactccggacgccgcgtcccctcgggggcgcggcgtcctgcatgtccgggcgcaggggcaaggcaggcctcctacttataaacgatc

SEQ ID NO: 152 - DNA, 2950 bp, Artificial Sequence
  Expression cassette for heterologous bifunctional aldehyde/alcohol dehydrogenase of Clostridium carboxidivorans in Propionibacterium acidipropionici, synthetic construct.
gagttctagagtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggacccatgagtccgcgaaagattggcgttaccgagctcgcgctccgcgacgcgcatcagagcctgattgcaacccggatgaaggtcaccaacgtcgaggagctgatgaagaagatgcaggaggtgcagaacgcccagaagaagttcggctccttcacccaggagcaggtcgacgagatcttccgccaggccgcgctggccgcgaactcggcccgcatcgacctggccaagatggccgtcgaggagaccaagatgggcatcgtcgaggacaaggtgatcaagaaccacttcgtcgccgagtacatctacaacaagtacaagaacgagaagacctgcggcatcctggaggaggacgagggcttcggcatggtcaagatcgccgagccggtcggcgtcatcgccgcggtcatcccgaccaccaaccccacctccaccgccatcttcaaggccctcctggccctcaagacccgcaacggcatcatcttctccccgcacccgcgcgccaagaagtgcaccatcgccgcggccaagctggtgctcgacgccgcggtgaaggccggcgccccgaagggcatcatcggctggatcgacgagccctccatcgagctgtcgcagatcgtcatgaaggaggccgacatcatcctggccaccggcggccccggcatggtgaaggccgcgtactcgtccggcaagcccgccatcggcgtcggccccggcaacacccccgccctgatcgacgagtccgccgacatcaagatggccgtcaactccatcctgctgtccaagaccttcgacaacggcatgatctgcgcctccgagcagtcggtggtcgtcgtcgactcgatctacgaggaggtgaagaaggagttcgcccaccgcggcgcctacatcctgtccaaggacgagaccaccaaggtcggcaagatcctcctggtcaacggcaccctgaacgccggcatcgtcggccagtcggcctacaagatcgccgagatggccggcgtgaaggtcccggaggacgccaaggtgctcatcggcgaggtcaagtcggtggagcactccgaggagccgttctcccacgagaagctctcgcccgtcctggccatgtaccgcgccaagaacttcgacgaggccctgctcaaggccggccgcctcgtcgagctgggcgggatgggccacacctcggtcctgtacgtcaacgccatcaccgagaaggtgaaggtggagaagttccgcgagaccatgaagaccggccgcaccctgatcaacatgccctccgcccagggcgccatcggcgacatctacaacttcaagctcgccccctccctgaccctcggctgcggctcctggggcggcaactccgtgtccgagaacgtgggcccgaagcacctgctgaacatcaagtcggtggccgagcgccgcgagaacatgctgtggttccgcgtgccggagaaggtctacttcaagtacggctccctcggcgtcgccctcaaggagctcgacatcctcgacaagaagaaggtgttcatcgtgaccgacaaggtgctgtaccagctgggctacatcgaccgcgtcaccaagatcctcgaggagctcaagatctcctacaagatcttcaccgacgtcgagcccgaccccaccctggccaccgccaagaagggcgccgaggagctgctgtccttcaaccccgacaccatcatcgccgtgggcgggggctccgccatggacgccgccaagatcatgtgggtgatgtacgagcacccggaggtgcgcttcgaggacctcgccatgcgcttcatggacatccgcaagcgcgtctacaccttcccgaagatgggcgagaaggccatgatgatctcggtggccacctcggccggcaccggctcggaggtcacccccttcgccgtcatcaccgacgagaagaccggcgccaagtaccccctggccgactacgagctgaccccgaacatggccatcatcgacgccgagctcatgatgggcatgccgaagggcctcaccgccgcgtccggcatcgacgccctgacccacgcgatcgaggcctacgtgtcgatcatggcctccgagtacaccaacggcctggccctggaggccatccgcctgatcttcaagtacctcccgatcgcctactcggagggcaccacctccatcaaggcccgcgagaagatggcccacgcctcgaccatcgccggcatggccttcgccaacgccttcctcggcgtctgccactcgatggcccacaagctgggctcgacccaccacgtcccccacggcatcgccaacgccctgctgatcaacgaggtgatcaagttcaacgccgtcgagaacccccgcaagcaggccgccttcccgcagtacaagtacccgaacatcaagaagcgctacgcccgcatcgccgactacctcaacctcggcggctcgaccgacgacgagaaggtccagctcctgatcaacgccatcgacgagctcaaggccaagatcaacatcccggagtccatcaaggaggccggcgtcaccgaggagaagttctacgccaccctcgacaagatgtcggagctcgccttcgacgaccagtgcaccggcgccaacccgcgctacccgctcatctccgagatcaagcagatgtacgtgaacgccttctgatgatcaggctgagaacgacctgatccgccactcgcggaactccggacgccgcgtcccctcgggggcgcggcgtcctgcatgtccgggcgcaggggcaaggcaggcctcctacaagcttgagt

SEQ ID NO: 153 - DNA, 2959 bp, Artificial Sequence
  Figure 9.  Expression cassette for heterologous bifunctional aldehyde/alcohol dehydrogenase of Clostridium acetobutylicum in Propionibacterium acidipropionici, synthetic construct.
gagttctagagtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggacccatgagtccgcgaaagattggcgttaccgagctcgcgctccgcgacgcgcatcagagcctgattgcaacccggatgaaggtcaccaccgtcaaggagctggacgagaagctcaaggtcatcaaggaggcccagaagaagttctcgtgctactcgcaggagatggtggacgagatcttccgcaacgccgcgatggccgcgatcgacgcccgcatcgagctcgccaaggccgcggtcctggagaccggcatgggcctcgtcgaggacaaggtgatcaagaaccacttcgccggcgagtacatctacaacaagtacaaggacgagaagacctgcggcatcatcgagcgcaacgagccgtacggcatcaccaagatcgccgagcccatcggcgtcgtcgccgcgatcatccccgtcaccaacccgacctccaccacgatcttcaagtcgctgatctcgctcaagacccgcaacggcatcttcttctcgccgcacccgcgcgccaagaagtcgaccatcctggccgcgaagaccatcctggacgccgcggtcaagtcgggcgcccccgagaacatcatcggctggatcgacgagccctcgatcgagctgacccagtacctgatgcagaaggccgacatcaccctcgccaccggcgggccctcgctcgtcaagtcggcctactcgtccggcaagcccgccatcggcgtggggccgggcaacacccccgtcatcatcgacgagtccgcccacatcaagatggccgtctcctccatcatcctctccaagacctacgacaacggcgtcatctgcgcctcggagcagtccgtgatcgtcctcaagtcgatctacaacaaggtcaaggacgagttccaggagcgcggcgcctacatcatcaagaagaacgagctggacaaggtgcgcgaggtcatcttcaaggacggctcggtgaaccccaagatcgtcggccagtcggcctacaccatcgccgcgatggccggcatcaaggtcccgaagaccacgcgcatcctcatcggcgaggtcacctccctgggcgaggaggagcccttcgcccacgagaagctctcgcccgtcctggccatgtacgaggccgacaacttcgacgacgccctcaagaaggccgtcaccctgatcaacctcggcgggctgggccacacctccggcatctacgccgacgagatcaaggcccgcgacaagatcgaccgcttctcctcggccatgaagaccgtccgcaccttcgtcaacatccccacctcgcagggcgcctccggcgacctgtacaacttccgcatcccgccctccttcaccctcggctgcggcttctgggggggcaactccgtctcggagaacgtgggcccgaagcacctgctgaacatcaagaccgtggccgagcgccgcgagaacatgctgtggttccgcgtcccccacaaggtctacttcaagttcggctgcctccagttcgccctcaaggacctcaaggacctcaagaagaagcgcgccttcatcgtcaccgactcggacccctacaacctgaactacgtcgactccatcatcaagatcctcgagcacctcgacatcgacttcaaggtcttcaacaaggtgggccgcgaggccgacctcaagaccatcaagaaggccaccgaggagatgtcgtccttcatgcccgacaccatcatcgccctgggcgggaccccggagatgtcctccgccaagctgatgtgggtcctctacgagcaccccgaggtcaagttcgaggacctggccatcaagttcatggacatccgcaagcgcatctacaccttccccaagctgggcaagaaggccatgctcgtggccatcaccacgtccgccggctccggctccgaggtcacccccttcgccctcgtgaccgacaacaacaccggcaacaagtacatgctcgccgactacgagatgacccccaacatggccatcgtggacgccgagctcatgatgaagatgccgaagggcctcaccgcctactcgggcatcgacgccctggtcaactcgatcgaggcctacacctccgtctacgcctccgagtacaccaacggcctcgccctcgaggccatccgcctgatcttcaagtacctcccggaggcctacaagaacggccgcaccaacgagaaggcccgcgagaagatggcccacgcgtccaccatggccggcatggcgtccgccaacgccttcctcggcctctgccactccatggccatcaagctgtcctcggagcacaacatcccctccggcatcgccaacgccctcctcatcgaggaggtcatcaagttcaacgccgtggacaacccggtgaagcaggccccctgcccgcagtacaagtaccccaacaccatcttccgctacgcccgcatcgccgactacatcaagctgggcgggaacaccgacgaggagaaggtcgacctcctcatcaacaagatccacgagctcaagaaggccctcaacatcccgacctccatcaaggacgccggcgtgctggaggagaacttctactcctccctggaccgcatctcggagctcgccctggacgaccagtgcaccggcgccaacccgcgcttcccgctcacctccgagatcaaggagatgtacatcaactgcttcaagaagcagccctgatgatcaggctgagaacgacctgatccgccactcgcggaactccggacgccgcgtcccctcgggggcgcggcgtcctgcatgtccgggcgcaggggcaaggcaggcctcctacaagcttgagt

SEQ ID NO: 154 - DNA, 12282 bp, Artificial Sequence
  Synthetic plasmid DNA
ctagaggatccggcggaacttcacgtcctggcggtggagttggcgggcgcgttccagccgttcctccagcacggtgatccgggcctccagacgctcacgctcaccctgctccaggtgccgggtcaccgtcaccgtccgcaccggccgggcctcggcctgggcggcccggcgttcctcactggcccgcttccggcaatcgtcggaacaccacacccggggccgaccccgcccaccgtgggcctccaccggcgccccgcagtggggacacgcccgcagcgccgacgcatcctcatccaaggccatcaccgggtcggaatccatacccgaaaccatatcgtccggacgatgaactgcgccagacagctaagaatgcacgaggtgtgtctccgattctcaggaaacgctcagcattttccgagacgttcggcgcacgcacacacccccacaagaaccgacccgcccagcatccgccgacacgtcgatccgcacccgcgatgggctggccgaggccgactacgaccgctagtcagcacctgcgctgatctaccgtcgccctgaccgactctcccgtcgggattgtcgccggccgctgccagcatggacctgcggccccgccccctcgccctgcaactcgagggaggcggggccgtccaccccccacaccaccccgacaccgtgatgcgcccatgtcgcctaacgggttgcccgacctccccgacatcaagaaaacctgacaccgtcgccgcaagcgctacactgactactagtagtcaggaggtgcgtgatgaccatcgccacatcggtgaaactctccgaagagaccggccgcaaactcgatgaactagcccgggccaccgggcgatccaagtcctactacctgcgcgaggccatcgaggaccacatcgaccagatggtccacgactacgccatcgcccgactcgccgacgacgtgcgagccggccgggccgccacctacagcgccgacgaagtggaccagatccttggcctggacgattgagtacaccgaccccgccgtcaaagcactgcgcaaactcgaccgagcccaggcccgccgcatcaccgcctacatacgtgagctcaccggcctggacgatccccaccaacgcgggaaaggcctcaccgggcccctggccggactctggcgctaccgcgtcggggactaccggatcatctgcgacctgaacgccgaccgcctggccatcatcgccctgaccatcgagcaccgatcccaggcctaccgctgacacgcaaccccgcaccctcggccaagacgtcacacaccacccgccccaccgagcactgaggatgtcaactcgcccgagccggcctgccggccgtcttacgggttgtcttggcgggcggggtgtctttgccctggcccagcagccccacgatctcccgcagcgtgtcggcggtggcggcgtcccgggccgcctgacgctccgcctccgccctggcctgctcggctgcctgcgcccgatcctccgcggcggcggcctgctccctcgcctcggccagctcgccggtcagggcctcgacccgggcctgcacctgccccaggcgcgcctccgcctcctgctgcacctgctcggcccgggcctccgcctggtcccgggccgcctcggcctcggcccggtgctgatccgccagggccgcctcggccaccgcttcggcctgcccatccaccgcctgctcggcccgagccccgaactcctcgcgggccgcatcactcgcctgacgccacgccgccgcccacaccagacccaacggctccgacagatccggcggggccggcgtctggaccgacgccgagacgtcgcgcaggaaccccgccgcagcgtcggtggagcaccccgcctccgccttcaacgaccgcaccgtcacccgccgacccgcaccgctcaaccgcgcataggccgccgccaaccttgacccattcgactccatgacccaccctcccattctgtaccctgtacctgttcctaggtacgttcctaatgtacctcaccggatgcagaacccgcaacccccctcacactccccctgcacggggcccgccccctgcacccccgctgccgcgcccgctcctgcgtcgcggccttgcccctgcccaacgccgggccggcgggcagcccaccagaggctctgtgagacgtcggcgcccccgtccacctaccctaaagaccaaccggccgtggaaacgtctgtgaggagccttgtaggagttcccaggacaagccagcaaggccgggcctgacggcccggaaaggaagtcgctgcgctcctacgaagaagcccctctggggacccccagaccccggaactatctgatttggtttagcggcgtacttccgtcataccggaatttatggcatgctgtggtcatggcgacgacgacggtcgatgagcagtgggagcaggtgtggctgccccgctggcccctggcctccgacgacctggcagcgggcatctaccggatggcccgcccctcggcgctgggggtccgatacatcgaggtcaacccccaagccatcagcaacctcctcgtggtcgactgcgaccaccccgacgctgccatgcgcgccgtctgggaccgccacgactggctgcccaacgccatcgtcgagaaccccgacaacggccacgcccacgccgtgtgggccctggaagcagccatcccgcgcaccgagtacgcccaccgcaagcccatcgcctacgccgccgccgtcaccgagggcctgcgccgatccgtcgacggagacgcctcctacgccggcctgatcaccaagaaccccgaacaccccgcctggaacaccacctggtgcaccgaccacctctaccggctggccgagctcgacacccacctggatgccgccggcctcatgcccgccccctcctggcgacgcacccgccggcgcaaccccgtcggcctgggccgcaactgcgccatcttcgagaccgcccgcacctgggcctaccgcgacgcccgccgcatccgacaacgccacgaatacccgaccgccgaggactcggccgacctgcacgccgtcatcgcctccaccgtcgaggcgctcaacgccggctacagcgaacccctgccggcccgcgaggccgccggcatcgccgccagcatccaccgatggatcacccaccgtttctacggctggatcgactcccacaccgtcaacgaggccactttctccaccatccagagctacagaggacacaagggagccggcaaggctcgtcctcgtgcccgccgtgctgcttctatcaccgattgggaggcatgatggctgacgtccagcaccgcgtgaagcgtcggggcacggcccgcgaggccgcagaacgtgtaggggcctccatccgaaccgcccagcggtggacctccatcccccgtgaggaatggatcactcagaaggccgtcgagcgtgaggagatccgggcctacaagtacgacgaggggcacacgtggggcgagacctcgcgccacttcgggatcgcgaagaccaccgcccaggagcgggcccggcgggctcgaagggagcgggcggccgaagcggagaaggctgccgaggaggccgaggccgcgctgcgtccgacactcttcgagggccaggagcaaggttctgcatgagcaaccccgagtcctcgggtagaccgtctggcccgacgttaagcatggctgaagcggcccgtgcctgtggggtttcagtgtccacggtgaggcgtcaccgtgatgccctggtggcccacggtgctacccgtcatgacgcgtcatgggtgatacccctatcagcgttgatttcatgcggtttgatgccccgggtgacaccccctgatgccccgtcacccaataacgtggcgcctgccatgacgtcccacggtgacgcccccctgacgggggaagtccaagagctgcgcgagcgactggccaacgctgagcatcgagccgagctagccgaagccatcgcggccgagcgacaacacacgatcgacgcccagcgcatcgccttacgggccttagaacccggctcgacccataacagcccggcaaccgatgagccggctaccgctcgcgagcaacctcccggtccagaacccagcgactccaggccacaccgccggagttggtggcgtcggctgactggtggcgcctgaccggccccggtcgtcttcgaggggaacctctcgcctgcgagaggacacagcagccggctgtgctggtagggcatcccagcacgacacccctctgacgcgagaagttcaaggactacgcgaattgctgactaccgccgagcggcagcacacgatcgagatgctcaacgaaccgcactacgcggccttagaaggccccaaggcacgctcacctaccacgtggatcaccaccgatcggcgccgacagctatggaccccatcgcaagatcaaaacccctgagcagccatcgcaccgagcgcccggcacgccggaagaagctccgacgcccctgctgtccggacacggcctaacgcgtccagaccagaaccagtgctccgatctaaaccgaaggcccttcatgtgagagcatagtcgtgacgtcggcacagtagtcgtgcccggcgggggtaacgctacacaacgcttaaaaagcatcggagcaagctaacacagggggactgatgaacaaaacacacaaaatggcgacgctggtaattgccgcgatcttggccgccggaatgaccgcaccaactgcctatgcagattctcctggaaacaccagaattacagccagcgagcaaagcgtccttacccagatactcggccacaaacctacacaaactgaatataaccgatacgttgagacttacggaagcgtaccgaccgaagcagacatcaacgcatatatagaagcgtctgaatctgagggatcatcaagtcaaacggctgctcacgatgactcgacatcacccggcacgagtaccgaaatctacacgcaggcagcccctgccaggttctcaatgtttttcctgtccggaacttggatcactaggagtggtgtagtatcgctctccttgaagccaaggaagggtggtattggcaacgagggggacgagcgtacctggaagactgtatacgacaaattccataacgctgggcaatggacacgatacaagaacaacggcgtagacgccagcatgaaaaagcagtacatgtgccacttcaagtacgggatggtgaagacgccatgggtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggacccatgagtccgcgaaagattggcgttaccgagctcgcgctccgcgacgcgcatcagagcctgattgcaacccggatgactgagttggacaccatcgcaaatccgtccgatcccgcggtgcagcggatcatcgatgtcaccaagccgtcgcgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtcgacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataatcaggctgagaacgacctgatccgccactcgcggaactccggacgccgcgtcccctcgggggcgcggcgtcctgcatgtccgggcgcaggggcaaggcaggcctcctacttataattgtcccatacgcgtcatactggttagtcgctggagatccagacgtttgggacttctatcgttctttatggtggattccagtggcttttctaggaatagtttcaatagtactgatggctagcagtagaggttggggacgacgtctcggcgactccggagaacaccaagtcagggtctcatgagtgtgcgatagcttgagctgtctaccaatctggatatagctatatcggtcgtttgtgtctgattcgccagtgagccaacggcgggggcgacacgcggtggcgaaaccccctggcagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccactagagtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggacccatgagtccgcgaaagattggcgttaccgagctcgcgctccgcgacgcgcatcagagcctgattgcaacccggatgaaggtcaccaacgtcgaggagctgatgaagaagatgcaggaggtgcagaacgcccagaagaagttcggctccttcacccaggagcaggtcgacgagatcttccgccaggccgcgctggccgcgaactcggcccgcatcgacctggccaagatggccgtcgaggagaccaagatgggcatcgtcgaggacaaggtgatcaagaaccacttcgtcgccgagtacatctacaacaagtacaagaacgagaagacctgcggcatcctggaggaggacgagggcttcggcatggtcaagatcgccgagccggtcggcgtcatcgccgcggtcatcccgaccaccaaccccacctccaccgccatcttcaaggccctcctggccctcaagacccgcaacggcatcatcttctccccgcacccgcgcgccaagaagtgcaccatcgccgcggccaagctggtgctcgacgccgcggtgaaggccggcgccccgaagggcatcatcggctggatcgacgagccctccatcgagctgtcgcagatcgtcatgaaggaggccgacatcatcctggccaccggcggccccggcatggtgaaggccgcgtactcgtccggcaagcccgccatcggcgtcggccccggcaacacccccgccctgatcgacgagtccgccgacatcaagatggccgtcaactccatcctgctgtccaagaccttcgacaacggcatgatctgcgcctccgagcagtcggtggtcgtcgtcgactcgatctacgaggaggtgaagaaggagttcgcccaccgcggcgcctacatcctgtccaaggacgagaccaccaaggtcggcaagatcctcctggtcaacggcaccctgaacgccggcatcgtcggccagtcggcctacaagatcgccgagatggccggcgtgaaggtcccggaggacgccaaggtgctcatcggcgaggtcaagtcggtggagcactccgaggagccgttctcccacgagaagctctcgcccgtcctggccatgtaccgcgccaagaacttcgacgaggccctgctcaaggccggccgcctcgtcgagctgggcgggatgggccacacctcggtcctgtacgtcaacgccatcaccgagaaggtgaaggtggagaagttccgcgagaccatgaagaccggccgcaccctgatcaacatgccctccgcccagggcgccatcggcgacatctacaacttcaagctcgccccctccctgaccctcggctgcggctcctggggcggcaactccgtgtccgagaacgtgggcccgaagcacctgctgaacatcaagtcggtggccgagcgccgcgagaacatgctgtggttccgcgtgccggagaaggtctacttcaagtacggctccctcggcgtcgccctcaaggagctcgacatcctcgacaagaagaaggtgttcatcgtgaccgacaaggtgctgtaccagctgggctacatcgaccgcgtcaccaagatcctcgaggagctcaagatctcctacaagatcttcaccgacgtcgagcccgaccccaccctggccaccgccaagaagggcgccgaggagctgctgtccttcaaccccgacaccatcatcgccgtgggcgggggctccgccatggacgccgccaagatcatgtgggtgatgtacgagcacccggaggtgcgcttcgaggacctcgccatgcgcttcatggacatccgcaagcgcgtctacaccttcccgaagatgggcgagaaggccatgatgatctcggtggccacctcggccggcaccggctcggaggtcacccccttcgccgtcatcaccgacgagaagaccggcgccaagtaccccctggccgactacgagctgaccccgaacatggccatcatcgacgccgagctcatgatgggcatgccgaagggcctcaccgccgcgtccggcatcgacgccctgacccacgcgatcgaggcctacgtgtcgatcatggcctccgagtacaccaacggcctggccctggaggccatccgcctgatcttcaagtacctcccgatcgcctactcggagggcaccacctccatcaaggcccgcgagaagatggcccacgcctcgaccatcgccggcatggccttcgccaacgccttcctcggcgtctgccactcgatggcccacaagctgggctcgacccaccacgtcccccacggcatcgccaacgccctgctgatcaacgaggtgatcaagttcaacgccgtcgagaacccccgcaagcaggccgccttcccgcagtacaagtacccgaacatcaagaagcgctacgcccgcatcgccgactacctcaacctcggcggctcgaccgacgacgagaaggtccagctcctgatcaacgccatcgacgagctcaaggccaagatcaacatcccggagtccatcaaggaggccggcgtcaccgaggagaagttctacgccaccctcgacaagatgtcggagctcgccttcgacgaccagtgcaccggcgccaacccgcgctacccgctcatctccgagatcaagcagatgtacgtgaacgccttctgatgatcaggctgagaacgacctgatccgccactcgcggaactccggacgccgcgtcccctcgggggcgcggcgtcctgcatgtccgggcgcaggggcaaggcaggcctcctaca

SEQ ID NO: 155 - DNA, 12291 bp, Artificial Sequence
  Synthetic plasmid DNA
ctagaggatccggcggaacttcacgtcctggcggtggagttggcgggcgcgttccagccgttcctccagcacggtgatccgggcctccagacgctcacgctcaccctgctccaggtgccgggtcaccgtcaccgtccgcaccggccgggcctcggcctgggcggcccggcgttcctcactggcccgcttccggcaatcgtcggaacaccacacccggggccgaccccgcccaccgtgggcctccaccggcgccccgcagtggggacacgcccgcagcgccgacgcatcctcatccaaggccatcaccgggtcggaatccatacccgaaaccatatcgtccggacgatgaactgcgccagacagctaagaatgcacgaggtgtgtctccgattctcaggaaacgctcagcattttccgagacgttcggcgcacgcacacacccccacaagaaccgacccgcccagcatccgccgacacgtcgatccgcacccgcgatgggctggccgaggccgactacgaccgctagtcagcacctgcgctgatctaccgtcgccctgaccgactctcccgtcgggattgtcgccggccgctgccagcatggacctgcggccccgccccctcgccctgcaactcgagggaggcggggccgtccaccccccacaccaccccgacaccgtgatgcgcccatgtcgcctaacgggttgcccgacctccccgacatcaagaaaacctgacaccgtcgccgcaagcgctacactgactactagtagtcaggaggtgcgtgatgaccatcgccacatcggtgaaactctccgaagagaccggccgcaaactcgatgaactagcccgggccaccgggcgatccaagtcctactacctgcgcgaggccatcgaggaccacatcgaccagatggtccacgactacgccatcgcccgactcgccgacgacgtgcgagccggccgggccgccacctacagcgccgacgaagtggaccagatccttggcctggacgattgagtacaccgaccccgccgtcaaagcactgcgcaaactcgaccgagcccaggcccgccgcatcaccgcctacatacgtgagctcaccggcctggacgatccccaccaacgcgggaaaggcctcaccgggcccctggccggactctggcgctaccgcgtcggggactaccggatcatctgcgacctgaacgccgaccgcctggccatcatcgccctgaccatcgagcaccgatcccaggcctaccgctgacacgcaaccccgcaccctcggccaagacgtcacacaccacccgccccaccgagcactgaggatgtcaactcgcccgagccggcctgccggccgtcttacgggttgtcttggcgggcggggtgtctttgccctggcccagcagccccacgatctcccgcagcgtgtcggcggtggcggcgtcccgggccgcctgacgctccgcctccgccctggcctgctcggctgcctgcgcccgatcctccgcggcggcggcctgctccctcgcctcggccagctcgccggtcagggcctcgacccgggcctgcacctgccccaggcgcgcctccgcctcctgctgcacctgctcggcccgggcctccgcctggtcccgggccgcctcggcctcggcccggtgctgatccgccagggccgcctcggccaccgcttcggcctgcccatccaccgcctgctcggcccgagccccgaactcctcgcgggccgcatcactcgcctgacgccacgccgccgcccacaccagacccaacggctccgacagatccggcggggccggcgtctggaccgacgccgagacgtcgcgcaggaaccccgccgcagcgtcggtggagcaccccgcctccgccttcaacgaccgcaccgtcacccgccgacccgcaccgctcaaccgcgcataggccgccgccaaccttgacccattcgactccatgacccaccctcccattctgtaccctgtacctgttcctaggtacgttcctaatgtacctcaccggatgcagaacccgcaacccccctcacactccccctgcacggggcccgccccctgcacccccgctgccgcgcccgctcctgcgtcgcggccttgcccctgcccaacgccgggccggcgggcagcccaccagaggctctgtgagacgtcggcgcccccgtccacctaccctaaagaccaaccggccgtggaaacgtctgtgaggagccttgtaggagttcccaggacaagccagcaaggccgggcctgacggcccggaaaggaagtcgctgcgctcctacgaagaagcccctctggggacccccagaccccggaactatctgatttggtttagcggcgtacttccgtcataccggaatttatggcatgctgtggtcatggcgacgacgacggtcgatgagcagtgggagcaggtgtggctgccccgctggcccctggcctccgacgacctggcagcgggcatctaccggatggcccgcccctcggcgctgggggtccgatacatcgaggtcaacccccaagccatcagcaacctcctcgtggtcgactgcgaccaccccgacgctgccatgcgcgccgtctgggaccgccacgactggctgcccaacgccatcgtcgagaaccccgacaacggccacgcccacgccgtgtgggccctggaagcagccatcccgcgcaccgagtacgcccaccgcaagcccatcgcctacgccgccgccgtcaccgagggcctgcgccgatccgtcgacggagacgcctcctacgccggcctgatcaccaagaaccccgaacaccccgcctggaacaccacctggtgcaccgaccacctctaccggctggccgagctcgacacccacctggatgccgccggcctcatgcccgccccctcctggcgacgcacccgccggcgcaaccccgtcggcctgggccgcaactgcgccatcttcgagaccgcccgcacctgggcctaccgcgacgcccgccgcatccgacaacgccacgaatacccgaccgccgaggactcggccgacctgcacgccgtcatcgcctccaccgtcgaggcgctcaacgccggctacagcgaacccctgccggcccgcgaggccgccggcatcgccgccagcatccaccgatggatcacccaccgtttctacggctggatcgactcccacaccgtcaacgaggccactttctccaccatccagagctacagaggacacaagggagccggcaaggctcgtcctcgtgcccgccgtgctgcttctatcaccgattgggaggcatgatggctgacgtccagcaccgcgtgaagcgtcggggcacggcccgcgaggccgcagaacgtgtaggggcctccatccgaaccgcccagcggtggacctccatcccccgtgaggaatggatcactcagaaggccgtcgagcgtgaggagatccgggcctacaagtacgacgaggggcacacgtggggcgagacctcgcgccacttcgggatcgcgaagaccaccgcccaggagcgggcccggcgggctcgaagggagcgggcggccgaagcggagaaggctgccgaggaggccgaggccgcgctgcgtccgacactcttcgagggccaggagcaaggttctgcatgagcaaccccgagtcctcgggtagaccgtctggcccgacgttaagcatggctgaagcggcccgtgcctgtggggtttcagtgtccacggtgaggcgtcaccgtgatgccctggtggcccacggtgctacccgtcatgacgcgtcatgggtgatacccctatcagcgttgatttcatgcggtttgatgccccgggtgacaccccctgatgccccgtcacccaataacgtggcgcctgccatgacgtcccacggtgacgcccccctgacgggggaagtccaagagctgcgcgagcgactggccaacgctgagcatcgagccgagctagccgaagccatcgcggccgagcgacaacacacgatcgacgcccagcgcatcgccttacgggccttagaacccggctcgacccataacagcccggcaaccgatgagccggctaccgctcgcgagcaacctcccggtccagaacccagcgactccaggccacaccgccggagttggtggcgtcggctgactggtggcgcctgaccggccccggtcgtcttcgaggggaacctctcgcctgcgagaggacacagcagccggctgtgctggtagggcatcccagcacgacacccctctgacgcgagaagttcaaggactacgcgaattgctgactaccgccgagcggcagcacacgatcgagatgctcaacgaaccgcactacgcggccttagaaggccccaaggcacgctcacctaccacgtggatcaccaccgatcggcgccgacagctatggaccccatcgcaagatcaaaacccctgagcagccatcgcaccgagcgcccggcacgccggaagaagctccgacgcccctgctgtccggacacggcctaacgcgtccagaccagaaccagtgctccgatctaaaccgaaggcccttcatgtgagagcatagtcgtgacgtcggcacagtagtcgtgcccggcgggggtaacgctacacaacgcttaaaaagcatcggagcaagctaacacagggggactgatgaacaaaacacacaaaatggcgacgctggtaattgccgcgatcttggccgccggaatgaccgcaccaactgcctatgcagattctcctggaaacaccagaattacagccagcgagcaaagcgtccttacccagatactcggccacaaacctacacaaactgaatataaccgatacgttgagacttacggaagcgtaccgaccgaagcagacatcaacgcatatatagaagcgtctgaatctgagggatcatcaagtcaaacggctgctcacgatgactcgacatcacccggcacgagtaccgaaatctacacgcaggcagcccctgccaggttctcaatgtttttcctgtccggaacttggatcactaggagtggtgtagtatcgctctccttgaagccaaggaagggtggtattggcaacgagggggacgagcgtacctggaagactgtatacgacaaattccataacgctgggcaatggacacgatacaagaacaacggcgtagacgccagcatgaaaaagcagtacatgtgccacttcaagtacgggatggtgaagacgccatgggtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggacccatgagtccgcgaaagattggcgttaccgagctcgcgctccgcgacgcgcatcagagcctgattgcaacccggatgactgagttggacaccatcgcaaatccgtccgatcccgcggtgcagcggatcatcgatgtcaccaagccgtcgcgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtcgacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataatcaggctgagaacgacctgatccgccactcgcggaactccggacgccgcgtcccctcgggggcgcggcgtcctgcatgtccgggcgcaggggcaaggcaggcctcctacttataattgtcccatacgcgtcatactggttagtcgctggagatccagacgtttgggacttctatcgttctttatggtggattccagtggcttttctaggaatagtttcaatagtactgatggctagcagtagaggttggggacgacgtctcggcgactccggagaacaccaagtcagggtctcatgagtgtgcgatagcttgagctgtctaccaatctggatatagctatatcggtcgtttgtgtctgattcgccagtgagccaacggcgggggcgacacgcggtggcgaaaccccctggcagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccactagagtgtgccatttctcacaatcccggggtgcgattgtcgcgtttcccacaggaatcggcgcggggatctggagggtgctgcgacacgcccatattttgaacgatgttcagtgcgtcaacctcgaccccagtgctgaacttgtccgtcgcgggtgcaaggattggacccatgagtccgcgaaagattggcgttaccgagctcgcgctccgcgacgcgcatcagagcctgattgcaacccggatgaaggtcaccaccgtcaaggagctggacgagaagctcaaggtcatcaaggaggcccagaagaagttctcgtgctactcgcaggagatggtggacgagatcttccgcaacgccgcgatggccgcgatcgacgcccgcatcgagctcgccaaggccgcggtcctggagaccggcatgggcctcgtcgaggacaaggtgatcaagaaccacttcgccggcgagtacatctacaacaagtacaaggacgagaagacctgcggcatcatcgagcgcaacgagccgtacggcatcaccaagatcgccgagcccatcggcgtcgtcgccgcgatcatccccgtcaccaacccgacctccaccacgatcttcaagtcgctgatctcgctcaagacccgcaacggcatcttcttctcgccgcacccgcgcgccaagaagtcgaccatcctggccgcgaagaccatcctggacgccgcggtcaagtcgggcgcccccgagaacatcatcggctggatcgacgagccctcgatcgagctgacccagtacctgatgcagaaggccgacatcaccctcgccaccggcgggccctcgctcgtcaagtcggcctactcgtccggcaagcccgccatcggcgtggggccgggcaacacccccgtcatcatcgacgagtccgcccacatcaagatggccgtctcctccatcatcctctccaagacctacgacaacggcgtcatctgcgcctcggagcagtccgtgatcgtcctcaagtcgatctacaacaaggtcaaggacgagttccaggagcgcggcgcctacatcatcaagaagaacgagctggacaaggtgcgcgaggtcatcttcaaggacggctcggtgaaccccaagatcgtcggccagtcggcctacaccatcgccgcgatggccggcatcaaggtcccgaagaccacgcgcatcctcatcggcgaggtcacctccctgggcgaggaggagcccttcgcccacgagaagctctcgcccgtcctggccatgtacgaggccgacaacttcgacgacgccctcaagaaggccgtcaccctgatcaacctcggcgggctgggccacacctccggcatctacgccgacgagatcaaggcccgcgacaagatcgaccgcttctcctcggccatgaagaccgtccgcaccttcgtcaacatccccacctcgcagggcgcctccggcgacctgtacaacttccgcatcccgccctccttcaccctcggctgcggcttctgggggggcaactccgtctcggagaacgtgggcccgaagcacctgctgaacatcaagaccgtggccgagcgccgcgagaacatgctgtggttccgcgtcccccacaaggtctacttcaagttcggctgcctccagttcgccctcaaggacctcaaggacctcaagaagaagcgcgccttcatcgtcaccgactcggacccctacaacctgaactacgtcgactccatcatcaagatcctcgagcacctcgacatcgacttcaaggtcttcaacaaggtgggccgcgaggccgacctcaagaccatcaagaaggccaccgaggagatgtcgtccttcatgcccgacaccatcatcgccctgggcgggaccccggagatgtcctccgccaagctgatgtgggtcctctacgagcaccccgaggtcaagttcgaggacctggccatcaagttcatggacatccgcaagcgcatctacaccttccccaagctgggcaagaaggccatgctcgtggccatcaccacgtccgccggctccggctccgaggtcacccccttcgccctcgtgaccgacaacaacaccggcaacaagtacatgctcgccgactacgagatgacccccaacatggccatcgtggacgccgagctcatgatgaagatgccgaagggcctcaccgcctactcgggcatcgacgccctggtcaactcgatcgaggcctacacctccgtctacgcctccgagtacaccaacggcctcgccctcgaggccatccgcctgatcttcaagtacctcccggaggcctacaagaacggccgcaccaacgagaaggcccgcgagaagatggcccacgcgtccaccatggccggcatggcgtccgccaacgccttcctcggcctctgccactccatggccatcaagctgtcctcggagcacaacatcccctccggcatcgccaacgccctcctcatcgaggaggtcatcaagttcaacgccgtggacaacccggtgaagcaggccccctgcccgcagtacaagtaccccaacaccatcttccgctacgcccgcatcgccgactacatcaagctgggcgggaacaccgacgaggagaaggtcgacctcctcatcaacaagatccacgagctcaagaaggccctcaacatcccgacctccatcaaggacgccggcgtgctggaggagaacttctactcctccctggaccgcatctcggagctcgccctggacgaccagtgcaccggcgccaacccgcgcttcccgctcacctccgagatcaaggagatgtacatcaactgcttcaagaagcagccctgatgatcaggctgagaacgacctgatccgccactcgcggaactccggacgccgcgtcccctcgggggcgcggcgtcctgcatgtccgggcgcaggggcaaggcaggcctcctaca